SEQUENCE LISTING (50 sequences)

SEQ ID NO: 1 - DNA, 83 bp, synthetic construct
  source 1..83: mol_type = other DNA
  modified_base 1: mod_base = OTHER; note = 5' phosphorylated nucleotide
ctgtctcttatacacatctatctctctcttttcctcctcctccgttgttgttgttgagagagatagatgtgtataagagacag

SEQ ID NO: 2 - DNA, 19 bp, synthetic construct
  source 1..19: mol_type = other DNA
agatgtgtataagagacag

SEQ ID NO: 3 - DNA, 99 bp, synthetic construct
  source 1..99: mol_type = other DNA
  modified_base 1: mod_base = OTHER; note = 5' phosphorylated nucleotide
ctgtctcttatacacatctttcttccgatctctctcttttcctcctcctccgttgttgttgttgagagagatcggaagaaagatgtgtataagagacag

SEQ ID NO: 4 - DNA, 27 bp, synthetic construct
  source 1..27: mol_type = other DNA
cggaagaaagatgtgtataagagacag

SEQ ID NO: 5 - DNA, 99 bp, synthetic construct
  source 1..99: mol_type = other DNA
  modified_base 1: mod_base = OTHER; note = 5' phosphorylated nucleotide
ctgtctcttatacacatctttccacacatctctctcttttcctcctcctccgttgttgttgttgagagagatgtgtggaaagatgtgtataagagacag

SEQ ID NO: 6 - DNA, 27 bp, synthetic construct
  source 1..27: mol_type = other DNA
gtgtggaaagatgtgtataagagacag

SEQ ID NO: 7 - DNA, 99 bp, synthetic construct
  source 1..99: mol_type = other DNA
  modified_base 1: mod_base = OTHER; note = 5' phosphorylated nucleotide
ctgtctcttatacacatctttgtcgcaatctctctcttttcctcctcctccgttgttgttgttgagagagattgcgacaaagatgtgtataagagacag

SEQ ID NO: 8 - DNA, 27 bp, synthetic construct
  source 1..27: mol_type = other DNA
tgcgacaaagatgtgtataagagacag

SEQ ID NO: 9 - DNA, 99 bp, synthetic construct
  source 1..99: mol_type = other DNA
  modified_base 1: mod_base = OTHER; note = 5' phosphorylated nucleotide
ctgtctcttatacacatctttagctgcatctctctcttttcctcctcctccgttgttgttgttgagagagatgcagctaaagatgtgtataagagacag

SEQ ID NO: 10 - DNA, 27 bp, synthetic construct
  source 1..27: mol_type = other DNA
gcagctaaagatgtgtataagagacag

SEQ ID NO: 11 - DNA, 99 bp, synthetic construct
  source 1..99: mol_type = other DNA
  modified_base 1: mod_base = OTHER; note = 5' phosphorylated nucleotide
ctgtctcttatacacatcttcctaaggatctctctcttttcctcctcctccgttgttgttgttgagagagatccttaggaagatgtgtataagagacag

SEQ ID NO: 12 - DNA, 27 bp, synthetic construct
  source 1..27: mol_type = other DNA
ccttaggaagatgtgtataagagacag

SEQ ID NO: 13 - DNA, 99 bp, synthetic construct
  source 1..99: mol_type = other DNA
  modified_base 1: mod_base = OTHER; note = 5' phosphorylated nucleotide
ctgtctcttatacacatcttccgttgtatctctctcttttcctcctcctccgttgttgttgttgagagagatacaacggaagatgtgtataagagacag

SEQ ID NO: 14 - DNA, 27 bp, synthetic construct
  source 1..27: mol_type = other DNA
acaacggaagatgtgtataagagacag

SEQ ID NO: 15 - DNA, 99 bp, synthetic construct
  source 1..99: mol_type = other DNA
  modified_base 1: mod_base = OTHER; note = 5' phosphorylated nucleotide
ctgtctcttatacacatcttcgaatcgatctctctcttttcctcctcctccgttgttgttgttgagagagatcgattcgaagatgtgtataagagacag

SEQ ID NO: 16 - DNA, 27 bp, synthetic construct
  source 1..27: mol_type = other DNA
cgattcgaagatgtgtataagagacag

SEQ ID NO: 17 - DNA, 99 bp, synthetic construct
  source 1..99: mol_type = other DNA
  modified_base 1: mod_base = OTHER; note = 5' phosphorylated nucleotide
ctgtctcttatacacatcttcactgtgatctctctcttttcctcctcctccgttgttgttgttgagagagatcacagtgaagatgtgtataagagacag

SEQ ID NO: 18 - DNA, 27 bp, synthetic construct
  source 1..27: mol_type = other DNA
cacagtgaagatgtgtataagagacag

SEQ ID NO: 19 - DNA, 99 bp, synthetic construct
  source 1..99: mol_type = other DNA
  modified_base 1: mod_base = OTHER; note = 5' phosphorylated nucleotide
ctgtctcttatacacatcttgcaggatatctctctcttttcctcctcctccgttgttgttgttgagagagatatcctgcaagatgtgtataagagacag

SEQ ID NO: 20 - DNA, 27 bp, synthetic construct
  source 1..27: mol_type = other DNA
atcctgcaagatgtgtataagagacag

SEQ ID NO: 21 - DNA, 99 bp, synthetic construct
  source 1..99: mol_type = other DNA
  modified_base 1: mod_base = OTHER; note = 5' phosphorylated nucleotide
ctgtctcttatacacatcttatggcgtatctctctcttttcctcctcctccgttgttgttgttgagagagatacgccataagatgtgtataagagacag

SEQ ID NO: 22 - DNA, 27 bp, synthetic construct
  source 1..27: mol_type = other DNA
acgccataagatgtgtataagagacag

SEQ ID NO: 23 - DNA, 99 bp, synthetic construct
  source 1..99: mol_type = other DNA
  modified_base 1: mod_base = OTHER; note = 5' phosphorylated nucleotide
ctgtctcttatacacatcttaccgactatctctctcttttcctcctcctccgttgttgttgttgagagagatagtcggtaagatgtgtataagagacag

SEQ ID NO: 24 - DNA, 27 bp, synthetic construct
  source 1..27: mol_type = other DNA
agtcggtaagatgtgtataagagacag

SEQ ID NO: 25 - DNA, 99 bp, synthetic construct
  source 1..99: mol_type = other DNA
  modified_base 1: mod_base = OTHER; note = 5' phosphorylated nucleotide
ctgtctcttatacacatcttacaagccatctctctcttttcctcctcctccgttgttgttgttgagagagatggcttgtaagatgtgtataagagacag

SEQ ID NO: 26 - DNA, 27 bp, synthetic construct
  source 1..27: mol_type = other DNA
ggcttgtaagatgtgtataagagacag

SEQ ID NO: 27 - DNA, 99 bp, synthetic construct
  source 1..99: mol_type = other DNA
  modified_base 1: mod_base = OTHER; note = 5' phosphorylated nucleotide
ctgtctcttatacacatctctgaccaaatctctctcttttcctcctcctccgttgttgttgttgagagagatttggtcagagatgtgtataagagacag

SEQ ID NO: 28 - DNA, 27 bp, synthetic construct
  source 1..27: mol_type = other DNA
ttggtcagagatgtgtataagagacag

SEQ ID NO: 29 - DNA, 99 bp, synthetic construct
  source 1..99: mol_type = other DNA
  modified_base 1: mod_base = OTHER; note = 5' phosphorylated nucleotide
ctgtctcttatacacatctcctctctaatctctctcttttcctcctcctccgttgttgttgttgagagagattagagaggagatgtgtataagagacag

SEQ ID NO: 30 - DNA, 27 bp, synthetic construct
  source 1..27: mol_type = other DNA
tagagaggagatgtgtataagagacag

SEQ ID NO: 31 - DNA, 99 bp, synthetic construct
  source 1..99: mol_type = other DNA
  modified_base 1: mod_base = OTHER; note = 5' phosphorylated nucleotide
ctgtctcttatacacatctcctgtaacatctctctcttttcctcctcctccgttgttgttgttgagagagatgttacaggagatgtgtataagagacag

SEQ ID NO: 32 - DNA, 27 bp, synthetic construct
  source 1..27: mol_type = other DNA
gttacaggagatgtgtataagagacag

SEQ ID NO: 33 - DNA, 99 bp, synthetic construct
  source 1..99: mol_type = other DNA
  modified_base 1: mod_base = OTHER; note = 5' phosphorylated nucleotide
ctgtctcttatacacatctccgcataaatctctctcttttcctcctcctccgttgttgttgttgagagagatttatgcggagatgtgtataagagacag

SEQ ID NO: 34 - DNA, 27 bp, synthetic construct
  source 1..27: mol_type = other DNA
ttatgcggagatgtgtataagagacag

SEQ ID NO: 35 - DNA, 99 bp, synthetic construct
  source 1..99: mol_type = other DNA
  modified_base 1: mod_base = OTHER; note = 5' phosphorylated nucleotide
ctgtctcttatacacatctcaagtggaatctctctcttttcctcctcctccgttgttgttgttgagagagattccacttgagatgtgtataagagacag

SEQ ID NO: 36 - DNA, 27 bp, synthetic construct
  source 1..27: mol_type = other DNA
tccacttgagatgtgtataagagacag

SEQ ID NO: 37 - DNA, 99 bp, synthetic construct
  source 1..99: mol_type = other DNA
  modified_base 1: mod_base = OTHER; note = 5' phosphorylated nucleotide
ctgtctcttatacacatctgtgcattcatctctctcttttcctcctcctccgttgttgttgttgagagagatgaatgcacagatgtgtataagagacag

SEQ ID NO: 38 - DNA, 27 bp, synthetic construct
  source 1..27: mol_type = other DNA
gaatgcacagatgtgtataagagacag

SEQ ID NO: 39 - DNA, 99 bp, synthetic construct
  source 1..99: mol_type = other DNA
  modified_base 1: mod_base = OTHER; note = 5' phosphorylated nucleotide
ctgtctcttatacacatctggcttcatatctctctcttttcctcctcctccgttgttgttgttgagagagatatgaagccagatgtgtataagagacag

SEQ ID NO: 40 - DNA, 27 bp, synthetic construct
  source 1..27: mol_type = other DNA
atgaagccagatgtgtataagagacag

SEQ ID NO: 41 - DNA, 99 bp, synthetic construct
  source 1..99: mol_type = other DNA
  modified_base 1: mod_base = OTHER; note = 5' phosphorylated nucleotide
ctgtctcttatacacatctggaactacatctctctcttttcctcctcctccgttgttgttgttgagagagatgtagttccagatgtgtataagagacag

SEQ ID NO: 42 - DNA, 27 bp, synthetic construct
  source 1..27: mol_type = other DNA
gtagttccagatgtgtataagagacag

SEQ ID NO: 43 - DNA, 99 bp, synthetic construct
  source 1..99: mol_type = other DNA
  modified_base 1: mod_base = OTHER; note = 5' phosphorylated nucleotide
ctgtctcttatacacatctgacgttagatctctctcttttcctcctcctccgttgttgttgttgagagagatctaacgtcagatgtgtataagagacag

SEQ ID NO: 44 - DNA, 27 bp, synthetic construct
  source 1..27: mol_type = other DNA
ctaacgtcagatgtgtataagagacag

SEQ ID NO: 45 - DNA, 99 bp, synthetic construct
  source 1..99: mol_type = other DNA
  modified_base 1: mod_base = OTHER; note = 5' phosphorylated nucleotide
ctgtctcttatacacatctgagtgtctatctctctcttttcctcctcctccgttgttgttgttgagagagatagacactcagatgtgtataagagacag

SEQ ID NO: 46 - DNA, 27 bp, synthetic construct
  source 1..27: mol_type = other DNA
agacactcagatgtgtataagagacag

SEQ ID NO: 47 - DNA, 99 bp, synthetic construct
  source 1..99: mol_type = other DNA
  modified_base 1: mod_base = OTHER; note = 5' phosphorylated nucleotide
ctgtctcttatacacatctgaagaaggatctctctcttttcctcctcctccgttgttgttgttgagagagatccttcttcagatgtgtataagagacag

SEQ ID NO: 48 - DNA, 27 bp, synthetic construct
  source 1..27: mol_type = other DNA
ccttcttcagatgtgtataagagacag

SEQ ID NO: 49 - DNA, 99 bp, synthetic construct
  source 1..99: mol_type = other DNA
  modified_base 1: mod_base = OTHER; note = 5' phosphorylated nucleotide
ctgtctcttatacacatctaacacctcatctctctcttttcctcctcctccgttgttgttgttgagagagatgaggtgttagatgtgtataagagacag

SEQ ID NO: 50 - DNA, 27 bp, synthetic construct
  source 1..27: mol_type = other DNA
gaggtgttagatgtgtataagagacag

(19) United States
(12) Patent Application Publication
(10) Pub. No.: US 2024/0336965 A1
(43) Pub. Date: Oct. 10, 2024

(54) SENSITIVE MULTIMODAL PROFILING OF NATIVE DNA BY TRANSPOSASE-MEDIATED SINGLE-MOLECULE SEQUENCING

(71) Applicants: THE J. DAVID GLADSTONE INSTITUTES, A TESTAMENTARY TRUST ESTABLISHED UNDER THE WI, San Francisco, CA (US); The Regents of the University of California, Oakland, CA (US); The Board of Trustees of the Leland Stanford Junior University, Stanford, CA (US)

(72) Inventors: Vijay Ramani, San Francisco, CA (US); Ke Wu, Martinez, CA (US); Hani Goodarzi, San Francisco, CA (US); Arjun Scott Nanda, Palo Alto, CA (US); Sivakanthan Kasinathan, Menlo Park, CA (US)

(21) Appl. No.: 18/601,772

(22) Filed: Mar. 11, 2024

Related U.S. Application Data
(60) Provisional application No. 63/489,335, filed on Mar. 9, 2023.

(51) Int. Cl. C12Q 1/6869; C12N 15/10; C12Q 1/44; C12Q 1/48; C12Q 1/6806

(52) U.S. Cl. CPC C12Q 1/6869; C12N 15/1065; C12Q 1/44; C12Q 1/485; C12Q 1/6806; C12Q 2600/154; G01N 2333/91245; G01N 2333/922

(57) ABSTRACT

Methods are provided that implement tagmentation for single-molecule sequencing use 90-99% less input than current protocols: SMRT-Tag, which allows detection of genetic variation and CpG methylation, and SAMOSA-Tag, which uses exogenous adenine methylation to add a third channel for probing chromatin accessibility. SAMOSA-Tag of 30,000-50,000 nuclei resolved single-fiber chromatin structure, CTCF binding, and DNA methylation in patient-derived prostate cancer xenografts and uncovered metastasis-associated global epigenome disorganization.

### CROSS REFERENCE TO RELATED APPLICATIONS

This Application claims the benefit of U.S. Provisional Application 63/489,335 filed on Mar. 9, 2023. The entire contents of this application are incorporated herein by reference in its entirety.

### SEQUENCE LISTING

The instant application contains a Sequence Listing which has been submitted electronically in XML format and is hereby incorporated by reference in its entirety. Said XML copy, created on May 10, 2024, is named 354406_00301_SL.xml and is 61,157 bytes in size.

### FIELD

The present disclosure relates in general to sequencing methods. In particular, the methods relate to sensitive, scalable, and multimodal single-molecule genomics for diverse basic and clinical applications.

### BACKGROUND

Third-generation, single-molecule sequencing (SMS) technologies deliver accurate, multimodal readouts of genetic sequence and nucleobase modifications on kilobase (kb)-to megabase-length nucleic acid templates¹. SMS has facilitated the characterization of previously intractable structural variants and repetitive regions^{2,3}, assembly of gapless human genomes, and high-resolution functional genomics of DNA⁴⁻⁸ and RNA^{9,10}. The intrinsic multimodality of SMS has been exploited by chromatin profiling methods such as the single-molecule adenine methylated oligonucleosome sequencing assay (SAMOSA)^{4.11}, Fiber-seq⁵, nanopore sequencing of nucleosome occupancy and methylome (NanoNOMe)⁷, and others^{6,8,12}. These approaches establish a paradigm for encoding functional genomic information (e.g., histone/transcription factor—DNA interactions) as separate SMS ‘channels’ concurrently with primary sequence and endogenous epigenetic marks such as CpG methylation.

Over the past decade, improvements in cost, data quality, read length, and computational tools have driven rapid maturation of the Pacific Biosciences (PacBio) and Oxford Nanopore (ONT) SMS platforms. For example, the cost of PacBio sequencing has decreased from $2,000 to $35 per gigabase (Gb), concomitant with increases in yield (100 Mb to 90 Gb per instrument run), read length (from ˜1.5 kb to 15-20 kb), and accuracy (from ˜85% to >99.95%)¹³. A key limitation of PacBio SMS remains the amount of input DNA required for PCR-free library preparation (typically at least 1-5 μg, or 150,000-750,000 human cells) owing to sample losses during mechanical or enzymatic fragmentation, adaptor ligation, and serial reaction cleanups. While low-input protocols are available, they typically rely on PCR amplification, which erases modified bases and may introduce biases. This obstacle has limited the primary use of SMS to genome assembly and medical genetics, precluding analyses of rare clinical samples and post-mitotic cell populations, single cells, and microorganisms.

### SUMMARY

Embodiments are directed to single cell sequencing methods that implement tagmentation use 90-99% less input than current protocol and do not require the step of amplification of DNA.

In one aspect, a method of genome and epigenome sequencing, comprises isolating DNA sequences, obtaining one or more cells or nuclei from a sample; conducting a tagmentation reaction with a hyperactive transposase on the isolated DNA sequences cells or nuclei to produce a plurality of nucleic acid libraries; repairing gaps in nucleic libraries; fractionating the nucleic acid libraries; and, sequencing the nucleic acid libraries. In certain embodiments, the isolated DNA sequence concentration is in a range from about 10 ng to about 100 ng. In certain embodiments, the isolated DNA sequence concentration is in a range from about 20 ng to about 90 ng. In certain embodiments, the isolated DNA sequence concentration is in a range from about 20 ng to about 90 ng. In certain embodiments, the isolated DNA sequence concentration is in a range from about 30 ng to about 80 ng. In certain embodiments, the isolated DNA sequence concentration about 35 ng to about 60 ng. In certain embodiments, the isolated DNA sequence concentration is about 40 ng. In certain embodiments, a plurality of cells or nuclei are subjected to the tagmentation reaction. In certain embodiments, a single cell or nucleus is subjected to the tagmentation reaction. In certain embodiments, the hyperactive transposase controls fragment size based on concentration of the isolated DNA sequences. In certain embodiments, the hyperactive transposase comprises hairpin oligonucleotides to generate long fragments. In certain embodiments, long fragments generated comprise up to about 150,000 base pairs. In certain embodiments, a generated fragment comprises about 100 base pairs to about 150,000. In certain embodiments, the hyperactive transposase is prokaryotic, eukaryotic or proteases. In certain embodiments, the prokaryotic hyperactive transposases comprise Tn5, Tn5 mutants, Tn5 derivatives, Tn7, Tn10, phages or combinations thereof. In certain embodiments, a Tn5 mutant comprises one or more mutations. In certain embodiments, the Tn5 mutant comprises an R27S, an E54K, an L372P substitution or combinations thereof. In certain embodiments, a Tn5 derivative is linked to an epitope comprising protein A, nanobodies, biotin, streptavidin, protein G, FK-binding protein, beads or combinations thereof. In certain embodiments, the protease transposases comprise casposases, Cas9 or combinations thereof. In certain embodiments, the eukaryotic transposases comprise retrotransposons (class I transposons), class II transposons or miniature inverted-repeat transposable elements (MITEs, or class III transposons). In certain embodiments, the eukaryotic transposases comprise Sleeping Beauty transposon system (SBTS), piggyBac (PB) transposons, Hermes transposons or combinations thereof. In certain embodiments, the sequencing is a high-throughput sequencing reaction. In certain embodiments, the sequencing is a single molecule sequencing (SMS) method. In certain embodiments, the ratio of transposase: DNA is from about 1×10⁻⁵ to 1×10⁻³ picomoles of per ng of DNA. In certain embodiments, the ratio of transposase: DNA is from about 5×10⁻⁴ to 10×10⁻³ picomoles of per ng of DNA. In certain embodiments, the tagmentation reaction is conducted at a temperature between 15° C. to about 75° C. In certain embodiments, the tagmentation reaction is conducted at a temperature of about 55° C. In certain embodiments, the libraries comprise one or more multiplexed nucleic acid sequences. In certain embodiments, each transposon further comprises a unique barcode. In certain embodiments, the sample is a biological sample. In certain embodiments, the method does not comprise the step of amplification of the libraries.

In another aspect, a nucleic acid sequencing assay comprises modifying one or more cells or cell nuclei in situ; tagmenting the cells or cell nuclei with a hairpin-loaded hyperactive transposon; extracting DNA from the cells or cell nuclei; conducting gap repair of the extracted DNA; and, sequencing of the DNA. In certain embodiments, the modification comprises methylation, acetylation, phosphorylation, ubiquitination, sumoylation or combinations thereof. In certain embodiments, the modification comprises methylation. In certain embodiments, the cells or cell nuclei are simultaneously subjected to nucleolytic cleavage and DNA modification. In certain embodiments, the cells or cell nuclei are subjected to nucleolytic cleavage after DNA modification. In certain embodiments, the nucleolytic cleavage is conducted by a nuclease. In certain embodiments, the nuclease is a micrococcal nuclease (MNase). In certain embodiments, the one or more cell nuclei comprise from about 500 cells or cell nuclei to about 200,000 cells or cell nuclei. In certain embodiments, the one or more cells or cell nuclei comprise from about 750 cells or cell nuclei to about 150,000 cells or cell nuclei. In certain embodiments, the one or more cells or cell nuclei comprise from about 1000 cells or cell nuclei to about 100,000 cells or cell nuclei. In certain embodiments, the one or more cells or cell nuclei comprises a single cell or nucleus. In certain embodiments, the hyperactive transposase controls fragment size based on concentration of the isolated DNA sequences. In certain embodiments, the hyperactive transposase comprises hairpin oligonucleotides to generate long fragments. In certain embodiments, long fragments generated comprise up to about 150,000 base pairs. In certain embodiments, a generated fragment comprises about 100 base pairs to about 150,000. In certain embodiments, the hyperactive transposase is prokaryotic, eukaryotic or proteases. In certain embodiments, the prokaryotic hyperactive transposases comprise Tn5, Tn5 mutants, TN5 derivatives, Tn7, Tn10, phages or combinations thereof. In certain embodiments, a Tn5 mutant comprises one or more mutations. In certain embodiments, the Tn5 mutant comprises an R27S, an E54K, an L372P substitution or combinations thereof. In certain embodiments, a Tn5 derivative is linked to an epitope comprising protein A, nanobodies, biotin, streptavidin, protein G, FK-binding protein, beads or combinations thereof. In certain embodiments, the protease transposases comprise casposases, Cas9 or combinations thereof. In certain embodiments, the eukaryotic transposases comprise retrotransposons (class I transposons), class II transposons or miniature inverted-repeat transposable elements (MITEs, or class III transposons). In certain embodiments, the eukaryotic transposases comprise Sleeping Beauty transposon system (SBTS), piggyBac (PB) transposons, Hermes transposons or combinations thereof. In certain embodiments, the sequencing is a high-throughput sequencing reaction. In certain embodiments, the sequencing is a single molecule sequencing (SMS) method. In certain embodiments, ratio of transposase: DNA is from about 1×10⁻⁵ to 1×10⁻³ picomoles of per ng of DNA. In certain embodiments, the ratio of transposase: DNA is from about 5×10⁻⁴ to 10×10⁻³ picomoles of per ng of DNA. In certain embodiments, the tagmentation reaction is conducted at a temperature between 15° C. to about 75° C. In certain embodiments, the tagmentation reaction is conducted at a temperature of about 55° C. In certain embodiments, the libraries comprise one or more multiplexed nucleic acid sequences. In certain embodiments, each transposon further comprises a unique barcode. In certain embodiments, the sample is a biological sample. In certain embodiments, the method does not comprise the step of amplification of the libraries.

In another aspect, a nucleic acid sequencing assay comprises modifying one or more cells or cell nuclei ex situ; tagmenting the cells or cell nuclei with a hairpin-loaded hyperactive transposon; extracting DNA from the cells or cell nuclei; conducting gap repair of the extracted DNA; and, sequencing of the DNA. In certain embodiments, the modification comprises methylation, acetylation, phosphorylation, ubiquitination, sumoylation or combinations thereof. In certain embodiments, the modification comprises methylation. In certain embodiments, the cell nuclei are simultaneously subjected to nucleolytic cleavage and DNA modification. In certain embodiments, the cell nuclei are subjected to nucleolytic cleavage after DNA modification. In certain embodiments, the nucleolytic cleavage is conducted by a nuclease. In certain embodiments, the nuclease is a micrococcal nuclease (MNase). In certain embodiments, the one or more cells or cell nuclei comprise from about 500 cells or cell nuclei to about 200,000 cells or cell nuclei. In certain embodiments, the one or more cells or cell nuclei comprise from about 750 cells or cell nuclei to about 150,000 cells or cell nuclei. In certain embodiments, the one or more cells or cell nuclei comprises from about 1000 cells or cell nuclei to about 100,000 cells or cell nuclei. In certain embodiments, the one or more cells or cell nuclei comprise a single nucleus. In certain embodiments, the hyperactive transposase controls fragment size based on concentration of the isolated DNA sequences. In certain embodiments, the hyperactive transposase comprises hairpin oligonucleotides to generate long fragments. In certain embodiments, long fragments generated comprise up to about 150,000 base pairs. In certain embodiments, a generated fragment comprises about 100 base pairs to about 150,000. In certain embodiments, the hyperactive transposase is prokaryotic, eukaryotic or proteases. In certain embodiments, the prokaryotic hyperactive transposases comprise Tn5, Tn5 mutants, Tn5 derivatives, Tn7, Tn10, phages or combinations thereof. In certain embodiments, a Tn5 mutant comprises one or more mutations. In certain embodiments, the Tn5 mutant comprises an R27S, an E54K, an L372P substitution or combinations thereof. In certain embodiments, a Tn5 derivative is linked to an epitope comprising protein A, nanobodies, biotin, streptavidin, protein G, FK-binding protein, beads or combinations thereof. In certain embodiments, the protease transposases comprise casposases, Cas9 or combinations thereof. In certain embodiments, the eukaryotic transposases comprise retrotransposons (class I transposons), class II transposons or miniature inverted-repeat transposable elements (MITEs, or class III transposons). In certain embodiments, the eukaryotic transposases comprise Sleeping Beauty transposon system (SBTS), piggyBac (PB) transposons, Hermes transposons or combinations thereof. In certain embodiments, the sequencing is a high-throughput sequencing reaction. In certain embodiments, the sequencing is a single molecule sequencing (SMS) method In certain embodiments, a ratio of transposase: DNA is from about 1×10⁻⁵ to 1×10⁻³ picomoles of per ng of DNA. In certain embodiments, a ratio of transposase: DNA is from about 5×10⁻⁴ to 10×10⁻³ picomoles of per ng of DNA. In certain embodiments, the tagmentation reaction is conducted at a temperature between 15° C. to about 75° C. In certain embodiments, the tagmentation reaction is conducted at a temperature of about 55° C. In certain embodiments, the libraries comprise one or more multiplexed nucleic acid sequences. In certain embodiments, each transposon further comprises a unique barcode. In certain embodiments, the sample is a biological sample. In certain embodiments, the method does not comprise the step of amplification of the libraries.

In another aspect, a method for identifying DNA sequence, CpG methylation, or single-fiber chromatin accessibility to exogenous adenine methyltransferases comprises obtaining a biological sample and conducting the assays embodied herein.

Each embodiment disclosed herein is contemplated as being applicable to each of the other disclosed embodiments. Thus, all combinations of the various elements described herein are within the scope of the disclosure.

### DEFINITIONS

The terminology used herein is for the purpose of describing particular embodiments only and is not intended to be limiting of the disclosure. Unless specifically defined otherwise, all technical and scientific terms used herein shall be taken to have the same meaning as commonly understood by one of ordinary skill in the art (e.g., sequencing techniques, cell culture, molecular genetics, biochemistry, etc.).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, e.g. within 5-fold, within 2-fold etc., of a value. Where particular values are described in the application and claims, unless otherwise stated the term “about” meaning within an acceptable error range for the particular value should be assumed. All numeric values are herein assumed to be modified by the term “about”, whether or not explicitly indicated.

The recitation of numerical ranges by endpoints includes all numbers within that range (e.g., 1 to 5 includes 1, 1.01, 1.1, 1.5, 2, 2.75, 3, 3.80, 4, and 5). Although some suitable dimensions ranges and/or values pertaining to various components, features and/or specifications are disclosed, one of skill in the art, incited by the present disclosure, would understand desired dimensions, ranges and/or values may deviate from those expressly disclosed.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads. Nucleic acids comprising a barcode sequence that are optionally configured to interact with a nucleic acid to generate a barcoded nucleic acid may be referred to as a nucleic acid barcode molecule.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof-and that other elements can be included and still fall within the scope/definition of the defined item, composition, apparatus, method, process, system, etc.

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

As used in this specification and the appended claims, the term “or” is generally employed in its sense including “and/or” unless the content clearly dictates otherwise.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1E are a series of schematics and plots demonstrating that tagmentation enables tunable single-molecule real time (SMRT) sequencing. (FIG. 1A) In SMRT-Tag, hairpin adaptor-loaded Tn5 transposase is used to fragment DNA into kilobase (kb)-scale fragments. The 9-nt gaps introduced by transposition are closed via optimized gap repair and exonuclease digestion enriches for covalently closed templates required for PacBio sequencing. (FIG. 1B) Varying concentration of hairpin-loaded transposomes and reaction temperature tunes fragmentation of genomic DNA over a size range of 2-10 kb. (FIG. 1C) PacBio Circular consensus sequencing (CCS) fragment lengths for SMRT-Tag libraries fractionated into short and long molecules optimal for PacBio polymerases 2.1 (light purple) and 2.2 (dark purple) chemistries, respectively. The distribution for the long-fragment library (2.2 chemistry) has a tail that extends beyond 20-kb. (FIG. 1D) Empiric quality score (Q-score) distributions for 2.1 and 2.2 libraries. (FIG. 1E) Heatmap of logarithmically scaled counts of CCS length as a function of number of CCS passes per molecule.

FIGS. 2A-2G are a series of plots, graphs and a schematic demonstrating that SMRT-Tag enables accurate genotyping and epigenotyping of low-input samples. (FIG. 2A) To establish whether low-input SMRT-Tag libraries can be sequenced to sufficient depth, 40 ng gDNA (equivalent to ˜7,000 human cells) were tagmented from Genome in a Bottle (GIAB) reference individual HG002 and the resulting library was sequenced on a single flow cell. (FIG. 2B) Read length distribution of the 40 ng SMRT-Tag library. Precision, recall, and F1 scores for (FIG. 2C) Deep Variant single nucleotide variant (SNV) and insertion/deletion (indel) calls and (FIG. 2D) pbsv structural variant (SV) calls from 40 ng SMRT-Tag and coverage-matched ligation-based PacBio data compared against GIAB HG002 variant calling benchmarks. (FIG. 2E) Precision, recall, and number of true positive calls for SVs binned by size for 40 ng SMRT-Tag and coverage-matched ligation-based data benchmarked against GIAB HG002 SV calls. (FIG. 2F) Comparison of SMRT-Tag primrose and HG002 bisulfite CpG methylation. (FIG. 2G) Receiver operating characteristic (ROC) curves for CpG methylation detected using 40 ng SMRT-Tag, pooled SMRT-Tag (not coverage matched), and ligation-based PacBio compared against bisulfite sequencing.

FIGS. 3A-3E are a series of schematics and plots demonstrating SAMOSA-Tag: Single-molecule chromatin profiling via tagmentation of adenine-methylated nuclei. (FIG. 3A) In SAMOSA-Tag, nuclei are methylated using the nonspecific EcoGII m⁶dAase and tagmented in situ with hairpin-loaded transposomes. DNA is purified, gap-repaired, and sequenced, resulting in molecules where ends result from Tn5 transposition, m⁶dA marks represent fiber accessibility, and computationally defined unmethylated ‘footprints’ capture protein-DNA interactions. (FIG. 3B) Length distribution for SAMOSA-Ta molecules from OS152 osteosarcoma cells. (FIG. 3C) Average methylation from the first 1-kb of molecules and (FIG. 3D) unmethylated footprint size distribution for the same data as in FIG. 3B. (FIG. 3E) Genome browser visualization SAMOSA-Tag molecules at the amplified MYC (locus. Predicted accessible and inaccessible bases are marked in purple and blue, respectively. Average SAMOSA accessibility is shown in purple; matched ATAC-seq track shown in blue.

FIGS. 4A-4F are a series of plots and heat maps demonstrating that SAMOSA-

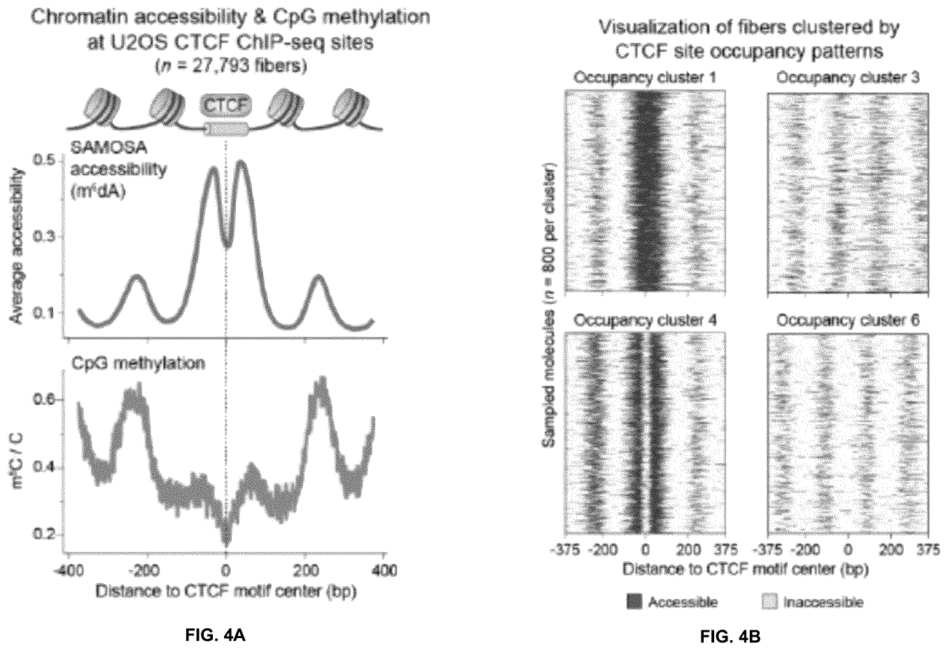

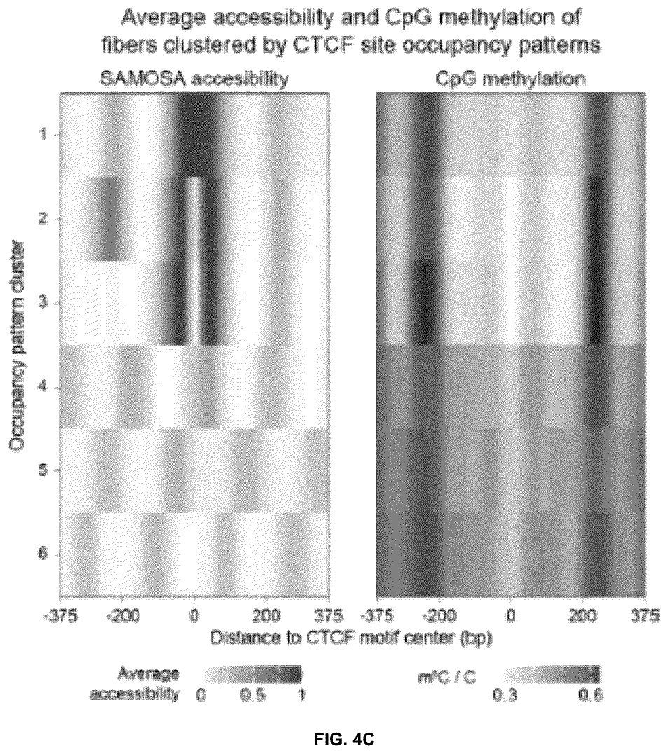

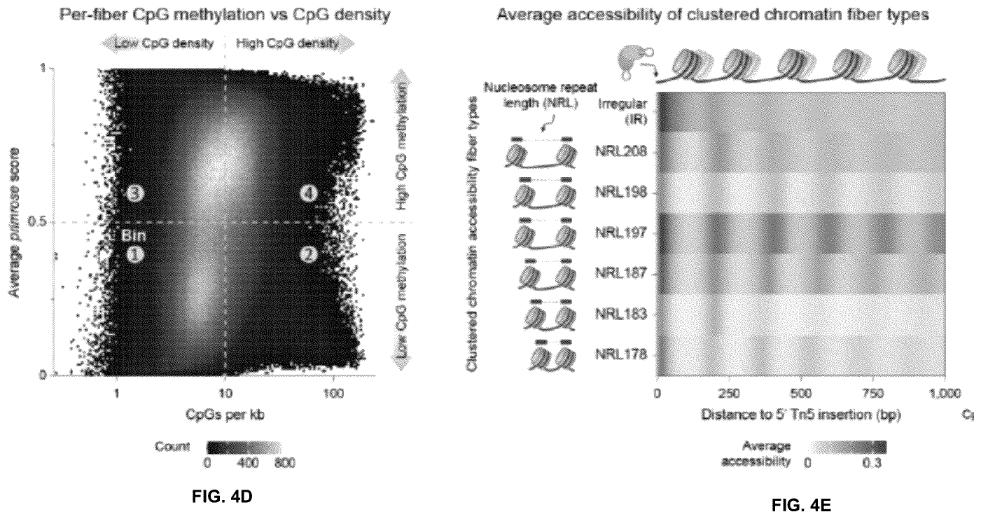

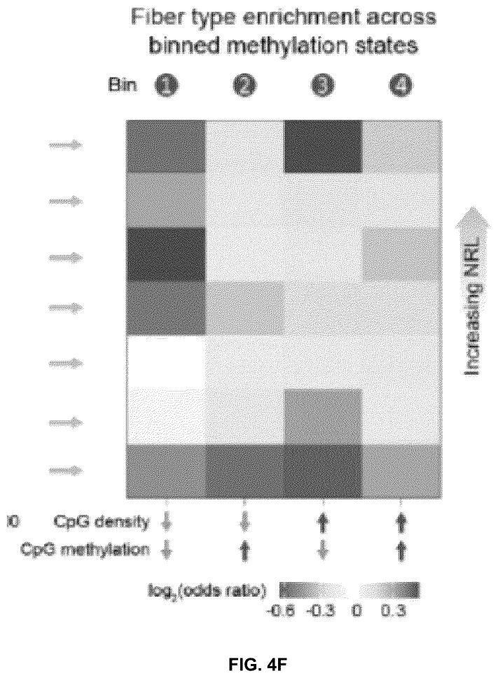

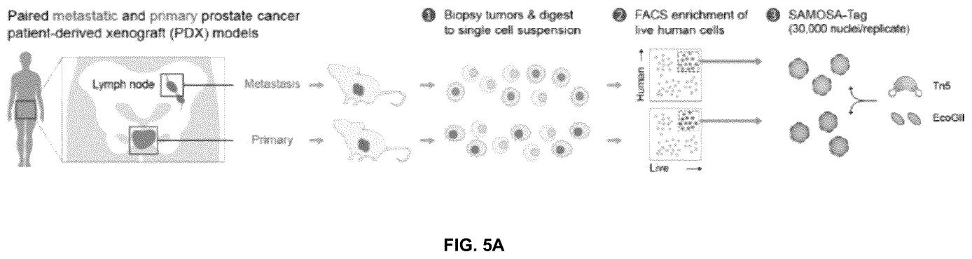

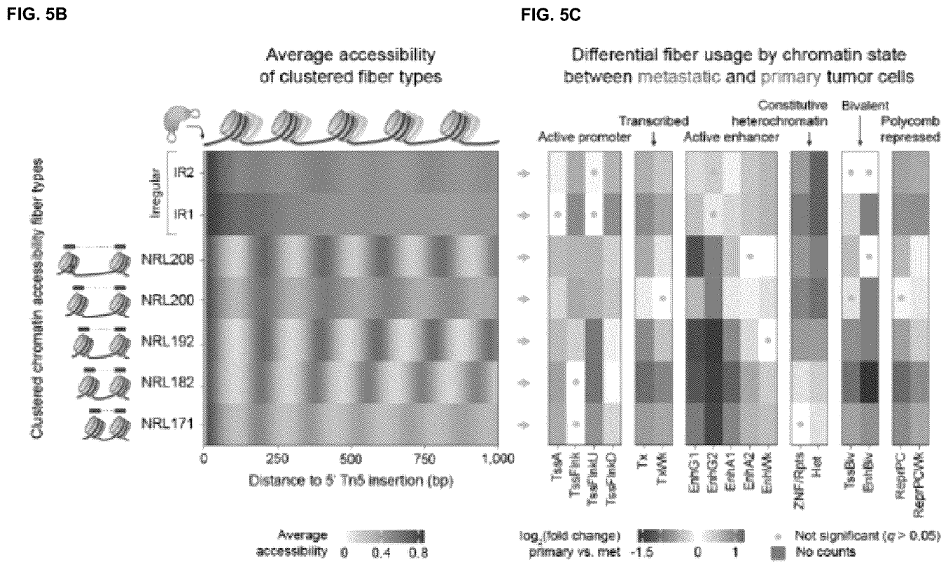

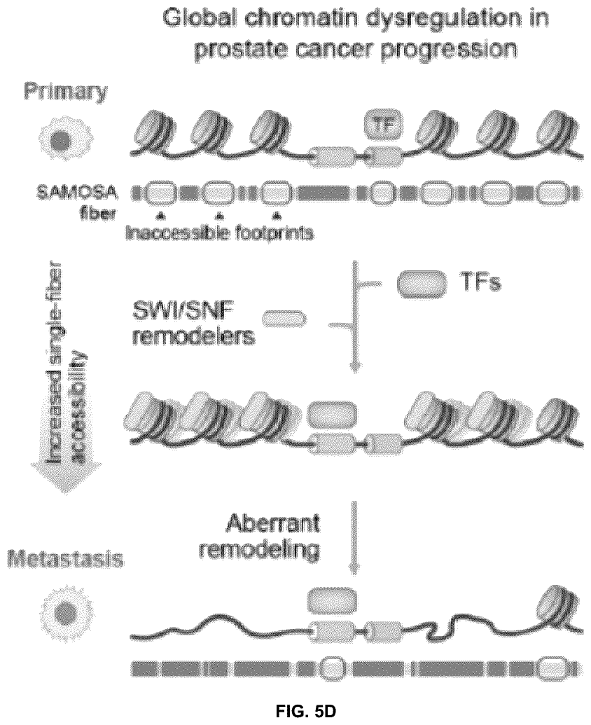

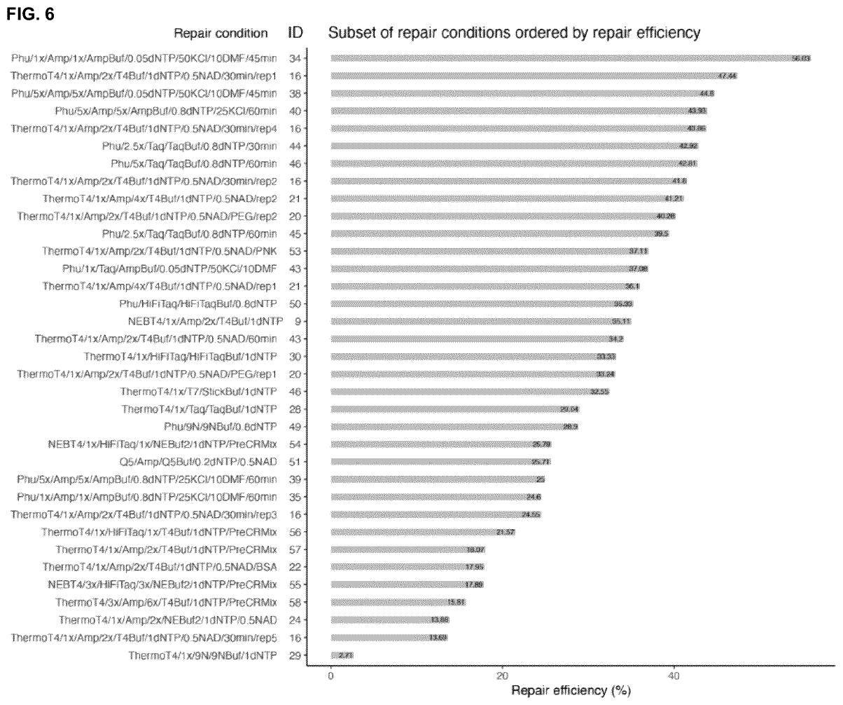

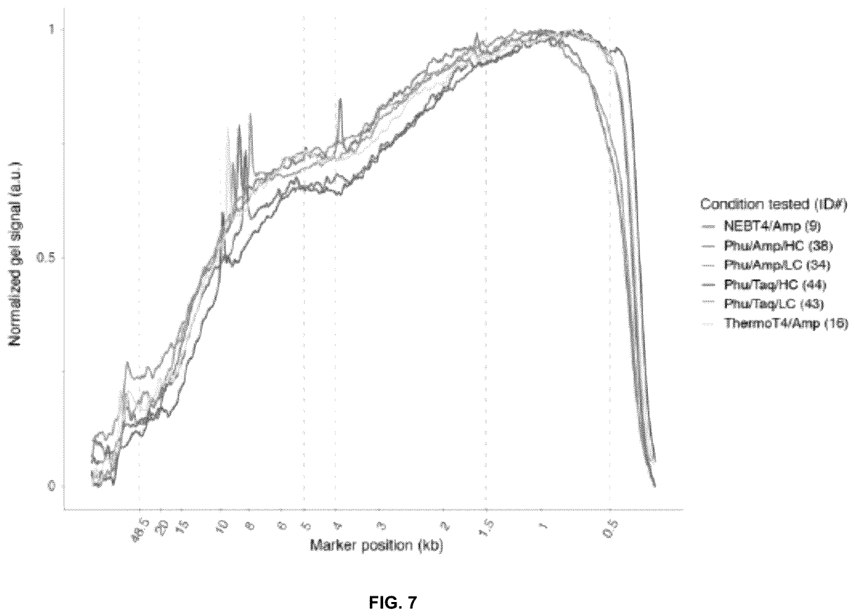

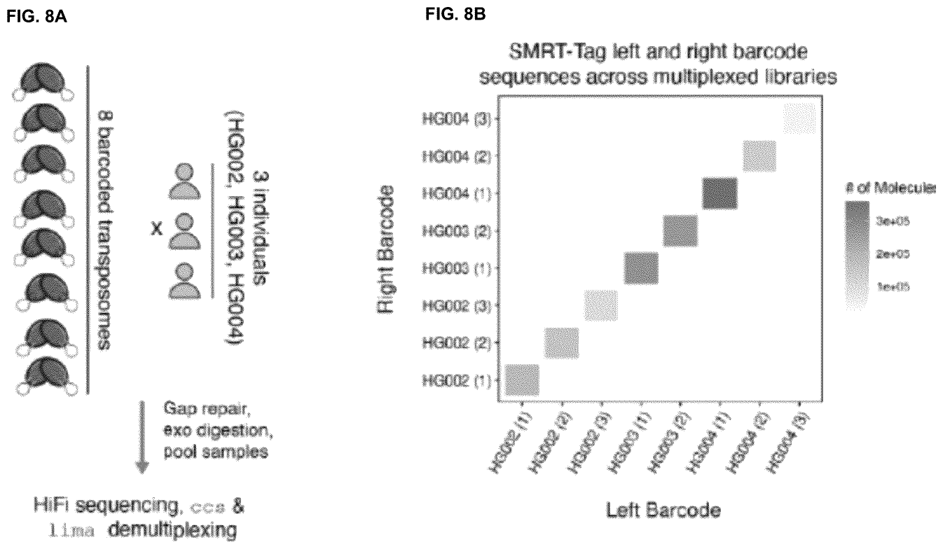

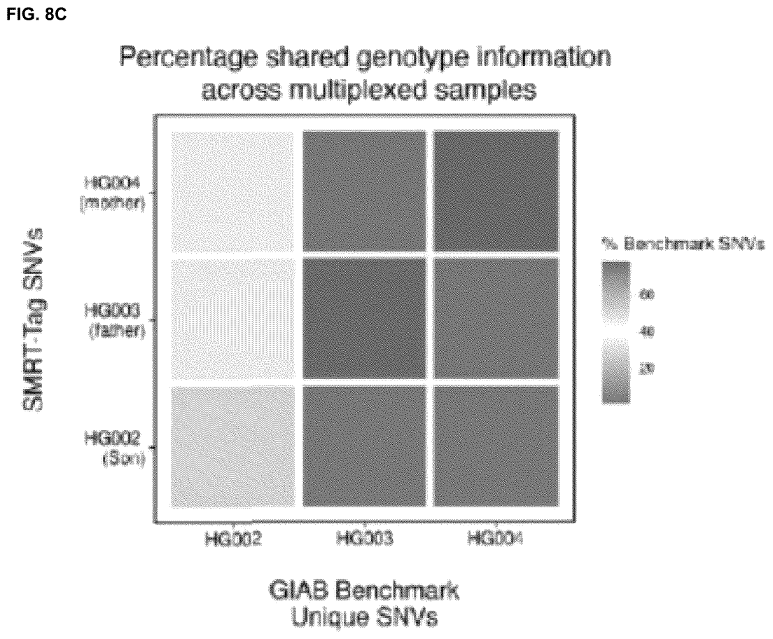

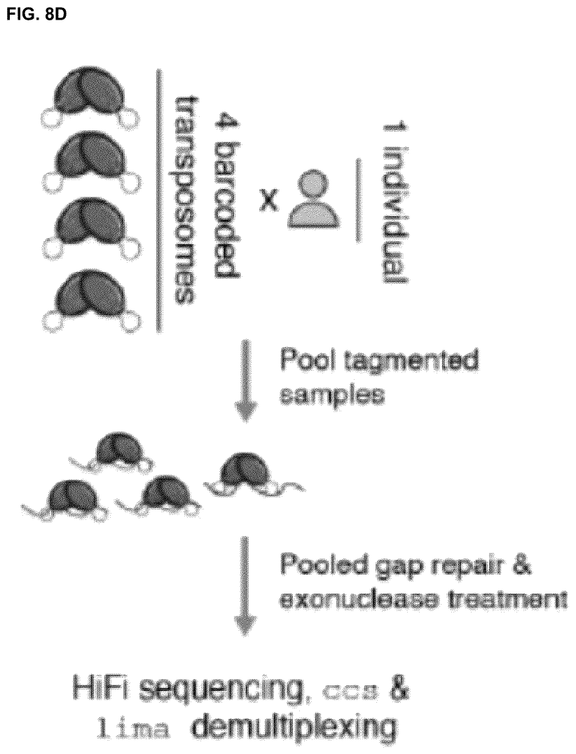

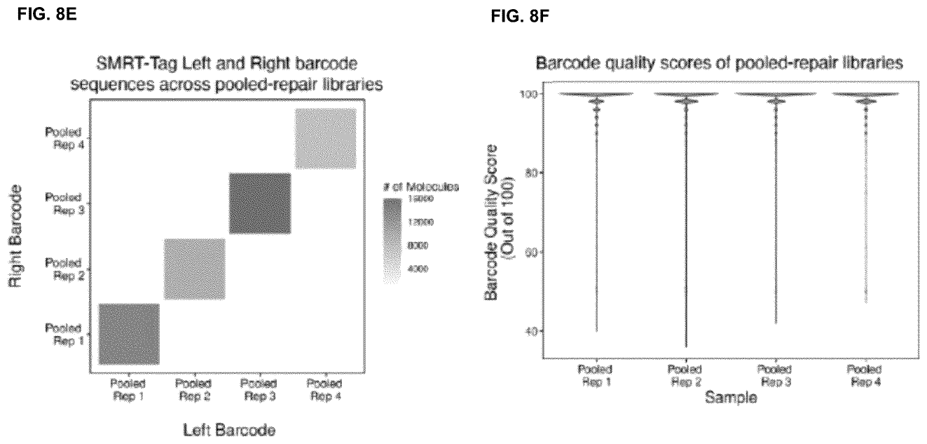

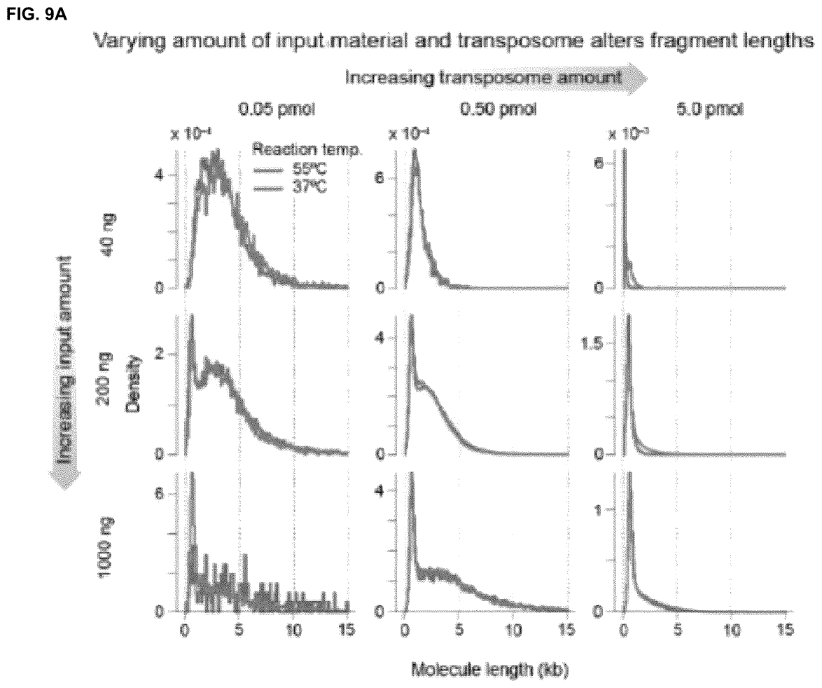

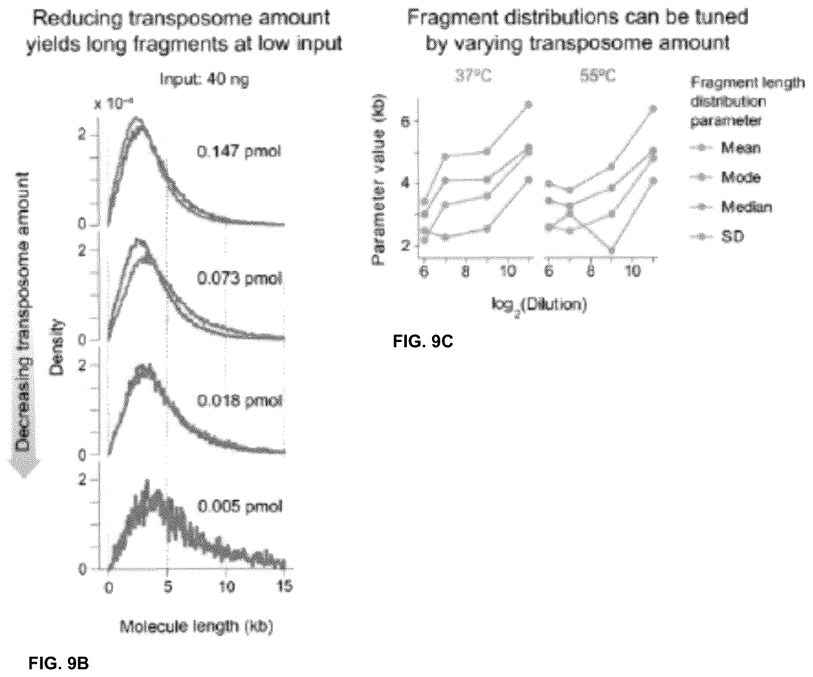

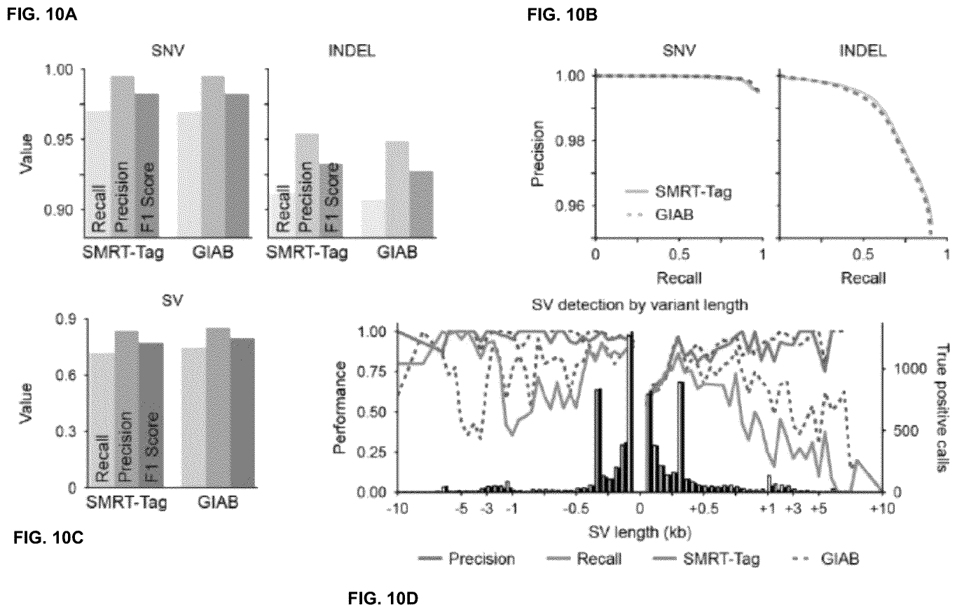

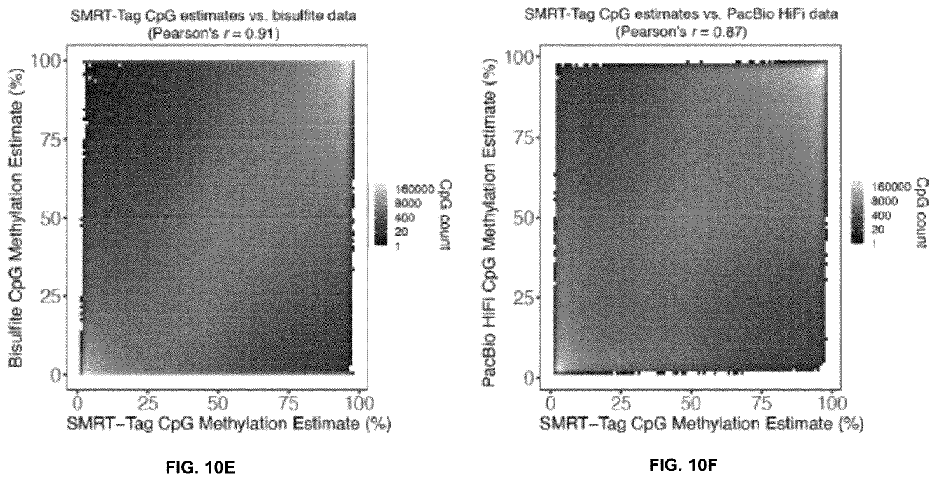

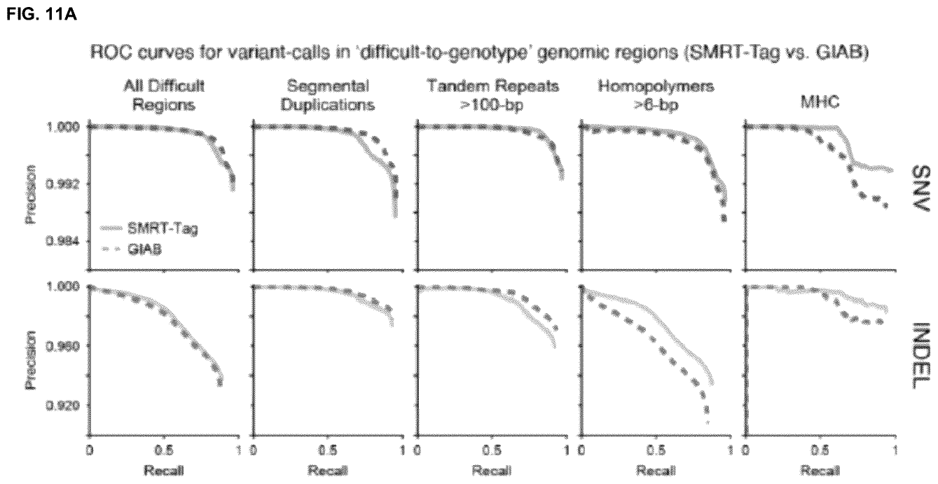

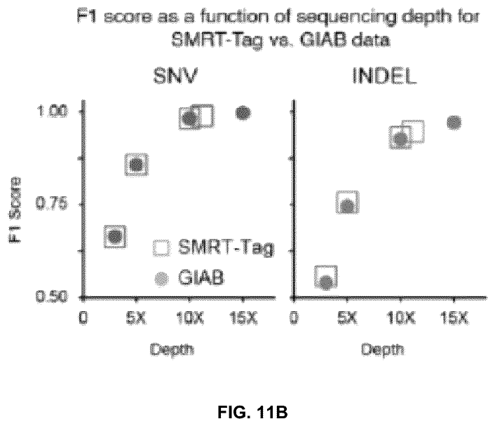

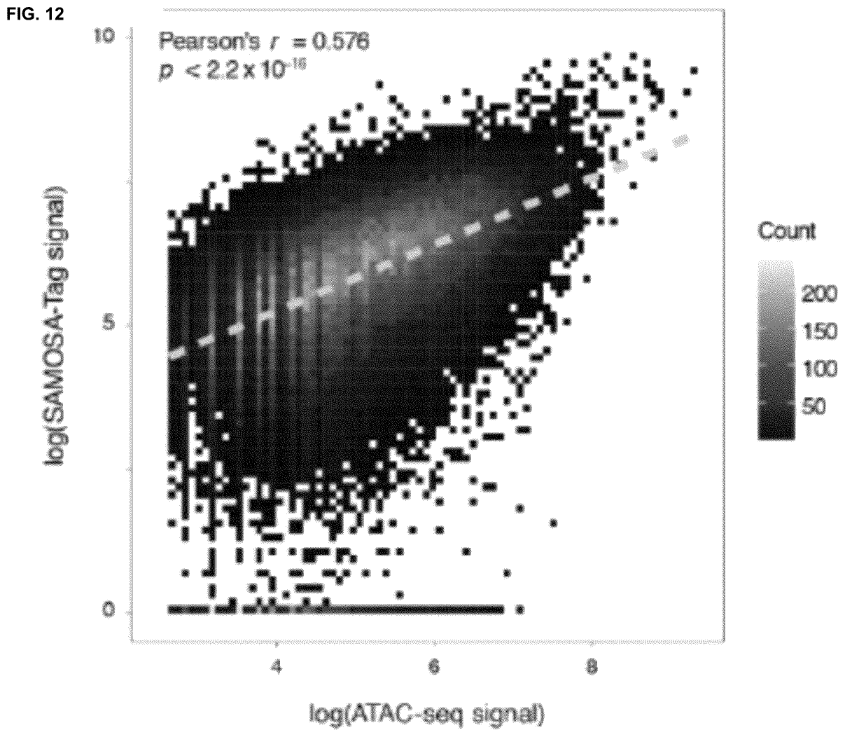

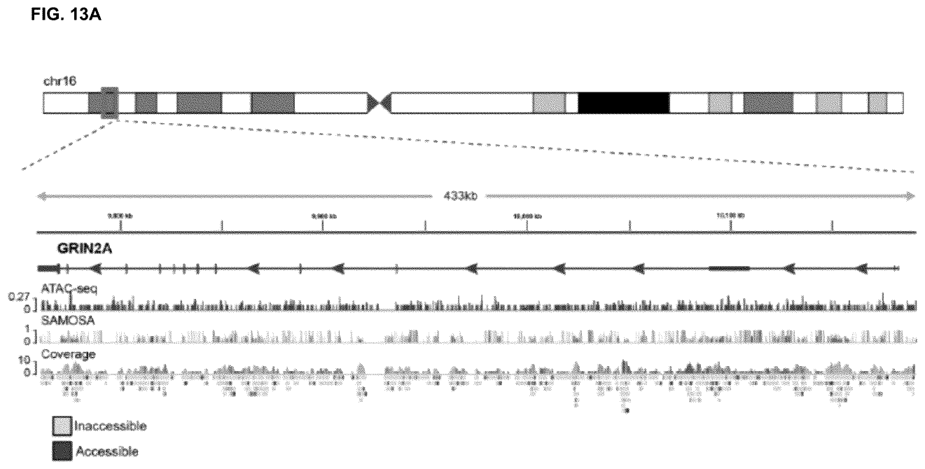

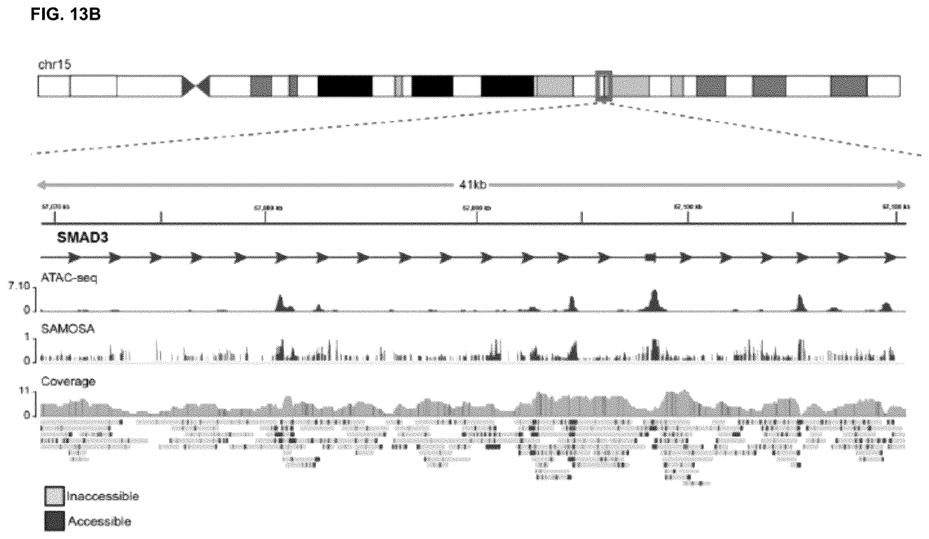

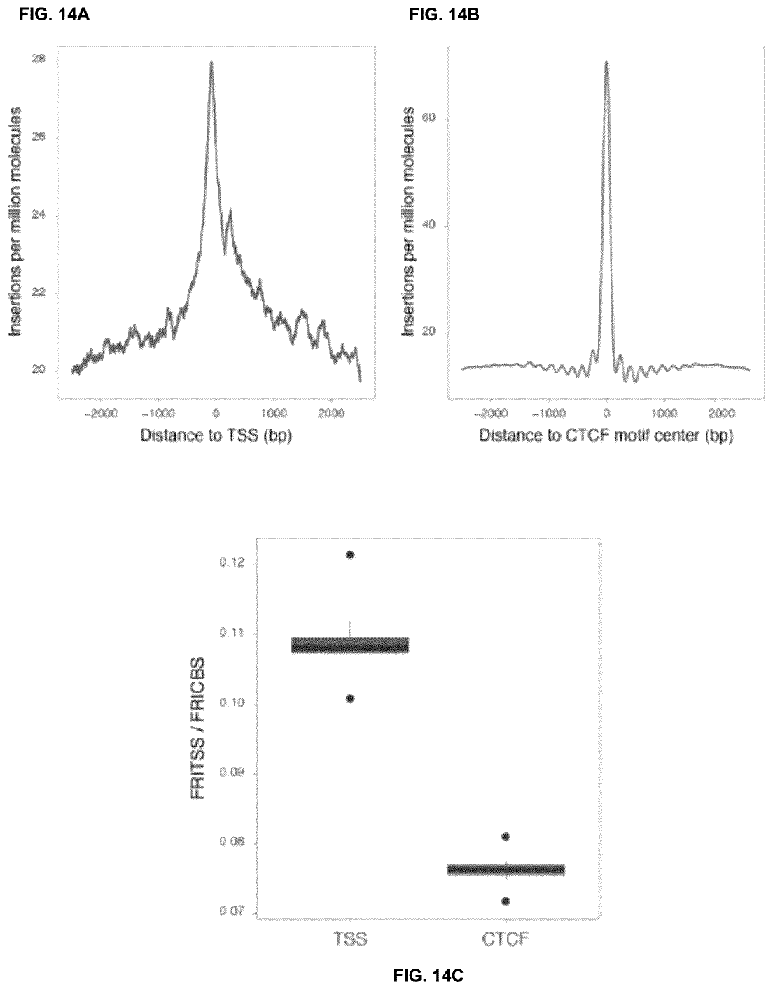

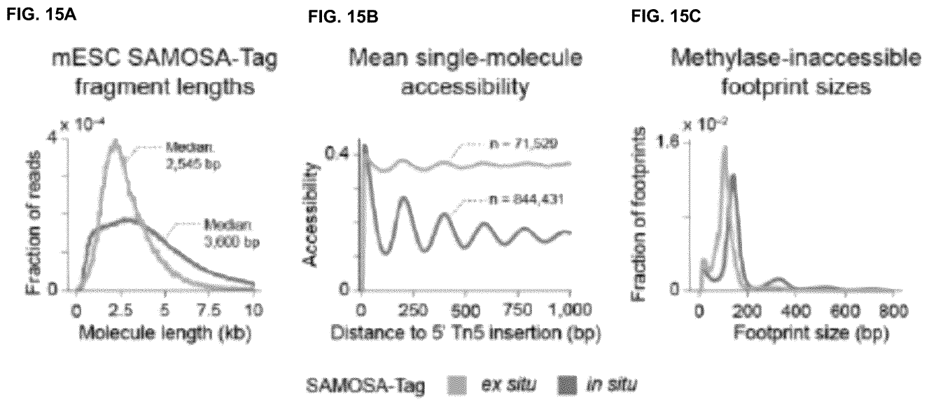

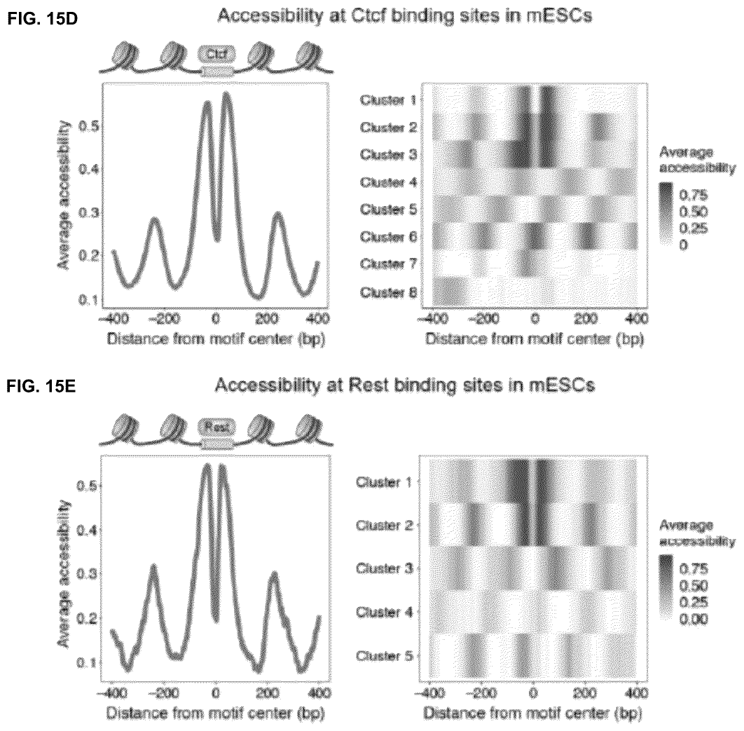

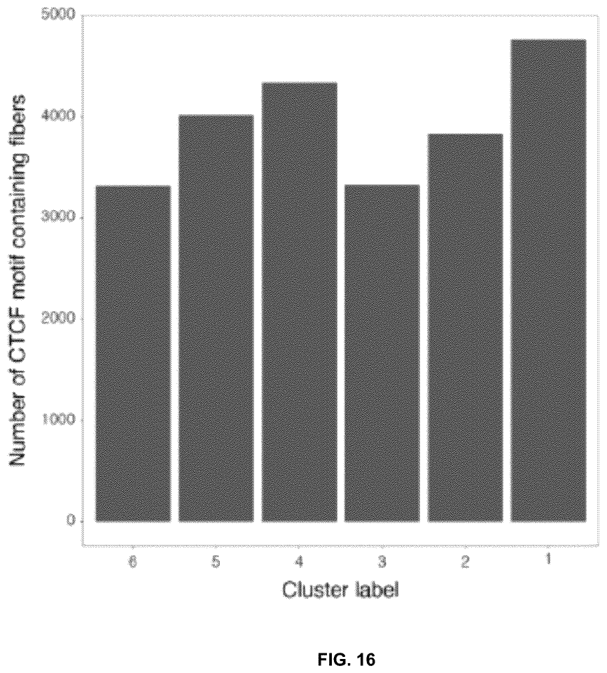

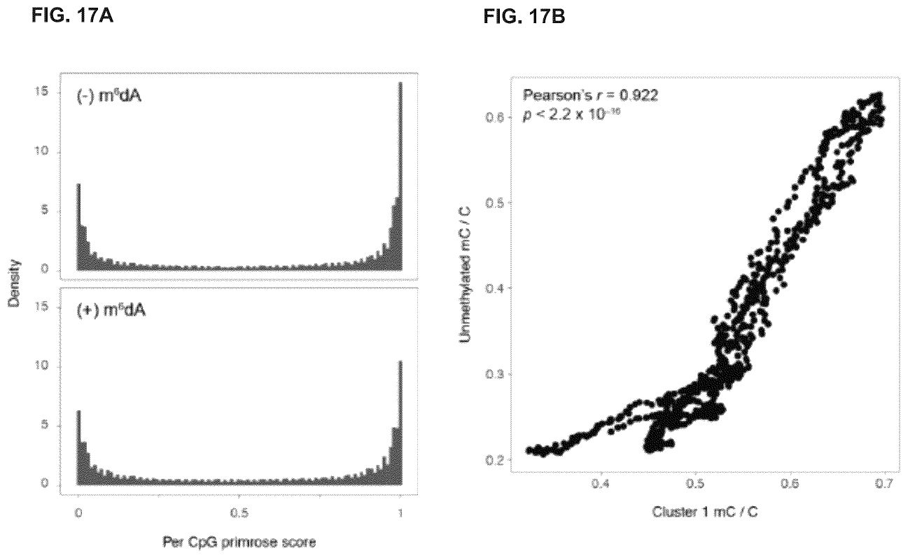

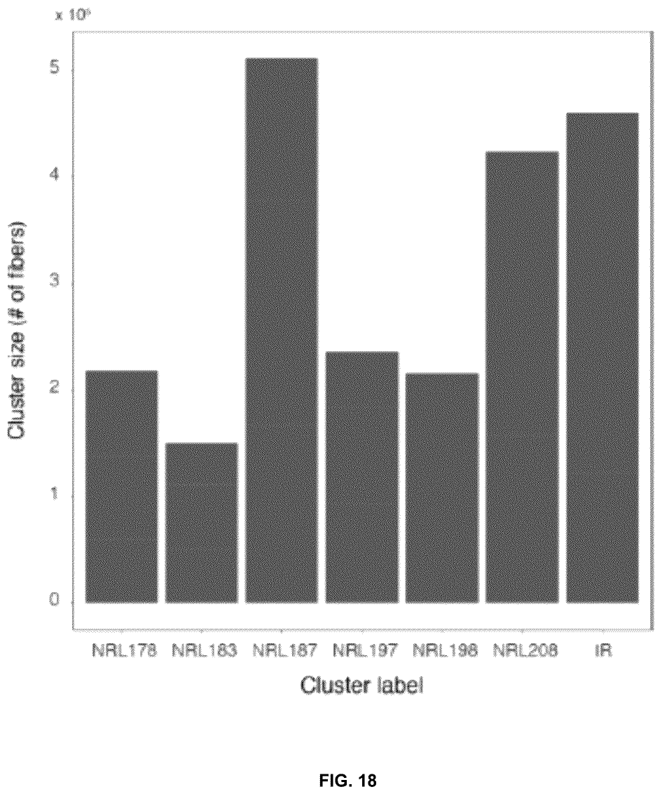

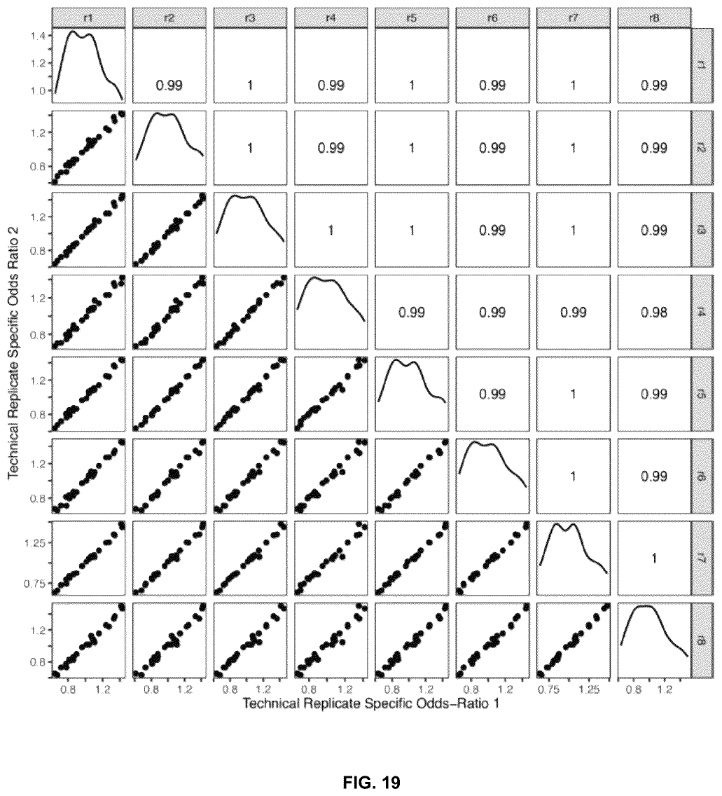

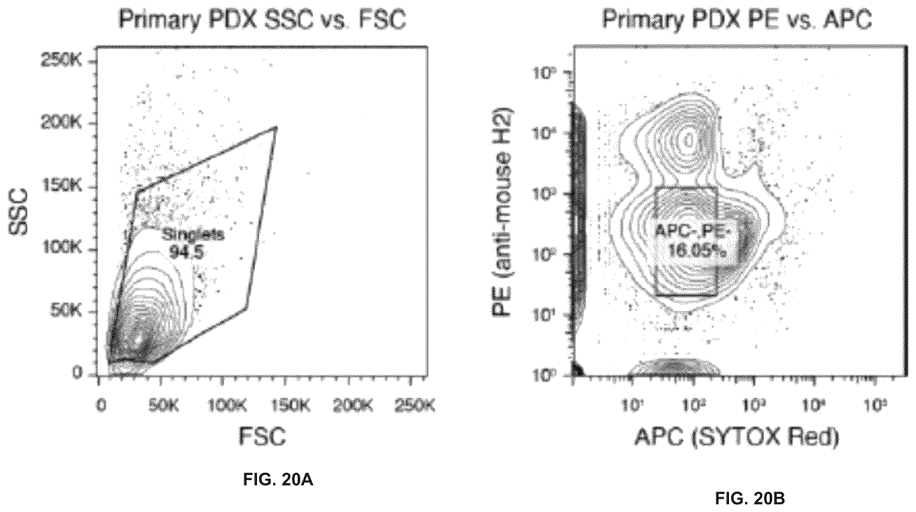

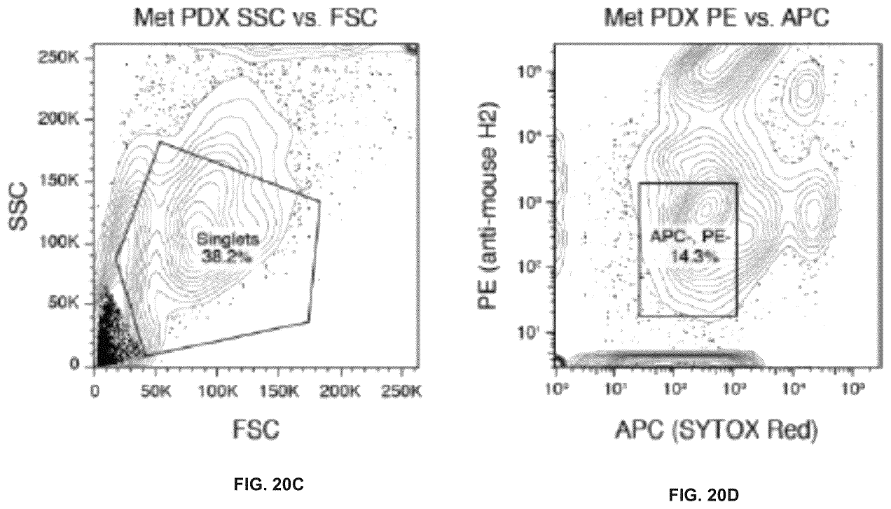

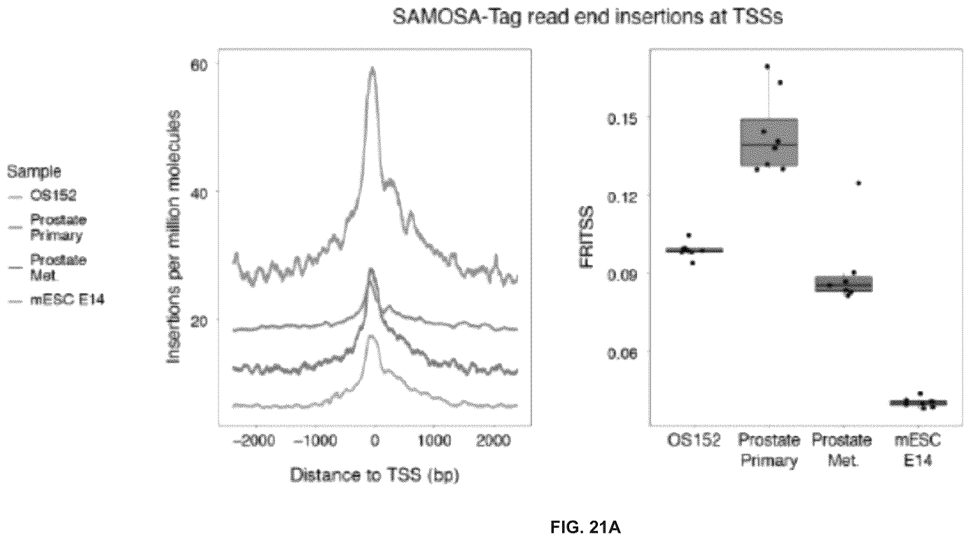

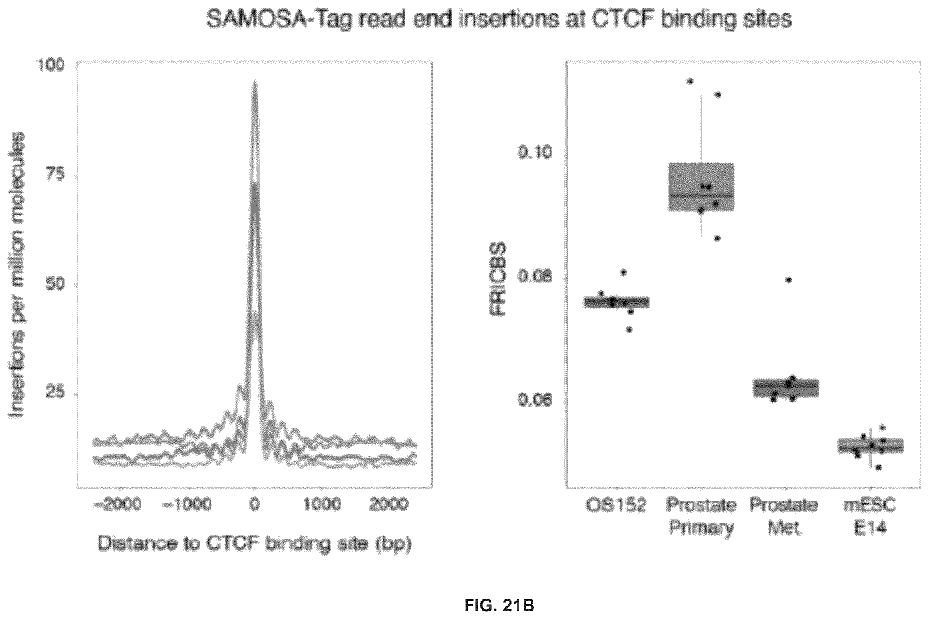

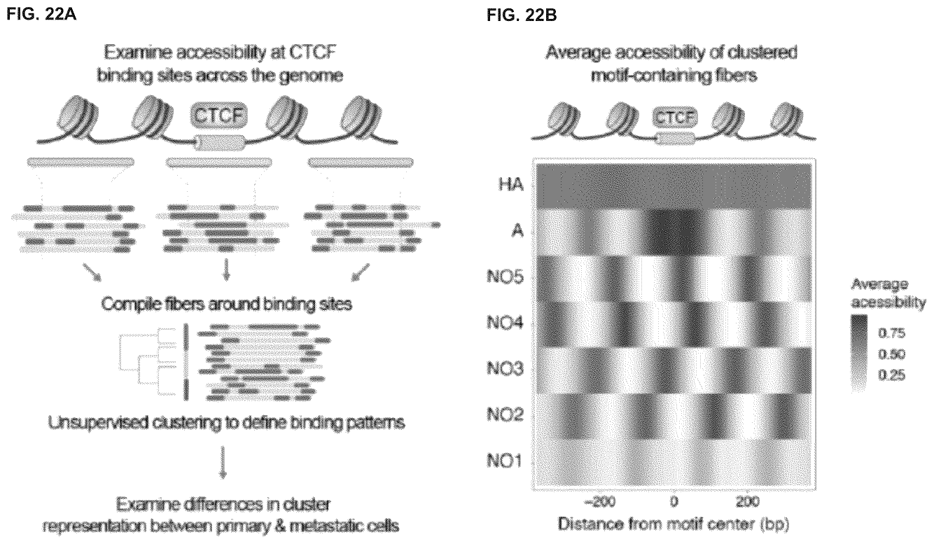

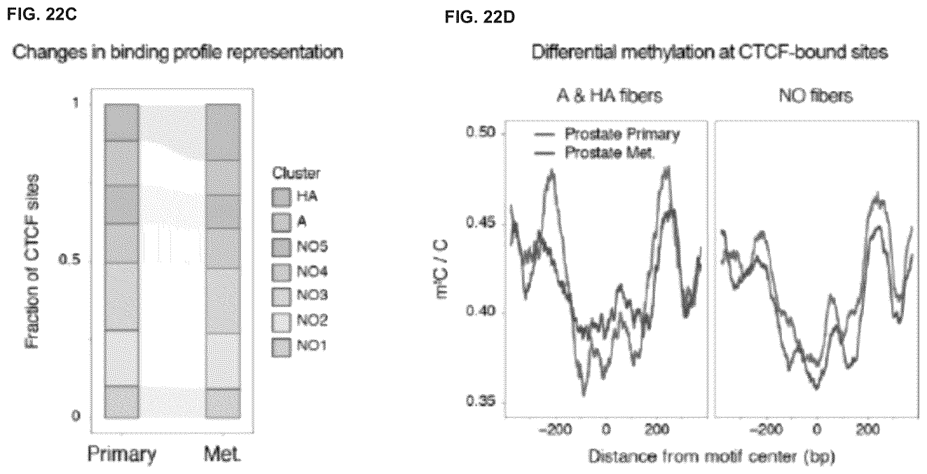

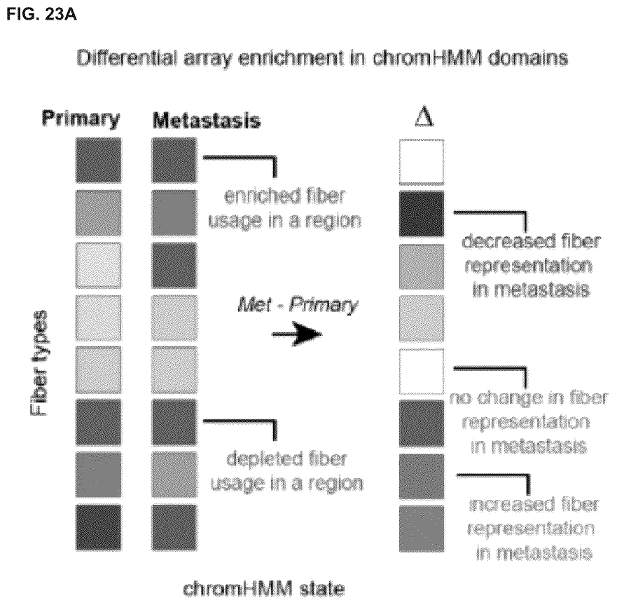

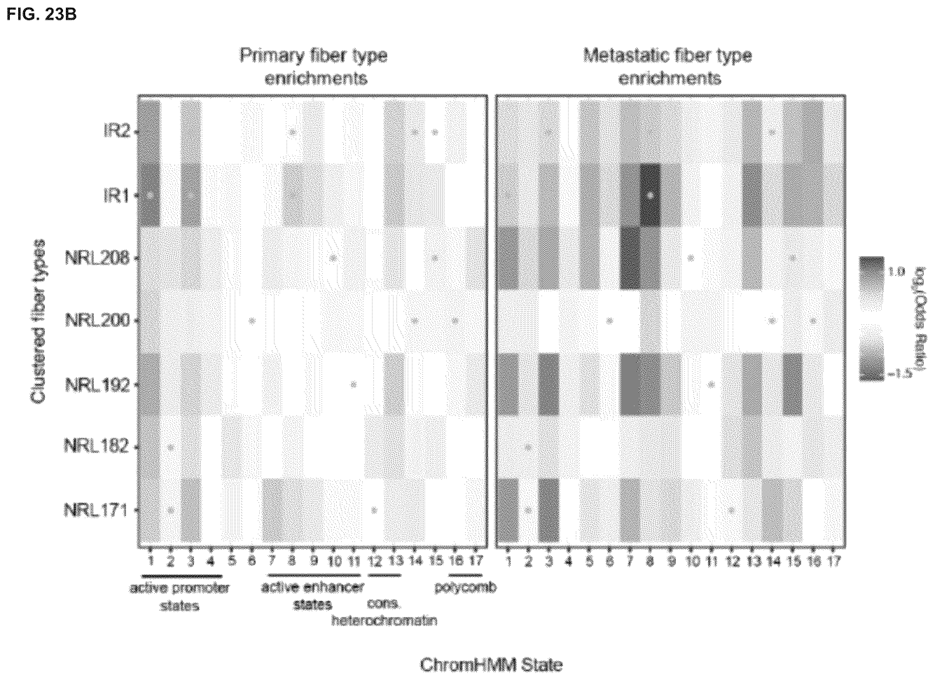

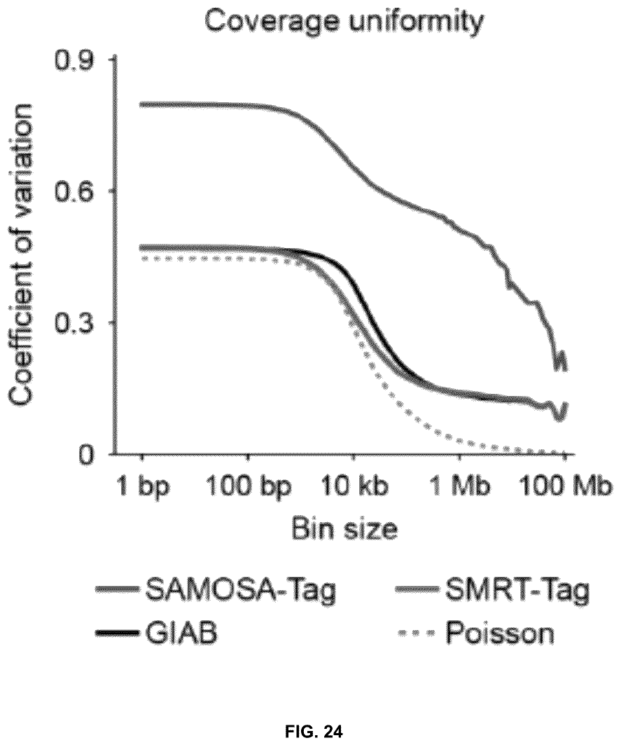

Tag concurrently profiles protein-DNA interactions and CpG methylation on single chromatin fibers. (FIG. 4A) Average SAMOSA (m6dA) accessibility and CpG methylation on 27,793 footprinted fibers from OS152 human osteosarcoma cells, centered at binding sites predicted from published U2OS ChIP-seq data³⁴. (FIG.4B) Visualization of m6dA signal for individual, clustered fibers centered at predicted CTCF motifs, reflecting different CTCF-occupied, accessible, and inaccessible states (800 molecules per cluster). (FIG. 4C) Average accessibility (left) and CpG methylation (right) for each of 6 clustered accessibility states around CTCF motifs. Window size is 750-nt for FIG.s 4A-4C. (FIG. 4D) Average primrose CpG methylation score for individual fibers as a function of density of CpG dinucleotides per kb. Molecules were binned into one of four bins, depending on CpG density and average primrose score. (FIG. 4E) Average accessibility of 7 different fiber types determined by Leiden clustering of single-fiber m⁶dA chromatin accessibility autocorrelation. Clusters stratify the entire genome by nucleosome repeat length (NRL ranging 178-208 nt) or irregularity (cluster IR). (FIG. 4F) Relative enrichment or depletion (Fisher's exact test) of individual fiber types for the same clusters as in FIG. 4E in each of the four binned states from d. All tests shown are statistically significant (p-values range from ˜0 to 2.41×10⁻⁵).

FIGS. 5A-5D are a series of plots and heat maps showing SAMOSA-Tag of patient-derived xenografts (PDXs) nominates global chromatin dysregulation in prostate cancer metastasis. (FIG. 5A) Overview of approach for SAMOSA-Tag of PDX models generated from primary and metastatic castration-resistant prostate tumors sampled from a single patient. Live, human cells were enriched from tumors explanted from PDX mice via fluorescence-assisted cell sorting (FACS). Six replicate SAMOSA-Tag reactions were performed using ˜30,000 nuclei each isolated from primary and metastatic PDXs. (FIG. 5B) Clustered fiber types detected in footprinted primary and metastatic chromatin fibers falling in one of 17 prostate-specific chromHMM states. Unsupervised Leiden clustering identified 7 fiber types—five regular clusters ranging in nucleosome repeat length (NRL) from 171-208 bp, and two irregular clusters. (FIG. 5C) Heatmap of effect-size estimated by logistic regression analysis to identify statistically significant differences in fiber type usage across chromHMM states. This analysis considered all six replicates from primary and metastatic cells. Red indicates fiber types enriched in metastasis, while blue indicates fiber types enriched in primary tumor. Grey dots mark non-significant (N.S.) results. (FIG. 5D) Speculative model of changes in single-molecule chromatin accessibility during prostate cancer progression based on PDX SAMOSA-Tag. Highly accessible, irregular chromatin fibers devoid of phased nucleosomes are enriched in metastatic cells suggestive of deranged activity of SWI/SNF remodelers, which are prime candidates for generating nucleosome-free/irregular single-molecule accessibility patterns. Chromatin state legends for c: active transcription start site (TssA), flanking transcription start site (TssFInk), upstream flanking transcription start site (TssFlnkU), downstream flanking transcription start site (TssFInkD), strong transcription (Tx), weak transcription (TxWk), genic enhancer (EnhG1 and EnhG2), active enhancer (EnhA1 and EnhA2), weak enhancer (EnhWk), zinc finger genes and repeats (ZNF/Rpts), heterochromatin (Het), bivalent/poised transcription start site (TssBiv), bivalent enhancer (EnhBiv), repressed polycomb (RepPC), and weak repressed polycomb (RepPCWk).

FIG. 6 shows the repair efficiency for a subset of the 62 conditions tested to optimize gap repair. Repair efficiency (defined as percent yield of product compared to input DNA by mass following exonuclease treatment) for 35 of the 62 conditions tested. A mixture of Phusion polymerase and Taq ligase was selected for gap repair as these provided the most consistently high repair efficiency across multiple experiments.

FIG. 7 is an example analytical gel trace for validating the size distribution of products for a subset of gap repair conditions. In addition to repair efficiency, we also validated that gap repair conditions did not appreciably change the size distribution of resulting libraries by gel electrophoresis. Shown here are analytical gel traces for six specific conditions tested in this study, including Phusion/Taq in multiple buffers.

FIGS. 8A-8F are a series of schematics and heat maps demonstrating the control experiments to establish multiplexing with SMRT-Tag. (FIG. 8A) Overview of genotype mixing experiment wherein gDNA from the HG003, HG004, and HG002 human trio were individually barcoded with one of 8 uniquely loaded transposomes, gap-repaired, and exo-treated prior to pooling for sequencing. (FIG. 8B) Heatmap of results from PacBio's lima demultiplexer, which annotates molecules with matching barcodes, versus those with mixed barcodes. Signal along the diagonal demonstrates minimal cross-contamination between barcodes/samples. (FIG. 8C) Percentage shared genotype across barcoded samples. HG002 (child) shares SNVs with HG003 and HG004 (parents), but HG003 and HG004 (parents) have minimal genotype overlap. This analysis considered all ‘private’ SNVs across HG003 and HG004. (FIG. 8D) Overview of experiment to validate pooled gap repair without pervasive barcode hopping wherein gDNA from one individual was barcoded with one of four different transposomes prior to pooled gap repair, exo digestion, and sequencing. (FIG. 8E) As in FIG. 8B but for pooled experiment in FIG. 8D. (FIG. 8F) Distributions of lima quality scores for barcoded molecules from FIG. 8D.

FIGS. 9A-9C are a series of plots showing the effect of Tn5 concentration, input amount, and temperature on tagmentation. (FIG. 9A) CCS fragment length distributions for various SMRT-Tag libraries constructed by varying Tn5 concentration (columns) and input amount (rows) at 55° C. (red curves) and 37° C. (blue curves). (FIG. 9B) Effect of varying transposome amount keeping input DNA quantity fixed at 40 ng. (FIG. 9C) Quantification of mean, mode, median, and standard deviation (SD) for each sequenced library as a function of transposome dilution factor.

FIGS. 10A-10F are a series of plots, graphs and heatmaps showing the

benchmarking high coverage HG002 SMRT-Tag and ligation-based PacBio libraries against GIAB and CpG methylation standards. (FIG. 10A) Precision, recall, and F1 scores for Deep Variant single nucleotide variant (SNV) and insertion/deletion (indel) calls from high-coverage SMRT-Tag libraries and coverage-matched, ligation-based PacBio data compared against GIAB truth sets. (FIG. 10B) Precision as a function of recall for SNVs and indels for SMRT-Tag and ligation-based PacBio data benchmarked against GIAB truth sets. Performance characteristics (FIG. 10C) in aggregate and (FIG. 10D) binned by structural variant (SV) size for pbsv calls from SMRT-Tag and coverage-matched, ligation-based PacBio data benchmarked against the GIAB SV call set. Comparison of SMRT-Tag primrose CpG methylation against (FIG. 10E) bisulfite and (FIG. 10F) ligation-based PacBio data.

FIGS. 11A-11B demonstrate the performance of SMRT-Tag in difficult-to-genotype regions and as a function of sequencing depth. (FIG. 11A) Deep Variant precision/recall curves for SNV (red) and indel (blue) calls in challenging genomic regions, including segmental duplications, tandem repeats, homopolymers, and the MHC locus, for high coverage SMRT-Tag data (solid) versus coverage-matched, ligation-based PacBio data27 (dashed). (FIG. 11B) Composite F1 score for SMRT-Tag (closed circles) versus GIAB data (open squares) as a function of sequencing depth, for SNV (red) and indel (blue) calls.

FIG. 12 demonstrates the genome-wide correlation of OS152 SAMOSA-Tag and ATAC-seq accessibility. SAMOSA-Tag methyltransferase and ATAC-seq transposase accessibility are positively correlated (Pearson's r=0.576, p<2.2×10⁻¹⁶).

FIGS. 13A-13B show examples of SAMOSA-Tag coverage and signal plotted with ATAC-seq data for copy-number neutral (SMAD3; FIG. 13B) and copy-number loss (GRIN2A; FIG. 13A) loci.

FIGS. 14A-14C demonstrate the subtle insertional preference at transcription start sites and CTCF motifs in OS152 SAMOSA-Tag experiments. Metaplots of insertions per million sequenced OS152 SAMOSA-Tag molecules in 5-kb windows centered at (FIG. 14A) hg38 transcription start sites (TSSs) and (FIG. 14B) U2OS ChIP-seq-backed CTCF binding sites. Signal was smoothed using a 100-nt running mean. (FIG. 14C) Boxplots of fraction of insertions in TSS (FRITSS) and in CTCF binding sites (FRICBS) across all eight replicate experiments.

FIGS. 15A-15E are a series of schematics, plots and heatmaps demonstrating that SAMOSA-Tag generalizes to different cell types, and can be performed in situ or ex situ, and can footprint factors other than CTCF/Ctcf. (FIG. 15A) Fragment length distributions, (FIG. 15B) mean single molecule m⁶dA accessibility, and (FIG. 15C) sizes of EcoGII methylase-inaccessible footprints in mouse embryonic stem cells (mESCs) for SAMOSA-Tag performed in situ (tagmentation of intact nuclei after EcoGII treatment; purple) and ex situ (tagmentation of DNA extracted from nuclei after EcoGII treatment; green). (FIG. 15D) In situ mESC SAMOSA-Tag molecules were clustered into 8 single-molecule accessibility patterns around Ctcf sites predicted using ChIP-seq data. (FIG. 15E) As in FIG. 15D but for Nrsf/Rest centered at sites predicted using published ChIP-seq data⁵³.

FIG. 16 is a graph demonstrating the cluster sizes resulting from Leiden

clustering of single-molecule accessibility patterns surrounding predicted CTCF sites. Cluster labels match FIGS. 4B, 4C.

FIGS. 17A-17B are plots demonstrating that m⁶dA footprinting does not appreciably impact CpG methylation detection. (FIG. 17A) Distribution of per-CpG primrose scores (50,000 sampled CpGs per experiment) for negative control experiments where EcoGII was omitted (no m⁶dA; top) and SAMOSA-Tag experiments (bottom). (b) Correlation of average CpG methylation from SAMOSA-Tag molecules with detectable modA signal (cluster 1, FIGS. 4B, 4C) versus without appreciable adenine methylation around predicted CTCF sites (Pearson's r=0.922, p<2.2×10⁻¹⁶).

FIG. 18 is a graph demonstrating fiber type cluster sizes resulting from Leiden clustering of SAMOSA-Tag accessibility autocorrelation. Cluster labels match FIGS. 4E, 4F.

FIG. 19 is a series of plots demonstrating SAMOSA-Tag fiber enrichments in differential CpG content/CpG methylation bins are technically reproducible. Matrix of scatter plots with Pearson's r correlation values across each of eight replicate OS152 SAMOSA-Tag experiments. FIGS. 20A-20D are plots showing the FACS gating strategy for PDX live-dead/human-mouse sorts. (FIGS. 20A, 20B) Primary prostate tumor PDX sorts. (FIGS. 20C, 20D) Metastatic prostate tumor PDX sorts.

FIGS. 21A-21B are a series of plots and graphs showing a comparison of insertion preference in PDX and cell line SAMOSA-Tag experiments. Insertion preference (left) and FRITSS scores (right) at (FIG. 21A) TSSs and (FIG. 21B) ChIP-backed CTCF binding sites for cell line (OS152 and mESC E14) and PDX SAMOSA-Tag data.

FIGS. 22A-22D are a series of schematics, plots, heatmaps demonstrating differential single-molecule chromatin accessibility at CTCF sites in primary and metastatic PDX prostate cancer cells. (FIG. 22A) Overview of framework for analyzing CTCF motif accessibility on individual chromatin fibers from SAMOSA-Tag of primary and metastatic prostate tumor PDXs. (FIG. 22B) Unsupervised Leiden clustering of single-molecule chromatin accessibility centered at CTCF motifs identified 7 different occupancy states (differentially colored): nucleosome occupied (NO) states with varying nucleosomal registers around the CTCF motif (NO1-NO5), and 2 accessible states termed ‘A’ (with characteristically phased nucleosomes flanking occupied CTCF motifs) and ‘HA’ (hyper-accessibility of the entire 750-nt window is accessible to EcoGII). (FIG. 22C) Alluvial plot of shifts in occupancy state distribution between primary tumor and metastasis with notable increase in cluster HA and decrease in cluster A in metastatic cells. (FIG. 22D) Co-measurement of m⁶dA accessibility and CpG methylation in fibers of type A and HA (left) and NO (right). In metastatic cells compared to primary tumor, while accessible/hyper-accessible CTCF motifs are slightly hypermethylated, CTCF sites in the NO state have this effect reversed with subtle hypomethylation.

FIGS. 23A-23B are a series of schematics and heatmaps demonstrating differential and per-sample fiber-type enrichments in primary and metastatic PDXs. (FIG. 23A) Overview of the approach for computing a statistic “delta” (66 ) which aims to quantify differential epresentation of fiber types in specific chromHMM domains across the human epigenome in a statistically rigorous manner. Beginning with computed per-domain enrichments in each sample and associated counts, we compute an estimated effect-size (Δ) and associated q values using a customized logistic regression analysis and visualize these data in heatmap form with different color scales. (FIG. 23B) Fisher's exact test results for each sample (primary vs. met) for clustered fiber types (signal averages shown in FIG. 5B). Red indicates an overrepresentation of that fiber type (y-axis) within the domain (x-axis); blue indicates a depletion of a fiber type within a domain. Grey dots designate tests that are not significant (N.S.). Chromatin state legends: 1: TSS, 2: TSS Flank, 3: TSS Flank Upstream, 4: TSS Flank Downstream, 5: Transcribed region, 6: Weakly transcribed region, 7: Genic enhancer 1, 8: Genic enhancer 2, 9: Active enhancer 1, 10: Active enhancer 2, 11: Weak enhancer, 12: KRAB zinc finger/repetitive region, 13: Constitutive heterochromatin, 14: Bivalently-marked TSS, 15: Bivalently-marked enhancer, 16: Polycomb repressed, 17: Weakly polycomb repressed.

FIG. 24 is a plot showing coverage uniformity of tagmentation-and ligation-based libraries. Rarefaction curves demonstrating differences in coverage uniformity at varying window sizes across the genome for SAMOSA-Tag (red), SMRT-Tag (blue), ligation-based PacBio data (black) compared against a random control based on Poisson sampling of reads from the human genome (dashed).

### DETAILED DESCRIPTION

While low-input sequencing protocols are available, they typically rely on PCR amplification, which erases modified bases and may introduce biases. This obstacle has limited the primary use of SMS to genome assembly and medical genetics, precluding analyses of rare clinical samples and post-mitotic cell populations, single cells, and microorganisms.

This disclosure is based on, in part, methods that are PCR-free. Particular examples include: (i) single-molecule real time sequencing by tagmentation (SMRT-Tag) for assaying the genome and epigenome, and (ii) SAMOSA-Tag, which adds a concurrent channel for mapping chromatin structure. SMRT-Tag accurately detected genetic and epigenetic variants from as little as 40 ng of DNA. SAMOSA-Tag maps of single-fiber CTCF and nucleosome occupancy and CpG methylation uncovered metastasis-associated global chromatin deregulation in technically challenging patient-derived prostate cancer xenografts. These results extend tagmentation to PacBio library preparation and have the potential to enable sensitive, scalable, and cellularly resolved single-molecule genomics.

Simultaneous transposition of sequencing adaptors and template DNA fragmentation (i.e., ‘tagmentation’) using hyperactive transposase poses an attractive solution to this problem¹⁴. The reduced input requirement and workflow complexity of Tn5-based short-read library preparation has transformed bulk genome, epigenome, and transcriptome profiling¹⁵⁻¹⁷ and enabled single-cell and spatial monoplex¹⁸⁻²⁰ and multiomic sequencing²¹⁻²³.

### Single Molecule Sequencing of DNA Fragments

Single molecule sequencing often involves the optical observation of the polymerase process during the process of nucleotide incorporation, for example, observation of the enzyme-DNA complex. During this process, there are generally two or more observable phases. For example, where a terminal-phosphate labeled nucleotide is used and the enzyme-DNA complex is observed, there is a bright phase during the steps where the label is incorporated with (bound to) the polymerase enzyme, and a dark phase where the label is not incorporated with the enzyme. For the purposes of this disclosure, both the dark phase and the bright phase are generally referred to as observable phases, because the characteristics of these phases can be observed.

Whether a phase of the polymerase reaction is bright or dark can depend, for example, upon how and where the components of the reaction are labeled and also upon how the reaction is observed. For example, the phase of the polymerase reaction where the nucleotide is bound can be bright where the nucleotide is labeled on its terminal phosphate. However, where there is a quenching dye associated with the enzyme or template, the bound state may be quenched, and therefore be a dark phase. Analogously, in a ZMW, the release of the terminal phosphate may result in a dark phase, whereas in other systems, the release of the terminal phosphate may be observable, and therefore constitute a bright phase.

At a contrast, Single Molecule Real Time (SMRT) sequencing relies on an ultra-processive DNA polymerase and specialized optics to track polymerase-mediated base addition in real time. Central to this process is the zero-mode waveguide (ZMW), a nanowell structure with a volume of ˜20 zeptoliters (˜2×10⁻¹² liters) and a diameter smaller than specific wavelengths of light. Double stranded DNA molecules between 2-25 kb in size are first converted into templates for rolling circle amplification by ligating annealed hairpin adapters (“SMRT adapters”) to DNA ends. Templates are then annealed with engineered sequencing polymerases (originally derived from bacteriophage polymerase Phi29) and single polymerase/DNA complexes anchored to the bottom of each ZMW. Complexes are illuminated from below by a laser and nucleotides with base-specific fluorescent dyes conjugated to their terminal phosphate groups are added to initiate polymerization. Base incorporation by the polymerase momentarily holds the fluorescent dye in the laser path, triggering fluorescent emission of photons that are captured within the ZMW and detected before the linked pyrophosphate is cleaved to form the phosphodiester bond. This reaction can then continue for hundreds of thousands of bases (on the order of ˜300kb), producing extremely long polymerase reads that are effectively re-reads (“subreads”) of each strand of the original library molecule due to the rolling circle process. Subreads are merged computationally, taking advantage of the randomized nature of incorporation errors, to produce a highly accurate circular consensus read per single molecule (“CCS read”).

On the latest PacBio instruments, flow cells (“SMRTcells”) contain between 8M-25M ZMWs each, generating multiple millions of CCS reads per run (˜2-3M on the Sequel II, 4-6M on the newer Revio), with nearly all (>90%) meeting the HiFi criteria (per-base accuracy >99.9%). The high single-molecule accuracy and long read lengths of HiFi sequencing have made it the go-to favorite for producing reference grade genome assemblies. For example, the recently completed telomere-to-telomere human reference genome relied heavily on HiFi reads to close assembly gaps, while using nanopore reads for long-distance scaffolding. Further, native sequencing without PCR significantly reduces GC biases, and the SMRT sequencing polymerase is not affected by highly repetitive sequence content as in SBS.

Critically, SMRT sequencing is highly sensitive to nucleotide modifications—a property which has been leveraged by methyltransferase footprinting methods for native methylation detection. When the SMRT polymerase cognates against bases with epigenetic modifications, it temporarily pauses extending the duration between the previous base incorporation and the next. This time interval, called the inter-pulse duration (IPD), along with the width of the subsequent fluorescent pulse (pulse width, PW) are two highly informative kinetic parameters produced per base sequenced that uniquely characterize the epigenetic modification and the surrounding sequence context. While earlier studies deemed changes in PW and IPD too subtle for detection, machine learning models, particularly convolutional and recurrent neural networks, trained on these kinetic parameters using whole genome amplified (unmodified, negative control) and methyltransferase treated (modified, positive control) DNA can accurately detect m⁶dA and m⁵dC with single base and single molecule resolution. Single molecule accessibility techniques have therefore benefitted from advances in modification detection to efficiently call exogenous m⁶dA marks and resolve stretches of accessible sequence.

Third-generation, single-molecule long-read sequencing (SMS) technologies deliver highly accurate genomic and epigenomic readouts of kilobase to megabase-length nucleic acid templates. SMS has facilitated the characterization of previously intractable structural variants and repetitive regions, assembly of a gapless human genome, and high-resolution functional genomic profiling of both DNA and RNA. The multimodality of SMS has also been exploited by single molecule chromatin profiling methods such as the single-molecule adenine methylated oligonucleosome sequencing assay (SAMOSA), Fiber-seq, directed methylation long-read sequencing (DiMelo-seq), nanopore sequencing of nucleosome occupancy through methylation (NanoNOMe), and others. These approaches establish a paradigm for simultaneously measuring functional genomic information (e.g. histone/transcription factor-DNA interactions) as separate SMS “channels” along with primary sequence and endogenous epigenetic marks.

In certain embodiments, single molecule sequencing is conducted in order to provide high-resolution, high-throughput sequence information. Template-dependent single-molecule sequencing-by-synthesis is conducted using optically-labeled nucleotides. The sequencing can be performed in certain instances by attaching the nucleic acids to a surface that is designed to enhance optical signal detection. An example of a surface is an epoxide surface coated onto glass or fused silica. Nucleic acids are easily attached to epoxide or epoxide derivatives. In certain embodiments, the attachment is direct amine attachment. Nucleic acids can be purchased with a 5′ or 3′ amine, or terminal transferase can be used to introduce a terminal amine for attachment to the epoxide ring. Alternatively, epoxide surfaces can be derivatized for nucleic acid attachment. For example, the surface can incorporate streptavidin, which binds to biotinylated nucleic acids. Alternative surfaces include polyelectrolyte multilayers as described in Braslavasky, et al., PNAS 100:3960-64 (2003). Essentially, any surface that has reduced native fluorescence and is amenable to attachment of oligonucleotides is useful.

Single molecule sequence is advantageously performed using optically-detectable labels. Especially preferred are fluorescent labels, including fluorescein, rhodamine, derivatized rhodamine dyes, such as TAMRA, phosphor, polymethadine dye, fluorescent phosphoramidite, texas red, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA, or a derivative or modification of any of the foregoing.

A capture step prior to sequencing may be conducted. Any suitable hybrid capture method. For example, capture can occur in solution, on beads (polystyrene beads), in a column (such as a chromatography column), in a gel (such as a polyacrylamide gel), or directly on the surface to be used for sequencing. An array of support-bound capture oligos can be used to hybridize specifically to a target sequence. Additionally, chromatography-based capture techniques are useful. For example, ion exchange chromatography, HPLC, gas chromatography, and gel-based chromatography all are useful. In one embodiment, gel-based capture is used in order to achieve sequence-specific capture. Using this method, multiple different sequences are captured simultaneously using immobilized probes in the gel. The target sequences are isolated by removing portions of the gel containing them and eluting target from the gel portions for sequencing.

### Tagmentation

As used herein, the term “tagmentation” refers to the modification of DNA by a transposome complex comprising transposase enzyme complexed with adaptors comprising transposon end sequence. Tagmentation results in the simultaneous fragmentation of the DNA and ligation of the adaptors to the 5′ ends of both strands of duplex fragments. Following a purification step to remove the transposase enzyme, additional sequences can be added to the ends of the adapted fragments, for example by PCR, ligation, or any other suitable methodology known to those of skill in the art. The method of can use any transposase that can accept a transposase end sequence and fragment a target nucleic acid, attaching a transferred end, but not a non-transferred end. A “transposome” is comprised of at least a transposase enzyme and a transposase recognition site. In some such systems, termed “transposomes”, the transposase can form a functional complex with a transposon recognition site that is capable of catalyzing a transposition reaction. The transposase or integrase may bind to the transposase recognition site and insert the transposase recognition site into a target nucleic acid in a process sometimes termed “tagmentation”. In some such insertion events, one strand of the transposase recognition site may be transferred into the target nucleic acid. In standard sample preparation methods, each template contains an adaptor at either end of the insert and often a number of steps are required to both modify the DNA or RNA and to purify the desired products of the modification reactions. These steps are performed in solution prior to the addition of the adapted fragments to a flowcell where they are coupled to the surface by a primer extension reaction that copies the hybridized fragment onto the end of a primer covalently attached to the surface. These ‘seeding’ templates then give rise to monoclonal clusters of copied templates through several cycles of amplification. The number of steps required to transform DNA into adaptor-modified templates in solution ready for cluster formation and sequencing can be minimized by the use of transposase mediated fragmentation and tagging. In some embodiments, transposon based technology can be utilized for fragmenting DNA, for example as exemplified in the workflow for Nextera DNA sample preparation kits (Illumina, Inc.) wherein genomic DNA can be fragmented by an engineered transposome that simultaneously fragments and tags input DNA (“tagmentation”) thereby creating a population of fragmented nucleic acid molecules which comprise unique adapter sequences at the ends of the fragments. Some embodiments can include the use of a hyperactive Tn5 transposase and a Tn5-type transposase recognition site (Goryshin and Reznikoff, J. Biol. Chem., 273:7367 (1998)), or MuA transposase and a Mu transposase recognition site comprising RI and R2 end sequences (Mizuuchi, K., Cell, 35:785, 1983; Savilahti, H, et al., EMBO J., 14:4893, 1995). An exemplary transposase recognition site that forms a complex with a hyperactive Tn5 transposase (e.g., EZ-Tn5 Transposase, Epicentre Biotechnologies, Madison, Wis.). More examples of transposition systems that can be used with certain embodiments provided herein include Staphylococcus aureusTn552 (Colegio et al., J. Bacteriol., 183:2384-8, 2001; Kirby C et al., Mol. Microbiol., 43:173-86, 2002), Tyl (Devine & Boeke, Nucleic Acids Res., 22:3765-72, 1994 and International Publication WO 95/23875), Transposon Tn7 (Craig, N L, Science. 271: 1512, 1996; Craig, N L, Review in: Curr Top Microbiol Immunol., 204:27-48, 1996), Tn10 and IS10 (Kleckner N, et al., Curr Top Microbiol Immunol., 204:49-82, 1996), Mariner transposase (Lampe D J, et al., EMBO J., 15:5470-9, 1996), Tel (Plasterk R H, Curr. Topics Microbiol. Immunol., 204:125-43, 1996), P Element (Gloor, G B, Methods Mol. Biol., 260:97 114, 2004), Tn3 (Ichikawa & Ohtsubo, J Biol. Chem. 265:18829-32, 1990), bacterial insertion sequences (Ohtsubo & Sekine, Curr. Top. Microbiol. Immunol. 204:1-26, 1996), retroviruses (Brown, et al., Proc Natl Acad Sci USA, 86:2525-9, 1989), and retrotransposon of yeast (Boeke & Corces, Annu Rev Microbiol. 43:403-34, 1989). More examples include IS5, Tn10, Tn903, IS911, and engineered versions of transposase family enzymes (Zhang et al., (2009) PLoS Genet. 5: e1000689. Epub 2009 Oct. 16; Wilson C. et al (2007) J. Microbiol. Methods 71:332-5). Briefly, a “transposition reaction” is a reaction wherein one or more transposons are inserted into target nucleic acids at random sites or almost random sites. Essential components in a transposition reaction are a transposase and DNA oligonucleotides that exhibit the nucleotide sequences of a transposon, including the transferred transposon sequence and its complement (i.e., the non-transferred transposon end sequence) as well as other components needed to form a functional transposition or transposome complex. The DNA oligonucleotides can further comprise additional sequences (e.g., adaptor or primer sequences) as needed or desired. Briefly, in vitro transposition can be initiated by contacting a transposome complex and a target DNA. Exemplary transposition procedures and systems that can be readily adapted for use with the transposases of the present disclosure are described, for example, in WO 10/048605; US 2012/0301925; US 2013/0143774, each of which is incorporated herein by reference in its entirety. The adapters that are added to the 5′ and/or 3′ end of a nucleic acid can comprise a universal sequence. A universal sequence is a region of nucleotide sequence that is common to, i.e., shared by, two or more nucleic acid molecules. Optionally, the two or more nucleic acid molecules also have regions of sequence differences. Thus, for example, the 5′ adapters can comprise identical or universal nucleic acid sequences and the 3′0 adapters can comprise identical or universal sequences. A universal sequence that may be present in different members of a plurality of nucleic acid molecules can allow the replication or amplification of multiple different sequences using a single universal primer that is complementary to the universal sequence. Some universal primer sequences used in examples presented herein include the V2.A14 and V2.B15 Nextera™ sequences. However, it will be readily appreciated that any suitable adapter sequence can be utilized in the methods and compositions presented herein. For example, Tn5 Mosaic End Sequence A14 (Tn5MEA) and/or Tn5 Mosaic End Sequence B15 (Tn5MEB) can be used in the methods provided herein.

In certain embodiments, the transposase is a hyperactive transposase. In certain embodiments, the hyperactive transposase is prokaryotic, eukaryotic or proteases.In certain embodiments, the prokaryotic hyperactive transposases comprise Tn5, Tn5 embodiments, a Tn5 mutant comprises one or more mutations. In certain embodiments, the Tn5 mutant comprises an R27S, an E54K, an L372P substitution or combinations thereof. In certain embodiments, a Tn5 derivative is linked to an epitope comprising protein A, nanobodies, biotin, streptavidin, protein G, FK-binding protein, beads or combinations thereof. In certain embodiments, the protease transposases comprise casposases, Cas9 or combinations thereof. In certain embodiments, the eukaryotic transposases comprise retrotransposons (class I transposons), class II transposons or miniature inverted-repeat transposable elements (MITEs, or class III transposons). In certain embodiments, the eukaryotic transposases comprise Sleeping Beauty transposon system (SBTS), piggyBac (PB) transposons, Hermes transposons or combinations thereof.

### Barcodes

Generally, a barcode can include one or more nucleotide sequences that can be used to identify one or more particular nucleic acids. The barcode can be an artificial sequence or can be a naturally occurring sequence generated during transposition, such as identical flanking genomic DNA sequences (g-codes) at the end of formerly juxtaposed DNA fragments. In some embodiments, a barcode is an artificial sequence that is non-natural to the target nucleic acid and is used to identify the target nucleic acid or determine the contiguity information of the target nucleic acid.

A barcode can comprise at least about 1, 2, 3, 4, 5, 6, 7, 8, 9, 10, 11, 12, 13, 14, 15, 16, 17, 18, 19, 20 or more consecutive nucleotides. In some embodiments, a barcode comprises at least about 10, 20, 30, 40, 50, 60, 70 80, 90, 100 or more consecutive nucleotides. In some embodiments, at least a portion of the barcodes in a population of nucleic acids comprising barcodes is different. In some embodiments, at least about 10%, 20%, 30%, 40%, 50%, 60%, 70%, 80%, 90%, 95%, 99% of the barcodes are different. In more such embodiments, all of the barcodes are different. The diversity of different barcodes in a population of nucleic acids comprising barcodes can be randomly generated or non-randomly generated.

In some embodiments, a transposon sequence comprises at least one barcode. In some embodiments, such as transposomes comprising two non-contiguous transposon sequences, the first transposon sequence comprises a first barcode, and the second transposon sequence comprises a second barcode. In some embodiments, a transposon sequence comprises a barcode comprising a first barcode sequence and a second barcode sequence. In some of the foregoing embodiments, the first barcode sequence can be identified or designated to be paired with the second barcode sequence. For example, a known first barcode sequence can be known to be paired with a known second barcode sequence using a reference table comprising a plurality of first and second bar code sequences known to be paired to one another.

In another example, the first barcode sequence can comprise the same sequence as the second barcode sequence. In another example, the first barcode sequence can comprise the reverse complement of the second barcode sequence. In some embodiments, the first barcode sequence and the second barcode sequence are different. The first and second barcode sequences may comprise a bi-code.

In some embodiments of compositions and methods described herein, barcodes are used in the preparation of template nucleic acids. As will be understood, the vast number of available barcodes permits each template nucleic acid molecule to comprise a unique identification. Unique identification of each molecule in a mixture of template nucleic acids can be used in several applications. For example, uniquely identified molecules can be applied to identify individual nucleic acid molecules, in samples having multiple chromosomes, in genomes, in cells, in cell types, in cell disease states, and in species, for example, in haplotype sequencing, in parental allele discrimination, in metagenomics sequencing, and in sample sequencing of a genome.

### Target Nucleic Acids

A target nucleic acid can include any nucleic acid of interest. Target nucleic acids can include DNA, RNA, peptide nucleic acid, morpholino nucleic acid, locked nucleic acid, glycol nucleic acid, threose nucleic acid, mixed samples of nucleic acids, polyploidy DNA (i.e., plant DNA), mixtures thereof, and hybrids thereof. In certain embodiments, genomic DNA is used as the target nucleic acid. In certain embodiments, cDNA, mitochondrial DNA or nucleus DNA is used.

A target nucleic acid can comprise any nucleotide sequence. In some embodiments, the target nucleic acid comprises homopolymer sequences. A target nucleic acid can also include repeat sequences. Repeat sequences can be any of a variety of lengths including, for example, 2, 5, 10, 20, 30, 40, 50, 100, 250, 500 or 1000 nucleotides or more. Repeat sequences can be repeated, either contiguously or non-contiguously, any of a variety of times including, for example, 2, 3, 4, 5, 6, 7, 8, 9, 10, 15 or 20 times or more.

In some embodiments, the target nucleic acid is a single target nucleic acid. Other embodiments can utilize a plurality of target nucleic acids. In such embodiments, a plurality of target nucleic acids can include a plurality of the same target nucleic acids, a plurality of different target nucleic acids where some target nucleic acids are the same, or a plurality of target nucleic acids where all target nucleic acids are different. Embodiments that utilize a plurality of target nucleic acids can be carried out in multiplex formats so that reagents are delivered simultaneously to the target nucleic acids, for example, in one or more chambers or on an array surface. In some embodiments, the plurality of target nucleic acids can include substantially all of a particular organism's genome. The plurality of target nucleic acids can include at least a portion of a particular organism's genome including, for example, at least about 1%, 5%, 10%, 25%, 50%, 75%, 80%, 85%, 90%, 95%, or 99% of the genome. In particular embodiments the portion can have an upper limit that is at most about 1%, 5%, 10%, 25%, 50%, 75%, 80%, 85%, 90%, 95%, or 99% of the genome.

In certain embodiments, target nucleic acids are from a single cell. In certain embodiments, the target nucleic acids are from a single a cell nucleus.

Target nucleic acids can be obtained from any source. For example, target nucleic acids may be prepared from nucleic acid molecules obtained from a single organism or from populations of nucleic acid molecules obtained from natural sources that include one or more organisms. Sources of nucleic acid molecules include, but are not limited to, organelles, cells, tissues, organs, organisms, single cell, or a single organelle. Cells that may be used as sources of target nucleic acid molecules may be prokaryotic (bacterial cells, for example, Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces genera); archeaon, such as crenarchaeota, nanoarchaeota or euryarchaeotia; or eukaryotic such as fungi, (for example, yeasts), plants, protozoans and other parasites, and animals (including insects (for example, Drosophila spp.), nematodes (e.g., Caenorhabditis elegans), and mammals (for example, rat, mouse, monkey, non-human primate and human).

In addition, in some embodiments, target nucleic acids and/or template nucleic acids can be highly purified, for example, nucleic acids can be at least about 70%, 80%, 90%, 95%, 96%, 97%, 98%, 99%, or 100% free from contaminants before use with the methods provided herein. In some embodiments, it is beneficial to use methods known in the art that maintain the quality and size of the target nucleic acid, for example isolation and/or direct transposition of target DNA may be performed using agarose plugs. Transposition can also be performed directly in cells, with population of cells, lysates, and non-purified DNA.

In some embodiments, target nucleic acid can be from a single cell. In some embodiments, target nucleic acid can be from formalin fixed paraffin embedded (FFPE) tissue sample. In some embodiments, target nucleic acid can be cross-linked nucleic acid. In some embodiments, the target nucleic acid can be cross-linked to nucleic acid. In some embodiments, the target nucleic acid can be cross-linked to proteins. In some embodiments, the target nucleic acid can be cell-free nucleic acid. Exemplary cell-free nucleic acid includes but are not limited to cell-free DNA, cell-free tumor DNA, cell-free RNA, and cell-free tumor RNA.

In some embodiments, target nucleic acid may be obtained from a biological sample or a patient sample. The term “biological sample” or “patient sample” as used herein includes samples such as tissues and bodily fluids. “Bodily fluids” may include, but are not limited to, blood, serum, plasma, saliva, cerebral spinal fluid, pleural fluid, tears, lactal duct fluid, lymph, sputum, urine, amniotic fluid, and semen. A sample may include a bodily fluid that is “acellular.” An “acellular bodily fluid” includes less than about 1% (w/w) whole cellular material. Plasma and serum are examples of acellular bodily fluids. A sample may include a specimen of natural or synthetic origin (i.e., a cellular sample made to be acellular). The term “Plasma” as used herein refers to acellular fluid found in blood. “Plasma” may be obtained from blood by removing whole cellular material from blood by methods known in the art (e.g., centrifugation, filtration, and the like).

### DNA Polymerases

Exemplary polymerases are provided in the examples section which follows, e.g., Phusion polymerase and Taq DNA ligase (‘Phusion/Taq’) and T4 DNA polymerase and Ampligase (‘T4/Ampligase’). In addition, DNA polymerases can be modified to have reduced reaction rates, reduced or eliminated exonuclease activity, decreased branch fraction, improved complex stability, altered metal cofactor selectivity, and/or other desirable properties as described herein are generally available. DNA polymerases are sometimes classified into six main groups based upon various phylogenetic relationships, e.g., with E. coli Pol I (class A), E. coli Pol II (class B), E. coli Pol III (class C), Euryarchaeotic Pol II (class D), human Pol beta (class X), and E. coli UmuC/DinB and eukaryotic RAD30/xeroderma pigmentosum variant (class Y). For a review of recent nomenclature, see, e.g., Burgers et al. (2001) “Eukaryotic DNA polymerases: proposal for a revised nomenclature” J Biol Chem. 276 (47): 43487-90. For a review of polymerases, see, e.g., Hübscher et al. (2002) “Eukaryotic DNA Polymerases” Annual Review of Biochemistry Vol. 71:133-163; Alba (2001) “Protein Family Review: Replicative DNA Polymerases” Genome Biology 2 (1): reviews 3002.1-3002.4; and Steitz (1999) “DNA polymerases: structural diversity and common mechanisms” J Biol Chem 274:17395-17398. The basic mechanisms of action for many polymerases have been determined. The sequences of literally hundreds of polymerases are publicly available, and the crystal structures for many of these have been determined or can be inferred based upon similarity to solved crystal structures for homologous polymerases. For example, the crystal structure of Φ29 is available.

In addition to wild-type polymerases, chimeric polymerases made from a mosaic of different sources can be used. For example, Φ29-type polymerases made by taking sequences from more than one parental polymerase into account can be used as a starting point for mutation to produce the polymerases of the invention. Chimeras can be produced, e.g., using consideration of similarity regions between the polymerases to define consensus sequences that are used in the chimera, or using gene shuffling technologies in which multiple Φ29-related polymerases are randomly or semi-randomly shuffled via available gene shuffling techniques (e.g., via “family gene shuffling”; see Crameri et al. (1998) “DNA shuffling of a family of genes from diverse species accelerates directed evolution” Nature 391:288-291; Clackson et al. (1991) “Making antibody fragments using phage display libraries” Nature 352:624-628; Gibbs et al. (2001) “Degenerate oligonucleotide gene shuffling (DOGS): a method for enhancing the frequency of recombination with family shuffling” Gene 271:13-20; and Hiraga and Arnold (2003) “General method for sequence-independent site-directed chimeragenesis: J. Mol. Biol. 330:287-296). In these methods, the recombination points can be predetermined such that the gene fragments assemble in the correct order. However, the combinations, e.g., chimeras, can be formed at random. For example, using methods described in Clarkson et al., five gene chimeras, e.g., comprising segments of a Phi29 polymerase, a PZA polymerase, a M2 polymerase, a B103 polymerase, and a GA-1 polymerase, can be generated. Appropriate mutations to improve branching fraction, increase closed complex stability, or alter reaction rate constants or another desirable property can be introduced into the chimeras.

Available DNA polymerase enzymes have also been modified in any of a variety of ways, e.g., to reduce or eliminate exonuclease activities (many native DNA polymerases have a proof-reading exonuclease function that interferes with, e.g., sequencing applications), to simplify production by making protease digested enzyme fragments such as the Klenow fragment recombinant, etc. As noted, polymerases have also been modified to confer improvements in specificity, processivity, and retention time of labeled nucleotides in polymerase-DNA-nucleotide complexes (e.g., WO 2007/076057 by Hanzel et al. and WO 2008/051530 by Rank et al.), to alter branching fraction and translocation, to increase photostability, and to improve surface-immobilized enzyme activities.

Other polymerases that are available, include human DNA Polymerase Beta from R&D systems. DNA polymerase I is available from Epicenter, GE Health Care, Invitrogen, New England Biolabs, Promega, Roche Applied Science, Sigma Aldrich, and many others. The Klenow fragment of DNA Polymerase I is available in both recombinant and protease digested versions, from, e.g., Ambion, Chimerx, eEnzyme LLC, GE Health Care, Invitrogen, New England Biolabs, Promega, Roche Applied Science, Sigma Aldrich and many others. Φ29 DNA polymerase is available from e.g., Epicentre. Poly A polymerase, reverse transcriptase, Sequenase, SP6 DNA polymerase, T4 DNA polymerase, T7 DNA polymerase, and a variety of thermostable DNA polymerases (Taq, hot start, titanium Taq, etc.) are available from a variety of these and other sources. Recent commercial DNA polymerases include Phusion™0 High-Fidelity DNA Polymerase, available from New England Biolabs; GoTaq® Flexi DNA Polymerase, available from Promega; RepliPHI™ Φ29 DNA Polymerase, available from Epicentre Biotechnologies; PfuUltra™ Hotstart DNA Polymerase, available from Stratagene; KOD HiFi DNA Polymerase, available from Novagen; and many others. Biocompare (dot) com provides comparisons of many different commercially available polymerases.

DNA polymerases that are substrates for mutation to reduce reaction rates, reduce or eliminate exonuclease activity, decrease branching fraction, improve closed complex stability, alter metal cofactor selectivity, and/or alter one or more other property described herein include Taq polymerases, exonuclease deficient Taq polymerases, E. coli DNA Polymerase 1, Klenow fragment, reverse transcriptases, Φ29 related polymerases including wild type Φ29 polymerase and derivatives of such polymerases such as exonuclease deficient forms, T7 DNA polymerase, T5 DNA polymerase, RB69 polymerase, etc. Examples of other Φ29-type DNA polymerases, such as B103, GA-1, PZA, Φ15, BS32, M2Y (also known as M2), Nf, G1, Cp-1, PRD1, PZE, SF5, Cp-5, Cp-7, PR4, PR5, PR722, L17, AV-1, Φ21, or the like. For nomenclature, see also, Meijer et al. (2001) “Φ29 Family of Phages” Microbiology and Molecular Biology Reviews, 65(2): 261-287.

Examples are provided below to facilitate a more complete understanding of the disclosure. The following examples illustrate the exemplary modes of making and practicing the disclosure. However, the scope of the disclosure is not limited to specific embodiments disclosed in these Examples, which are for purposes of illustration only, since alternative methods can be utilized to obtain similar results.

### EXAMPLES

### Example 1: Development of SMRT-Tag and SAMOSA-Tag.

Reasoning that the high efficiency of tagmentation and consolidation of protocol steps would similarly facilitate low-input SMS, transposition of hairpin adaptors was optimized to yield long circular molecules for PacBio sequencing²⁴. This principle was then applied to develop two PCR-free multimodal methods: (i) single-molecule real time sequencing by tagmentation (SMRT-Tag) for assaying the genome and epigenome, and (ii) SAMOSA-Tag, which adds a concurrent channel for mapping chromatin structure. SMRT-Tag accurately detected genetic and epigenetic variants from as little as 40 ng of DNA. SAMOSA-Tag maps of single-fiber CTCF and nucleosome occupancy and CpG methylation uncovered metastasis-associated global chromatin deregulation in technically challenging patient-derived prostate cancer xenografts. These results extend tagmentation to PacBio library preparation and have the potential to enable sensitive, scalable, and cellularly resolved single-molecule genomics.

### Results

### Tn5 Transposition Produces PacBio-Compatible Molecules

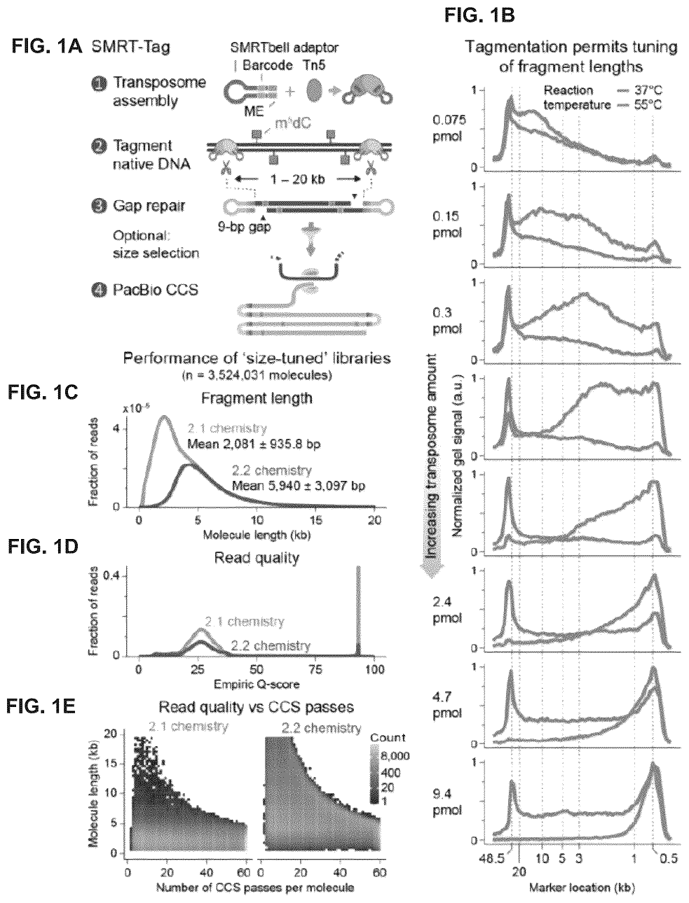

Two technical factors need to be addressed to efficiently generate long (>1 kb) molecules for PacBio SMS via transposition of hairpin adapters into genomic DNA (gDNA; illustrated with the SMRT-Tag workflow, FIG. 1A). First, the conventional Tn5 enzyme used in many short-read sequencing methods optimally produces 100-500 bp fragments. Therefore, a triple-mutant Tn5 enzyme (hereafter referred to as Tn5) was selected, which permitted concentration-dependent control of fragment size²⁵. Tn5 was loaded with custom oligonucleotides comprised of the hairpin PacBio adaptor and mosaic end sequences needed to assemble transposomes. Analytical electrophoresis of gDNA tagmented with adapter-loaded Tn5 at varying reaction conditions confirmed generation of fragments >1-kb long, which are favored at low transposome concentrations and temperature (FIG. 1B). Additional considerations for controlling library size are detailed below.

Second, Tn5 transposition introduces 9-nt gaps into template molecules²⁶ (FIG. 1A), which was sealed for productive SMS. While hairpin transposition has been reported for short-read single-cell genomics¹⁸ and Tn5 is used in some ONT protocols, efficient gap repair to create closed, circular molecules has, to our knowledge, not been reported. Sixty two conditions were tested (Table 1) to optimize gap filling. Two enzyme combinations proved to be the most robust based on yield (FIG. 6) and electrophoretic fragment lengths (FIG. 7) of gDNA subjected to tagmentation, repair, and exonuclease (exo) digestion to select for closed circles: Phusion polymerase and Taq DNA ligase (‘Phusion/Taq’) and T4 DNA polymerase and Ampligase (‘T4/Ampligase’). These produced exo-resistant libraries from as little as 50 ng gDNA, with typical yields >20% of input mass. In all subsequent experiments, Phusion/Taq was used because it provided significantly higher yields on gDNA than T4/Ampligase (p=0.0093, two-sided t-test).

TABLE 1
Gap repair conditions tested in optimizing SMRT-Tag.

| | | Repair condition - |
|---|---|---|
| ID | Repair condition - description | abbreviated name |
| 1 | NEB T4 DNA Polymerase (3 U), Ampligase (10 U), | NEBT4/1x/Amp/2x/ |
| | Ampligase Buffer, 0.1 mM dNTPs, 30 min @ 37° C. | AmpBuf/0.1dNTP |
| 2 | NEB T4 DNA Polymerase (3 U), Ampligase (10 U), | NEBT4/1x/Amp/2x/ |
| | Ampligase Buffer, 1 mM dNTPs, 30 min @ 37° C. | AmpBuf/1dNTP |
| 3 | NEB T4 DNA Polymerase (3 U), Ampligase (10 U), | NEBT4/1x/Amp/2x/ |
| | Ampligase Buffer, 10 mM dNTPs, 30 min @ 37° C. | AmpBuf/10dNTP |
| 4 | NEB T4 DNA Polymerase (3 U), Ampligase (10 U), | NEBT4/1x/Amp/2x/ |
| | Ampligase Buffer, 0.5 mM dNTPs, 30 min @ 37° C. | AmpBuf/0.5dNTP |
| 5 | NEB T4 DNA Polymerase (6 U), Ampligase (10 U), | NEBT4/2x/Amp/2x/ |
| | Ampligase Buffer, 10 mM dNTPs, 30 min @ 37° C. | AmpBuf/10dNTP |
| 6 | NEB T4 DNA Polymerase (3 U), Ampligase (5 U), | NEBT4/1x/Amp/1x/ |
| | Thermo T4 DNA Polymerase Buffer, 1 mM dNTPs, | T4Buf/1dNTP |
| | 0.5 mM NAD+, 30 min @ 37° C. | |
| 7 | NEB T4 DNA Polymerase (3 U), Ampligase (10 U), | NEBT4/1x/Amp/2x/ |
| | Thermo T4 DNA Polymerase Buffer, 0.1 mM | T4Buf/0.1dNTP |
| | dNTPs, 0.5 mM NAD+, 30 min @ 37° C. | |
| 8 | NEB T4 DNA Polymerase (3 U), Ampligase (10 U), | NEBT4/1x/Amp/2x/ |
| | Thermo T4 DNA Polymerase Buffer, 0.5 mM | T4Buf/0.5dNTP |
| | dNTPs, 0.5 mM NAD+, 30 min @ 37° C. | |
| 9 | NEB T4 DNA Polymerase (3 U), Ampligase (10 U), | NEBT4/1x/Amp/2x/ |
| | Thermo T4 DNA Polymerase Buffer, 1 mM dNTPs, | T4Buf/1dNTP |
| | 0.5 mM NAD+, 30 min @ 37° C. | |
| 10 | NEB T4 DNA Polymerase (3 U), Ampligase (10 U), | NEBT4/1x/Amp/2x/ |
| | Thermo T4 DNA Polymerase Buffer, 10 mM | T4Buf/10dNTP |
| | dNTPs, 0.5 mM NAD+, 30 min @ 37° C. | |
| 11 | NEB T4 DNA Polymerase (7.5 U), Ampligase (25 U), | NEBT4/2.5x/Amp/ |
| | Thermo T4 DNA Polymerase Buffer, 1 mM dNTPs, | 5x/T4Buf/1dNTP |
| | 0.5 mM NAD+, 30 min @ 37° C. | |
| 12 | Thermo T4 DNA Polymerase (5 U), Ampligase (10 U), | ThermoT4/1x/Amp/ |
| | Thermo T4 DNA Polymerase Buffer, 1 mM dNTPs, | 2x/T4Buf/1dNTP |
| | 30 min @ 37° C. | |
| 13 | Thermo T4 DNA Polymerase (5 U), Ampligase (10 U), | ThermoT4/1x/Amp/ |
| | Thermo T4 DNA Polymerase Buffer, 10 mM dNTPs, | 2x/T4Buf/10dNTP/ |
| | 2.5 mM NAD+, 30 min @ 37° C. | 2.5NAD |
| 14 | Thermo T4 DNA Polymerase (5 U), Ampligase (10 U), | ThermoT4/1x/Amp/ |
| | Thermo T4 DNA Polymerase Buffer, 0.1 mM dNTPs, | 2x/T4Buf/0.1dNTP/ |
| | 0.5 mM NAD+, 30 min @ 37° C. | 0.5NAD |
| 15 | Thermo T4 DNA Polymerase (5 U), Ampligase (10 U), | ThermoT4/1x/Amp/ |
| | Thermo T4 DNA Polymerase Buffer, 0.5 mM dNTPs, | 2x/T4Buf/0.5dNTP/ |
| | 0.5 mM NAD+, 30 min @ 37° C. | 0.5NAD |
| 16 | Thermo T4 DNA Polymerase (5 U), Ampligase (10 U), | ThermoT4/1x/Amp/ |
| | Thermo T4 DNA Polymerase Buffer, 1 mM dNTPs, | 2x/T4Buf/1dNTP/ |
| | 0.5 mM NAD+, 30 min @ 37° C. | 0.5NAD/30 min |
| 17 | Thermo T4 DNA Polymerase (5 U), Ampligase (10 U), | ThermoT4/1x/Amp/ |
| | Thermo T4 DNA Polymerase Buffer, 10 mM dNTPs, | 2x/T4Buf/10dNTP/ |
| | 0.5 mM NAD+, 30 min @ 37° C. | 0.5NAD |
| 18 | Thermo T4 DNA Polymerase (5 U), Ampligase (10 U), | ThermoT4/1x/Amp/ |
| | Thermo T4 DNA Polymerase Buffer, 1 mM dNTPs, | 2x/T4Buf/1dNTP/ |
| | 0.5 mM NAD+, 60 min @ 37° C. | 0.5NAD/60 min |
| 19 | Thermo T4 DNA Polymerase (5 U), Ampligase (5 U), | ThermoT4/1x/Amp/ |
| | Thermo T4 DNA Polymerase Buffer, 1 mM dNTPs, | 1x/T4Buf/1dNTP/ |
| | 0.5 mM NAD+, 30 min @ 37° C. | 0.5NAD |
| 20 | Thermo T4 DNA Polymerase (5 U), Ampligase (10 U), | ThermoT4/1x/Amp/ |
| | Thermo T4 DNA Polymerase Buffer, 1 mM dNTPs, | 2x/T4Buf/1dNTP/ |
| | 0.5 mM NAD+, 5% PEG4000, 30 min @ 37° C. | 0.5NAD/PEG |
| 21 | Thermo T4 DNA Polymerase (5 U), Ampligase (20 U), | ThermoT4/1x/Amp/ |
| | Thermo T4 DNA Polymerase Buffer, 1 mM dNTPs, | 4x/T4Buf/1dNTP/ |
| | 0.5 mM NAD+, 30 min @ 37° C. | 0.5NAD |
| 22 | Thermo T4 DNA Polymerase (5 U), Ampligase (10 U), | ThermoT4/1x/Amp/ |
| | Thermo T4 DNA Polymerase Buffer, 1 mM dNTPs, | 2x/T4Buf/1dNTP/ |
| | 0.5 mM NAD+, 100 ug/uL BSA, 30 min @ 37° C. | 0.5NAD/BSA |
| 23 | Thermo T4 DNA Polymerase (5 U), Ampligase (10 U), | ThermoT4/1x/Amp/ |
| | NEB CutSmart Buffer, 1 mM dNTPs, 0.5 mM NAD+, | 2x/CutSmartBuf/ |
| | 30 min @ 37° C. | 1dNTP/0.5NAD |
| 24 | Thermo T4 DNA Polymerase (5 U), Ampligase (10 U), | ThermoT4/1x/Amp/ |
| | NEB Buffer2, 1 mM dNTPs, 0.5 mM NAD+, 30 | 2x/NEBuf2/1dNTP/ |
| | min @ 37° C. | 0.5NAD |
| 25 | Thermo T4 DNA Polymerase (10 U), Ampligase (20 U), | ThermoT4/2x/Amp/ |
| | Thermo T4 DNA Polymerase Buffer, 1 mM dNTPs, | 4x/T4Buf/1dNTP/ |
| | 0.5 mM NAD+, 30 min @ 37° C. | 0.5NAD |
| 26 | Thermo T4 DNA Polymerase (10 U), Ampligase | ThermoT4/2x/Amp/ |
| | (20 U), Thermo T4 DNA Polymerase Buffer, 1 mM | 4x/T4Buf/1dNTP/ |
| | dNTPs, 2.5 mM NAD+, 30 min @ 37° C. | 2.5NAD |
| 27 | Thermo T4 DNA Polymerase (12.5 U), Ampligase | ThermoT4/2.5x/ |
| | (25 U), Thermo T4 DNA Polymerase Buffer, 1 mM | Amp/5x/T4Buf/ |
| | dNTPs, 0.5 mM NAD+, 30 min @ 37° C. | 1dNTP/0.5NAD |
| 28 | Thermo T4 DNA Polymerase (5 U), NEB Taq DNA | ThermoT4/1x/Taq/ |
| | Ligase (80 U), NEB Taq DNA Buffer, 1 mM dNTPs, | TaqBuf/1dNTP |
| | 30 min @ 37° C. | |
| 29 | Thermo T4 DNA Polymerase (5 U), NEB T7 DNA | ThermoT4/1x/T7/ |
| | Ligase (3000 U), NEB StickTogether Ligase Buffer, | StickBuf/1dNTP |
| | 1 mM dNTPs, 30 min @ 37° C. | |
| 30 | Thermo T4 DNA Polymerase (5 U), NEB HiFi Taq | ThermoT4/1x/ |
| | DNA Ligase (1 U), NEB HiFi Taq DNA Ligase Buffer, | HiFiTaq/ |
| | 1 mM dNTPs, 30 min @ 37° C. | HiFiTaqBuf/1dNTP |
| 31 | Thermo T4 DNA Polymerase (5 U), NEB 9° N Ligase | ThermoT4/1x/9N/ |
| | (80 U), NEB 9° N Ligase Buffer, 1 mM dNTPs, 30 | 9NBuf/1dNTP |
| | min @ 37° C. | |
| 32 | NEB Phusion High-Fidelity DNA Polymerase (0.8 U), | Phu/1x/Amp/1x/ |
| | Ampligase (2 U), Ampligase Buffer, 0.05 mM dNTPs, | AmpBuf/0.05dNTP/ |
| | 50 mM KCl, 20% DMF, 30 min @ 37° C. | 50KCl/20DMF/30 min |
| 33 | NEB Phusion High-Fidelity DNA Polymerase (0.8 U), | Phu/1x/Amp/1x/ |
| | Ampligase (2 U), Ampligase Buffer, 0.05 mM dNTPs, | AmpBuf/0.05dNTP/ |
| | 50 mM KCl, 10% DMF, 30 min @ 37° C. | 50KCl/10DMF/30 min |
| 34 | NEB Phusion High-Fidelity DNA Polymerase (0.8 U), | Phu/1x/Amp/1x/ |
| | Ampligase (2 U), Ampligase Buffer, 0.05 mM dNTPs, | AmpBuf/0.05dNTP/ |
| | 50 mM KCl, 10% DMF, 30 min @ 37° C. + 15 min @ | 50KCl/10DMF/ |
| | 45° C. | 45 min |
| 35 | NEB Phusion High-Fidelity DNA Polymerase (0.8 U), | Phu/1x/Amp/1x/ |
| | Ampligase (2 U), Ampligase Buffer, 0.8 mM dNTPs, | AmpBuf/0.08dNTP/ |
| | 25 mM KCl, 10% DMF, 60 min @ 37° C. | 25KCl/10DMF/60 min |
| 36 | NEB Phusion High-Fidelity DNA Polymerase (4 U), | Phu/5x/Amp/5x/ |
| | Ampligase (10 U), Ampligase Buffer, 0.05 mM | AmpBuf/0.05dNTP/ |
| | dNTPs, 50 mM KCl, 20% DMF, 30 min @ 37° C. | 50KCl/20DMF/30 min |
| 37 | NEB Phusion High-Fidelity DNA Polymerase (4 U), | Phu/5x/Amp/5x/ |
| | Ampligase (10 U), Ampligase Buffer, 0.05 mM dNTPs, | AmpBuf/0.05dNTP/ |
| | 50 mM KCl, 10% DMF, 30 min @ 37° C. | 50KCl/10DMF/30 min |
| 38 | NEB Phusion High-Fidelity DNA Polymerase (4 U), | Phu/5x/Amp/5x/ |
| | Ampligase (10 U), Ampligase Buffer, 0.05 mM dNTPs, | AmpBuf/0.05dNTP/ |
| | 50 mM KCl, 10% DMF, 30 min @ 37° C. + 15 min @ | 50KCl/10DMF/ |
| | 45° C. | 45 min |
| 39 | NEB Phusion High-Fidelity DNA Polymerase (4 U), | Phu/5x/Amp/5x/ |
| | Ampligase (10 U), Ampligase Buffer, 0.8 mM dNTPs, | AmpBuf/0.8dNTP/ |
| | 25 mM KCl, 10% DMF, 60 min @ 37° C. | 25KCl/10DMF/60 min |
| 40 | NEB Phusion High-Fidelity DNA Polymerase (4 U), | Phu/5x/Amp/5x/ |
| | Ampligase (10 U), Ampligase Buffer, 0.8 mM dNTPs, | AmpBuf/0.8dNTP/ |
| | 25 mM KCl, 60 min @ 37° C. | 25KCl/60 min |
| 41 | NEB Phusion High-Fidelity DNA Polymerase (0.32 U), | Phu/0.4x/Taq/ |
| | NEB Taq DNA Ligase (80 U), NEB Taq DNA | TaqBuf/0.8dNTP |
| | Ligase Buffer, 0.8 mM dMTPs, 30 min @ 37° C. | |
| 42 | NEB Phusion High-Fidelity DNA Polymerase (0.32 U), | Phu/0.4x/Taq/ |
| | NEB Taq DNA Ligase (80 U), NEB Taq DNA Ligase | TaqBuf/0.8dNTP/ |
| | Buffer, 0.8 mM dMTPs, 10% DMF, 30 min @ 37° C. | 10DMF |
| 43 | NEB Phusion High-Fidelity DNA Polymerase (0.8 U), | Phu/1x/Taq/ |
| | NEB Taq DNA Ligase (80 U), Ampligase Buffer, 0.05 | AmpBuf/0.05dNTP/ |
| | mM dMTPs, 50 mM KCl, 10% DMF, 30 min @ 37° C. | 50KCl/10DMF |
| 44 | NEB Phusion High-Fidelity DNA Polymerase (2 U), | Phu/2.5x/Taq/ |
| | NEB Taq DNA Ligase (80 U), NEB Taq DNA Ligase | TaqBuf/0.8dNTP/ |
| | Buffer, 0.8 mM dMTPs, 30 min @ 37° C. | 30 min |
| 45 | NEB Phusion High-Fidelity DNA Polymerase (2 U), | Phu/2.5x/Taq/ |
| | NEB Taq DNA Ligase (80 U), NEB Taq DNA Ligase | TaqBuf/0.8dNTP/ |
| | Buffer, 0.8 mM dMTPs, 60 min @ 37° C. | 60 min |
| 46 | NEB Phusion High-Fidelity DNA Polymerase (4 U), | Phu/5x/Taq/TaqBuf/ |
| | NEB Taq DNA Ligase (80 U), NEB Taq DNA Ligase | 0.8dNTP/60 min |
| | Buffer, 0.8 mM dMTPs, 60 min @ 37° C. | |
| 47 | NEB PreCR Repair Mix (1 U), ThermoPol Reaction | PreCR/ |
| | Buffer, 0.1 mM dNTPs, 0.5 mM NAD+, 30 min @ | ThermoPolBuf/ |
| | 37° C. | 0.1dNTP/0.5NAD |
| 48 | NEB Bst DNA Polymerase, Full Length (0.8 U), NEB | Bst/Taq/ |
| | Taq DNA Ligase (60 U), ThermoPol Reaction Buffer, | ThermoPolBuf/ |
| | 1 mM dNTPs, 0.5 mM NAD+, 30 min @ 37° C. | 1dNTP/0.5NAD |
| 49 | NEB Phusion High-Fidelity DNA Polymerase (2 U), | Phu/9N/9NBuf/ |
| | NEB 9° N Ligase (80 U), NEB 9° N Ligase Buffer, | 0.8dNTP |
| | 0.8 mM dNTPs, 30 min @ 37° C. | |
| 50 | NEB Phusion High-Fidelity DNA Polymerase (2 U), | Phu/HiFiTaq/ |
| | NEB HiFi Taq DNA Ligase (1 U), NEB HiFi Taq | HiFiTaqBuf/ |
| | DNA Ligase Buffer, 0.8 mM dNTPs, 60 min @ 37° C. | 0.8dNTP |
| 51 | NEB Q5 High-Fidelity DNA Polymerase (0.4 U), | Q5/Amp/Q5Buf/ |
| | Ampligase (10 U), NEB Q5 Reaction Buffer, 0.2 | 0.2dNTP/0.5NAD |
| | mM dNTPs, 0.5 mM NAD+, 30 min @ 37° C. | |
| 52 | NEB Phusion High-Fidelity DNA Polymerase (2 U), | Phu/2.5x/Taq/ |
| | NEB Taq DNA Ligase (80 U), NEB Taq DNA Ligase | TaqBuf/0.8dNTP/ |
| | Buffer, 0.8 mM dNTPs, 0.8 mM ATP, T4 PNK (5 U), | PreCRMix |
| | homemade PreCR Repair Mix, 30 min @ 37° C. + | |
| | 60 min @ 37° C. | |
| 53 | Thermo T4 DNA Polymerase (5 U), Ampligase (10 U), | ThermoT4/1x/Amp/ |
| | Thermo T4 DNA Polymerase Buffer, 1 mM dNTPs, | 2x/T4Buf/1dNTP/ |
| | 0.5 mM NAD+, T4 PNK (5 U), 30 min @ 37° C. | 0.5NAD/PNK |
| 54 | NEB T4 DNA Polymerase (3 U), NEB HiFi Taq | NEBT4/1x/HiFiTaq/ |
| | Ligase (1 U), NEB Buffer2, 1 mM dNTPs, 0.8 mM | 1x/NEBuf2/1dNTP/ |
| | ATP, T4 PNK (5 U), 0.5 mM NAD+, homemade | PreCRMix |
| | PreCR Repair Mix, 30 min @ 37° C. + 30 min @ | |
| | 37° C. | |
| 55 | NEB T4 DNA Polymerase (9 U), NEB HiFi Taq | NEBT4/3x/HiFiTaq/ |
| | Ligase (3 U), NEB Buffer2, 1 mM dNTPs, 0.8 mM | 3x/NEBuf2/1dNTP/ |
| | ATP, T4 PNK (5 U), 0.5 mM NAD+, homemade | PreCRMix |
| | PreCR Repair Mix, 30 min @ 37° C. + | |
| | 30 min @ 37° C. | |
| 56 | Thermo T4 DNA Polymerase (5 U), NEB HiFi Taq | ThermoT4/1x/ |
| | Ligase (1 U), Thermo T4 DNA Polymerase Buffer, | HiFiTaq/1x/T4Buf/ |
| | 1 mM dNTPs, 0.8 mM ATP, T4 PNK (5 U), 0.5 mM | 1dNTP/PreCRMix |
| | NAD+, homemade PreCR Repair Mix, 30 min @ | |
| | 37° C. + 30 min @ 37° C. | |
| 57 | Thermo T4 DNA Polymerase (5 U), Ampligase (10 U), | ThermoT4/1x/Amp/ |
| | Thermo T4 DNA Polymerase Buffer, 1 mM dNTPs, | 2x/T4Buf/1dNTP/ |
| | 0.8 mM ATP, T4 PNK (5 U), 0.5 mM NAD+, homemade | PreCRMix |
| | PreCR Repair Mix, 30 min @ 37° C. + 30 min @ 37° C. | |
| 58 | Thermo T4 DNA Polymerase (15 U), Ampligase (30 U), | ThermoT4/3x/Amp/ |
| | Thermo T4 DNA Polymerase Buffer, 1 mM dNTPs, | 6x/T4Buf/1dNTP/ |
| | 0.8 mM ATP, T4 PNK (5 U), 0.5 mM NAD+, | PreCRMix |
| | homemade PreCR Repair Mix, 30 min @ 37° C. + | |
| | 30 min @ 37° C. | |
| 59 | Thermo T4 DNA Polymerase (5 U), Ampligase (10 U), | ThermoT4/1x/Amp/ |
| | NEB Buffer2, 1 mM dNTPs, 0.8 mM ATP, T4 PNK | 2x/NEBuf2/1dNTP/ |
| | (5 U), 0.5 mM NAD+, homemade PreCR Repair Mix, | PreCRMix |
| | 30 min @ 37° C. + 30 min @ 37° C. + 30 min @ 37° C. | |
| 60 | NEB Phusion High-Fidelity DNA Polymerase (2 U), | Phu/2.5x/Taq/ |
| | NEB Taq DNA Ligase (80 U), NEB Taq DNA Ligase | TaqBuf/0.8dNTP/ |
| | Buffer, 0.8 mM dMTPs, 0.8 mM ATP, T4 PNK (5 U), | 1NAD/PreCRMix |
| | 1 mM NAD+, 50 mM KCl, homemade PreCR Repair | |
| | Mix, 30 min @ 37° C. + 30 min @ 37° C. + 30 min @ | |
| | 37° C. | |
| 61 | NEB Phusion High-Fidelity DNA Polymerase (4 U), | Phu/5x/Amp/5x/ |
| | Ampligase (10 U), Ampligase Buffer, 0.8 mM dMTPs, | AmpBuf/0.8dNTP/ |
| | 0.8 mM ATP, T4 PNK (5 U), 0.5 mM NAD+, 50 mM | PreCRMix |
| | KCl, homemade PreCR Repair Mix, 30 min @ 37° C. + | |
| | 30 min @ 37° C. + 30 min @ 37° C. | |

### SMRT-Tag: Tunable and Multiplexable PacBio Library Construction

Direct transposition was applied in SMRT-Tag, a simple method for whole genome analysis, and explored library and sequencing characteristics. To evaluate the sequencing efficiency of SMRT-Tag, 120 ng of HG002 gDNA (equivalent to ˜20,000 human cells) was tagmented in 8 separate reactions and solid-phase reversible immobilization (SPRI) beads were used to fractionate the resulting libraries for sequencing using PacBio's proprietary 2.1 and 2.2 polymerases optimized for short and long templates, respectively. Circular consensus sequencing (CCS) read length distributions of the 3,524,301 molecules (14.3 Gb total) sequenced over two runs were concordant with size selection and polymerase choice (FIG. 1C; 2,081 +935.8 bp vs. 5,940±3,097 bp for polymerases 2.1 and 2.2, respectively; mean±standard deviation [s.d.]). The per-read quality scores (Qscores; FIG. 1D) and number of CCS passes (FIG. 1E) were sufficient for PacBio high-fidelity ('HiFi') sequencing with >99% (>Q20) base accuracy, which typically requires ≥5 redundant passes per molecule.

To assess demultiplexing using the 8-nt barcode included in the SMRT-Tag hairpin adaptor (FIG. 1A), low-pass sequencing was performed of libraries pooled after tagmentation, gap repair, and exo digestion of gDNA from the extensively genotyped HG002, HG003, and HG004 human trio (in total, seven 80-ng reactions sequenced to 0.75X HG002, 1.39X HG003, and 1.30X HG004 depths; FIG. 8A). The “left” and “right” barcodes of molecules were inspected, which were identical (99.9% concordance; FIG. 8B). Taking advantage of the pedigree to query genotype mixing of multiplexed libraries, it was confirmed that HG003 and HG004 (unrelated parents) share few private SNVs (0.60% HG003 vs. HG004; 0.67% HG004 vs. HG003), while HG002 (child) is a mixture of parental genotypes (33.1% overlap; FIG. 8C). Second, to determine if samples can be multiplexed immediately after tagmentation, gDNA libraries were sequenced from four separate reactions pooled before gap repair and exo digestion (FIG. 8D). Barcode concordance (99.9%, FIG. 8E) and Smith-Waterman barcode alignment scores reported by the lima demultiplexer (mean 97.9, s.d. 6.78, normalized scale 0-100; FIG. 8F) were excellent. This confirmed there is no tagging of previously transposed molecules during gap repair, exo cleanup, and pooling and is consistent with the zero-turnover activity of Tn5.

Finally, to illustrate the tunability of SMRT-Tag, gDNA was tagmented at varying Tn5 concentrations and reaction temperatures, and multiplexed libraries for sequencing. The resulting read length distributions confirmed that Tn5: DNA ratio and temperature can be varied to shift library size distributions (FIGS. 9A-9C). The mean and standard deviation of fragment lengths were respectively controllable over nearly 11-and 18-fold dynamic ranges, offering an important reference point for implementing the approach (FIG. 9C).

For all experiments, unless noted, libraries were multiplexed to minimize sequencing cost. It was concluded that SMRT-Tag generates multiplexable PCR-free PacBio libraries from low input DNA amounts for multiplex sequencing. pcl SMRT-Tag Permits Accurate, Low-Input Genetic and Epigenetic Variant Detection

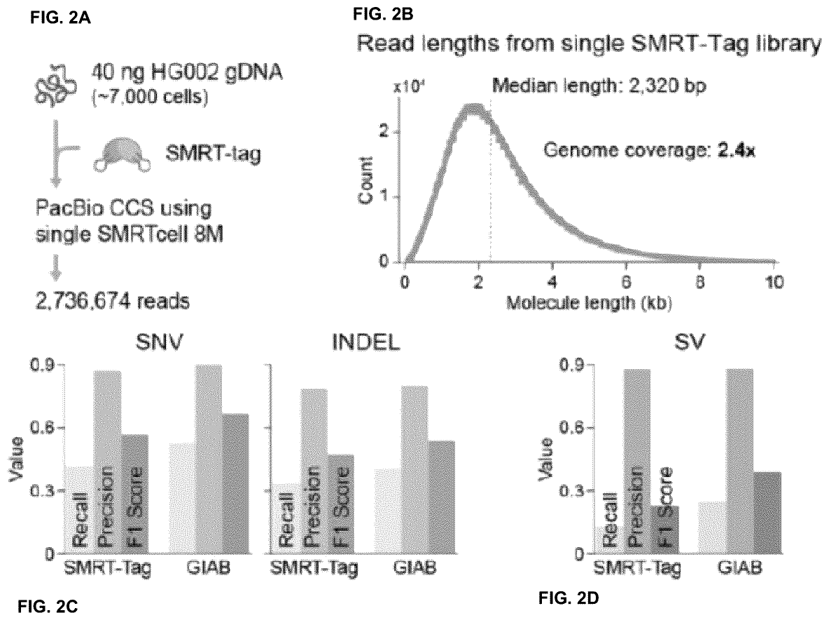

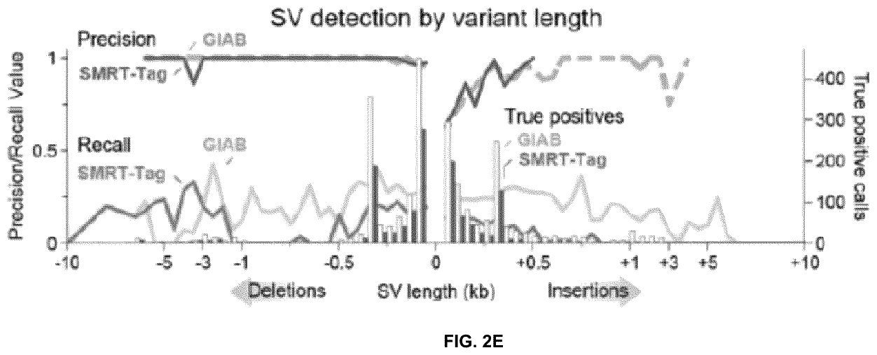

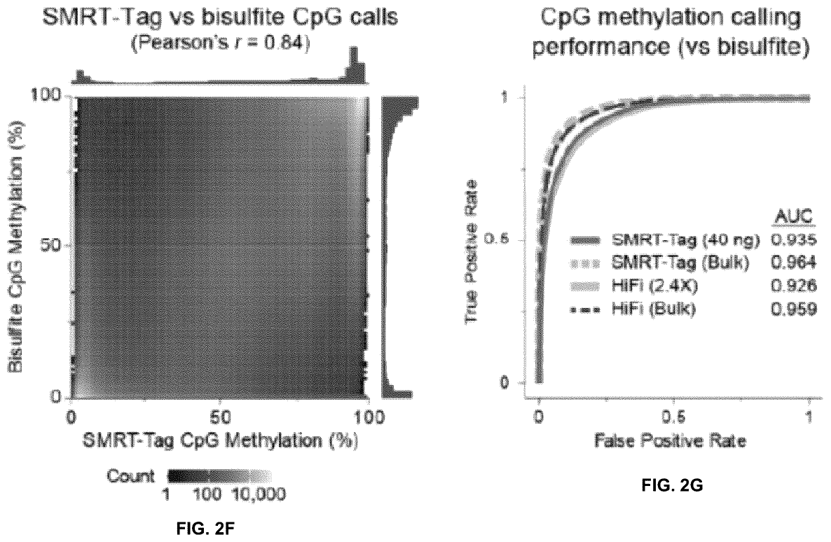

It was next sought to establish the sensitivity and variant-calling accuracy of SMRT-Tag. It was first determined whether libraries can be generated at the minimum on-plate loading concentration (OPLC) for PacBio Sequel II flow cells of 20-40 pM. One SMRT-Tag library generated from 40 ng HG002 gDNA (˜7,000 human cell equivalents) was sequenced achieving 37 PM OPLC (FIG. 2A). A single flow cell yielded 2,736,674 CCS reads with 2.32 kb median length, equivalent to ˜2.43X genome coverage (FIG. 2B). While this depth is suboptimal for routine genotyping applications, it was queried whether the data quality was sufficient for variant detection. Single nucleotide (SNVs) and insertion/deletion (indel) variants were called using Deep Variant and structural variants (SVs) with pbsv from low-input SMRT-Tag and coverage-matched ligation-based libraries sequenced by the Genome in a Bottle (GIAB) consortium²⁷. To evaluate accuracy, variants were benchmarked detected against the gold-standard GIAB high-confidence HG002 callset (FIGS. 2C-2E)²⁸. Comparing SMRT-Tag and ligation-based libraries, similar recall was observed (0.420 vs. 0.527 for SNVs and 0.338 vs. 0.408 for indels), precision (0.870 vs. 0.898 for SNVs and 0.785 vs. 0.797 for indels), and F1 score (0.566 vs. 0.664 for SNVs and 10 0.380 vs. 0.539 for indels; FIG. 2C). Performance for SVs was slightly lower (recall 0.129 vs. 0.25, precision 0.877 vs. 0.879, and F1 score 0.225 vs. 0.389; FIG. 2D) likely due to shorter reads affecting resolution of large indels.

In PacBio SMS, nucleobase modifications are inferred from stereotyped changes in real-time polymerase kinetics during nucleotide addition, offering an opportunity for simultaneous genotyping and epigenotyping²⁹. To assess detection of CpG methylation, positions of m⁵dC were predicted using PacBio's primrose software, which assigns methylation probabilities to CpGs via a convolutional neural network that combines kinetic data from multiple CCS passes. Primrose methylation calls from SMRT-Tag and ligation-based PacBio SMS were compared against gold-standard bisulfite sequencing data³⁰. Per-CpG methylation calls were tightly correlated between SMRT-Tag and bisulfite m5dC datasets (Pearson's r=0.84; FIG. 2E). Framing CpG methylation calling as a classification problem (FIG. 2F), excellent performance was observed, measured by area-under-curve (AUC), with SMRT-Tag and ligation-based datasets demonstrating similar AUC (0.935 vs. 0.926, respectively).

Finally, to compare performance at higher depths, additional HG002 SMRT-Tag libraries were sequenced to 11.2X median coverage (34.24 Gb on 6 Sequel II flow cells). SNV, indel, and SV calls from SMRT-Tag and coverage-matched ligation-based libraries were compared against the GIAB HG002 benchmark. Similar recall was found for (0.970 SMRT-Tag vs. 0.970 ligation-based PacBio for SNVs and 0.911 vs. 0.907 for indels), precision (0.995 vs. 0.995 for SNVs and 0.955 vs. 0.949 for indels), F1 score (0.983 vs. 0.982 for SNVs and 0.932 vs. 0.928 for indels), and AUC (0.969 vs. 0.968 for SNVs and 0.902 vs. 0.897 for indels; FIGS. 10A-10D). CpG methylation detected using high coverage SMRT-Tag was on par with short-read bisulfite (FIG. 10E) and ligation-based PacBio (FIG. 10F) data. SMRT-Tag also resolved variants within segmental duplications, repeats, the MHC locus, and other challenging regions (FIG. 6A; F1 scores 0.977 SMRT-Tag vs. 0.967 ligation-based PacBio for SNVs and 0.912 vs. 0.905 for indels across all regions with differences likely due to sequencing chemistry) and at varying levels of coverage (FIG. 6B). Taken together, these results demonstrate the strong technical concordance between tagmentation and ligation³⁵ based libraries and the sensitive detection of genetic and epigenetic variation by SMRT-Tag.

### Mapping Single-Fiber Chromatin Accessibility and CpG Methylation With SAMOSA-Tag

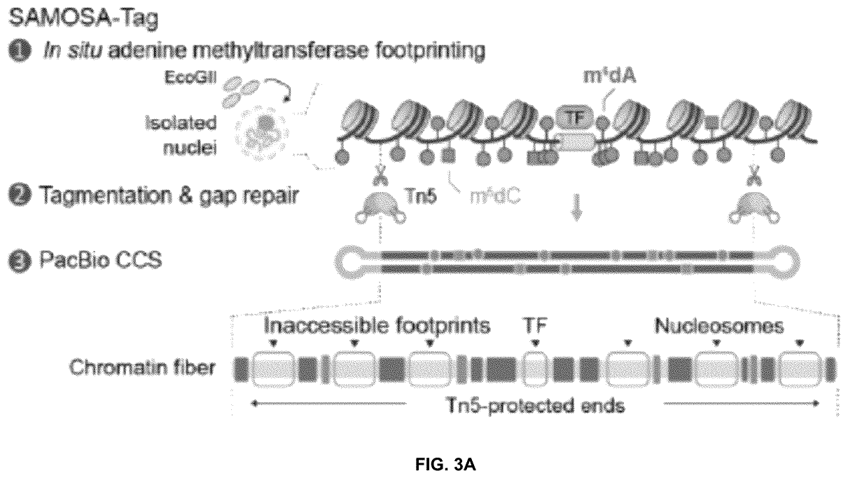

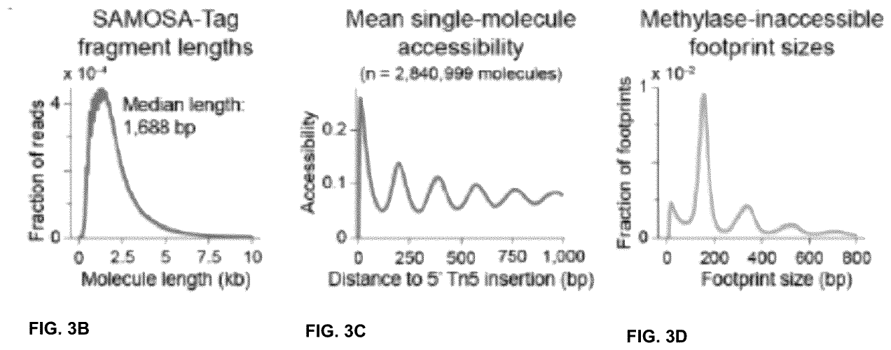

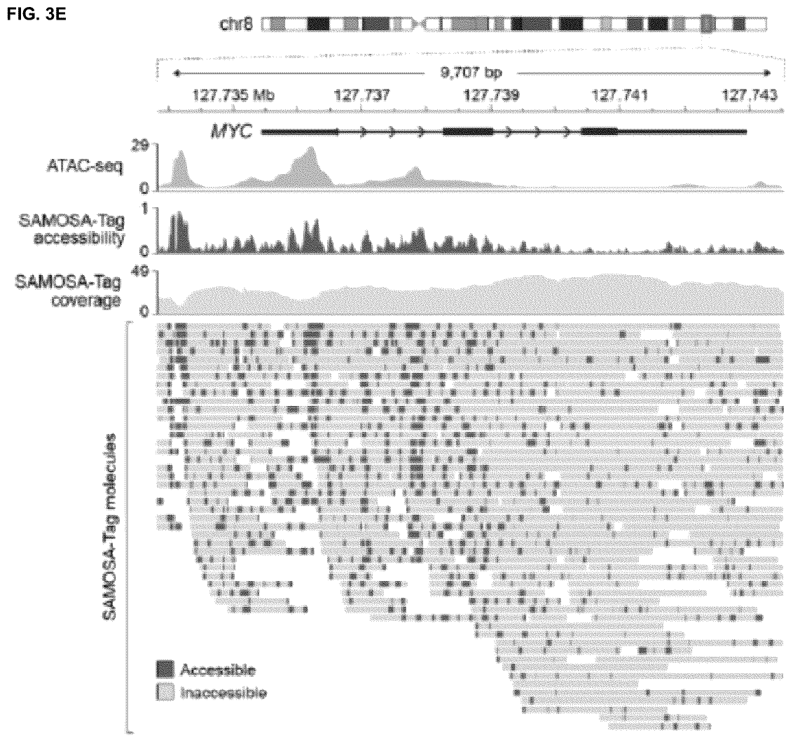

Tagmentation is the basis for ATAC-seq, a popular method for profiling chromatin accessibility¹⁶. Reasoning that Tn5 could be used to lower the microgram-range input needed for single-molecule chromatin accessibility assays developed by the inventors, a tagmentation-assisted single-molecule adenine methylated oligonucleosome sequencing assay (SAMOSA-Tag; FIG. 3A) was optimized. In SAMOSA-Tag, nuclei are methylated in situ with the EcoGII modAase and tagmented using hairpin-loaded Tn5 under conditions optimal for ATAC-seq³¹. DNA is then purified, gap-repaired, and sequenced. As proof-of-concept, SAMOSA-Tag was applied to 50,000 nuclei from MYC-amplified OS152 human osteosarcoma cells³², and used a convolutional neural network-hidden Markov model (CNN-HMM)¹¹ to call inaccessible protein-DNA interaction ‘footprints’ from m⁶dA natively detected by PacBio SMS. In total, 3,640,652 molecules (7.79 Gb) across eight replicates were sequenced. Reflecting transposition of chromatin in nuclei, SAMOSA-Tag CCS read lengths displayed characteristic oligonucleosomal banding (FIG. 3B). When aligned at 5′ ends, molecules had periodic accessibility signal, consistent with transposition adjacent to nucleosomal barriers (FIG. 3C). Individual single-molecule footprint sizes also corresponded to expected mono-, di-, tri-, etc. nucleosomes (FIG. 3D). Finally, single-fiber accessibility visualized in the genomic context, e.g., at the amplified MYC locus (FIG. 3E) and at copy number loss and neutral loci (FIGS. 12, 13A, 13B), correlated well with ATAC-seq. Importantly, there was only a mild enrichment of SAMOSA-Tag insertions for transcription start sites (TSSs; FIG. 14A). However, insertions tended to occur proximal to predicted CCCTC-binding factor (CTCF) binding sites (FIG. 14B), consistent with blocked Tn5 transposition by strong barrier elements. This subtle preference was also reflected in the fraction of insertions falling near TSSs and CTCF sites (FIG. 14C; 1.51-and 1.58-fold enrichment above background, respectively) and is consistent with propensities reported for Tn5-based shotgun Illumina libraries³³. Finally, SAMOSA-Tag generalized well to mouse embryonic stem cells (mESCs; FIGS. 15A-15C), recovering characteristic ‘footprints’ around predicted Ctcf and Rest binding sites, which clustered into distinct accessibility patterns (FIGS. 15D, 15E). SAMOSA-Tag can also be performed ex situ wherein DNA is extracted from footprinted nuclei before tagmentation. The barrier effect apparent upon aligning 5′ read ends is abrogated in ex situ SAMOSA-Tag (FIG. 15B), highlighting the flexibility of the approach for applications requiring more coverage uniformity.

### Integrative Measurement of CpG Methylation and Single-Fiber Chromatin Accessibility

The separability of PacBio polymerase kinetics into modA and m5dC channels affords the opportunity to concurrently ascertain DNA sequence, CpG methylation, and single-fiber chromatin accessibility to exogenous adenine methyltransferases in a single assay. m⁶dA accessibility and CpG methylation was first examined at CTCF sites predicted from ChIP-seq in the U2OS osteosarcoma cell line³⁴. Hallmarks of CTCF binding were recovered including flanking positioned nucleosomes, decreased accessibility immediately at the motif (compatible with exclusion of EcoGII by bound CTCF), and depressed CpG methylation within motifs (FIG. 4A). Taking advantage of the single-molecule resolution of SAMOSA-Tag, the differing fiber structures that contribute to the ensemble average chromatin and methylation profiles (FIG. 4A) were deconvolved using Leiden clustering³⁵ (example of 4 clusters shown in FIG. 4B; cluster sizes in FIG. 16). Analysis of pattern-specific average m⁵dC signal (FIG. 4C) revealed the lowest CpG methylation at CTCF-bound (cluster 1) and unbound/accessible (cluster 2) motif fiber patterns, consistent with prior results³⁶. Two additional analyses confirmed minimal confounding of m⁵dC and m⁶dA signals: First, primrose CpG score distributions of EcoGII untreated negative control and footprinted SAMOSA-Tag libraries were concordant (FIG. 17A). Second, average CpG methylation surrounding predicted CTCF sites on fibers with inaccessible motifs compared to those with footprinted motifs was tightly correlated (FIG. 17B).

The inventors previously demonstrated that single-fiber chromatin accessibility data can be used to segment the genome by regularity and average spacing of nucleosomes (nucleosome-repeat length, NRL) 4,37. These studies relied on complementary epigenomic assays to ascertain the distribution of ‘fiber types’ (i.e., clusters of molecules with unique regularity or NRL) in euchromatic and heterochromatic domains. It was sought to improve on these analyses by directly assessing fiber structure variation with jointly resolved single-molecule CpG content and methylation. To do so, SAMOSA-Tag molecules were grouped into four bins (FIG. 4D) gated on CpG density (>10 CpG dinucleotides/kb) and primrose score (average score >0.5). Fiber types were then defined by clustering m⁶dA accessibility autocorrelation for each molecule ≥1 kb in length^{4,37}. After removing artifactual molecules, 7 distinct clusters were obtained (FIG. 4E; cluster sizes in FIG. 18) effectively stratifying the OS152 genome by NRL (clusters NRL178-NRL208) and regularity (cluster IR, irregular spacing). Finally, a series of enrichment tests were carried out to assess domain-specific fiber composition across the four CpG content and methylation bins (FIG. 4F; reproducibility shown in FIG. 19). Two findings relevant to chromatin regulation are highlighted: first, putative hypomethylated CpG islands (high CpG content, low CpG methylation) are enriched for fibers that are irregular (odds ratio [O.R.] for cluster IR=1.42, p˜0) or have long NRLs (NRL208 O.R.=1.09, p=4.43×10-64; NRL197 O.R.=1.11, p=1.49×10-58); and second, likely hypermethylated, CpG rich repeats (high CpG content, high CpG methylation) are enriched for fibers that are irregular (IR O.R.=1.14, p=1.3×10-139) or have short NRLs (NRL172 O.R.=1.24; p˜0). These results are consistent with the in vivo observations of active promoters and heterochromatin in human cells⁴ and mESCs³⁷, pointing to a conserved fiber chromatin structure within these domains. Together, these analyses show that SAMOSA-Tag generates multimodal, genome-wide single-molecule chromatin accessibility data from tens of thousands of cells.

### SAMOSA-Tag of Patient-Derived Prostate Cancer Xenografts

One area where SAMOSA-Tag could have immediate utility is in the study of disease models such as patient derived cancer xenografts (PDXs) where samples are limited. There are two key challenges with PCR-free PacBio profiling of PDXs propagated in mice: first, following tumor engraftment and growth, cancer cells must be enriched and separated from mouse cells by fluorescence-activated cell sorting (FACS); second, cells and nuclei from metabolically active or necrotic tumors are often fragile and have damaged native DNA, which impedes sequencing. It was thus sought to apply SAMOSA-Tag to generate the first single-fiber chromatin accessibility data from PDX models. PDXs were generated from matched primary and metastatic tumors resected from a patient with castration-resistant prostate cancer³⁸, and ˜180,000 nuclei were isolated and footprinted from one mouse each per model (FIG. 5A; FACS gates shown in FIGS. 20A-20D). To account for the technical difficulty of working with precious PDX samples while ensuring reproducibility, we opted conservatively to perform six replicate SAMOSA-Tag reactions (˜30,000 nuclei/reaction). Primary and metastatic PDX libraries were sequenced to depths of 0.32×(0.95 Gb [22.8%] human alignment) and 0.53×(1.57 Gb [95.9%] human alignment). PDX SAMOSA-Tag had similar technical characteristics to mESC and OS152 experiments (FIGS. 21A-21B). Future optimization of cell enrichment, DNA damage repair, and nuclei purification will likely permit higher per sample coverage using lower input than in the proof-of-concept presented here.

Altered CTCF expression and occupancy have been tied to hyperactive androgen signaling³⁹ and prostate cancer progression⁴⁰. To examine single-molecule chromatin accessibility and CTCF binding in primary and metastatic tumor cells (FIG. 22A), we clustered PDX SAMOSA-Tag reads aligned to CTCF sites predicted using ENCODE ChIP-seq in LnCaP prostate cancer cells. This revealed multiple clusters (FIG. 22B) reflecting varying nucleosome occupancy patterns around the CTCF motif (patterns NO1-NO5), direct CTCF occupancy (pattern A), and ‘hyper-accessible’ fibers devoid of nucleosomes flanking the motif (pattern HA) similar to OS152 and mESC SAMOSA-Tag (FIG. 4E, FIG. 15A). Visualizing differential fiber type usage (FIG. 22C) suggested intriguing metastasis-specific shifts in cluster usage, including a decrease in the stereotypic nucleosome phasing at CTCF bound sites (pattern A) in favor of pattern HA. Analysis of concurrently measured m⁵dC within these clusters suggested subtle preliminary differences in CpG methylation correlated with single-fiber CTCF motif occupancy patterns (FIG. 22D).

Finally, it was queried whether single-fiber chromatin architecture differs between matched primary and metastatic tumors (FIG. 23A). Unsupervised Leiden clustering of autocorrelated single-molecule m⁶dA signal from primary and metastatic PDXs yielded six fiber types (FIG. 5B): four regular clusters with NRLs ranging 171 to 208 bp and two irregular clusters (IR1 and IR2). Using published annotations for healthy human prostate as a reference⁴¹, the relative enrichment of fiber types across epigenomic domains was determined (FIG. 23B). Applying a logistic regression framework to nominate significant differences in domain-specific fiber usage, several patterns of interest were identified for follow up in future studies (FIG. 5C). For instance, metastatic tumor cells were significantly enriched for irregular fibers (IR1 and IR2) in heterochromatic domains such as KRAB zinc-finger genes (ZNF/Rpts; IR1 log2 fold-change [Δ]=0.77, q=7.56×10-7; IR2 Δ=1.03, q=6.15×10-15) and regions harboring marks of constitutive heterochromatin (Het; IR1 Δ=1.22, q 1.45×10-177; IR2 Δ=1.25; q=4.46×10-125). In contrast, distal enhancers were significantly depleted for fibers with specific NRLs (e.g., active enhancer 1 [EnhA1]; NRL182 Δ=−1.11, q=1.07×10-71). These data hint at involvement of ATP-dependent chromatin remodelers such as the Brahma-associated factor (BAF) complex in metastasis-associated nucleosome eviction and chromatin disorganization (FIG. 5D). While BAF has already been implicated as a driver of prostate cancer progression⁴², mechanistic studies are needed to evaluate the proposed preliminary model. Taken together, these data demonstrate the potential of SAMOSA-Tag to yield biological insights in challenging disease models.

### Discussion

Direct Tn5 transposition of hairpin adaptors was optimized as a general strategy for preparing amplification-free, multiplexable PacBio libraries from limiting amounts of native input DNA. This principle was applied to develop two methods that take advantage of the simultaneous readout of modified and unmodified bases by SMS and highlight the broad potential of Tn5-based PacBio library preparation. First, tagmentation coupled with PacBio HiFi sequencing (SMRT-Tag) allowed detection of genetic variation and CpG methylation from as little as 40 ng gDNA (˜7,000 human cells) with accuracy comparable to conventional whole genome and bisulfite sequencing. Second, tagmentation of as few as 30,000-50,000 nuclei following adenine methyltransferase chromatin footprinting (SAMOSA-Tag) permitted concurrent single-fiber DNA sequence, CpG methylation, and chromatin accessibility profiling in one assay. Using SAMOSA-Tag libraries multiplexed to maximize sequencing yield, CTCF binding, nucleosome architecture, and CpG methylation in osteosarcoma cells was resolved. The first single-molecule epigenome analyses in a preclinical disease model was also carried out, uncovering global chromatin dysregulation associated with metastatic progression in technically challenging prostate cancer PDX cells.

It is anticipated that tagmentation-based protocols will address several obstacles to single-molecule genomics. Simplification of library preparation by combining DNA fragmentation and adapter ligation steps and the high efficiency of Tn5 transposition permitted 90-99% input reduction for SMRT-Tag and SAMOSA-Tag, placing monoplex sequencing at the lower limit of the PacBio platform within reach. The ability to profile unamplified DNA has implications for basic and translational analyses of rare cell populations that integrate the breadth of nucleotide, structural, and epigenomic variation natively captured by SMS without chemical conversion. Importantly, in situ tagmentation also obviates the need for DNA purification, raising the exciting prospect of multimodal genomics with both single-cell and single-molecule resolution. It is envisioned that future developments including droplet-or combinatorial barcoding-based cellular indexing^{21,23,43} will extend massively parallel PCR-free single-molecule assays to individual cells, enabling applications ranging from strand²⁵ specific somatic variant detection⁴⁴, to haplotype-resolved de novo assembly, and cell type classification.

It was demonstrated herein that flow cells can be efficiently loaded with as little as 40 ng starting input mass. The length of molecules is primarily controlled by transposome concentration and optional bead-based size selection. The limited input amount precludes gel-based size fractionation. Further, the inverse proportionality between length and molarity for a given input amount implies that more starting material or pooling at higher plexity would be needed to take advantage of 15-20 kb PacBio reads and yield deep coverage. This is salient for, e.g., structural variant discovery, as breakpoint-spanning long molecules are less abundant in SMRT-Tag than ligation based libraries. While these have been partially addressed this by demonstrating tunability of tagmentation, adapting engineered²⁵ and bead-linked⁴⁵ transposases may offer finer control of molecule length in the future. In the experiments herein, high-quality data from pooled replicates of 30,000-50,000 nuclei each was generated. Optimizations including mild fixation, miniaturized methylation reactions, or immobilization of nuclei on beads⁴⁶ could further relax this constraint. More generally, SMRT-Tag and SAMOSA-Tag add to a growing series of technological innovations centered around third-generation sequencing, including Cas9-targeted sequence capture⁴⁷, combinatorial-indexing-based plasmid reconstruction⁴⁸, and concatenation-based isoform-resolved transcriptomics49 The widespread adoption of short-read genomics in basic and clinical applications, and the transition from bulk to single-cell assays was catalyzed by tools that simplified library preparation and reduced input requirement. Direct transposition offers similar promise for rapidly maturing third-generation sequencing technologies in enabling scalable, sensitive, and high-fidelity telomere-to-telomere genomics and epigenomics.

TABLE 2
Gap-repair condition efficiencies evaluated in optimizing SMRT-Tag.

| | | | | | Repair | Subgroup | Subgroup |
|---|---|---|---|---|---|---|---|
| | | | Reaction | | condition - | mean | std. dev. |
| Repair | | Efficiency | Input | | abbreviated | repair | repair |
| condition | ID | (%) | Mass | Source | name | efficiency | efficiency |
| Phu/Amp | 34 | 56.03 | 160 | Promega | Phu/1x/Amp/1x/ | 36.48 | 27.6478751 |
| | | | | | AmpBuf/0.05dNTP/ | | |
| | | | | | 50KCl/10DMF/ | | |
| | | | | | 45 min | | |
| Phu/Amp | 34 | 16.93 | 160 | Promega | Phu/1x/Amp/1x/ | | |
| | | | | | AmpBuf/0.05dNTP/ | | |
| | | | | | 50KCl/10DMF/ | | |
| | | | | | 45 min | | |
| Phu/Amp | 35 | 24.60 | 160 | Promega | Phu/1x/Amp/1x/ | | |
| | | | | | AmpBuf/0.8dNTP/ | | |
| | | | | | 25KCl/10DMF/ | | |
| | | | | | 60 min | | |
| Phu/Amp | 37 | 10.17 | 160 | Promega | Phu/5x/Amp/5x/ | | |
| | | | | | AmpBuf/0.05dNTP/ | | |
| | | | | | 50KCl/10DMF/ | | |
| | | | | | 30 min | | |
| Phu/Amp | 38 | 44.80 | 160 | Promega | Phu/5x/Amp/5x/ | | |
| | | | | | AmpBuf/0.05dNTP/ | | |
| | | | | | 50KCl/10DMF/ | | |
| | | | | | 45 min | | |
| Phu/Amp | 39 | 25.00 | 160 | Promega | Phu/5x/Amp/5x/ | 25.76 | 1.07480231 |
| | | | | | AmpBuf/0.8dNTP/ | | |
| | | | | | 25KCl/10DMF/ | | |
| | | | | | 60 min | | |
| Phu/Amp | 39 | 26.52 | 160 | Promega | Phu/5x/Amp/5x/ | | |
| | | | | | AmpBuf/0.8dNTP/ | | |
| | | | | | 25KCl/10DMF/ | | |
| | | | | | 60 min | | |
| Phu/Amp | 40 | 43.93 | 160 | Promega | Phu/5x/Amp/5x/ | 36.93 | 9.906566 |
| | | | | | AmpBuf/0.8dNTP/ | | |
| | | | | | 25KCl/60 min | | |
| Phu/Amp | 40 | 29.92 | 160 | Promega | Phu/5x/Amp/5x/ | | |
| | | | | | AmpBuf/0.8dNTP/ | | |
| | | | | | 25KCl/60 min | | |
| Phu/Taq | 43 | 37.09 | 160 | Promega | Phu/1x/Taq/ | | |
| | | | | | AmpBuf/0.05dNTP/ | | |
| | | | | | 50KCl/10DMF | | |
| Phu/Taq | 44 | 42.92 | 160 | Promega | Phu/2.5x/Taq/ | | |
| | | | | | TaqBuf/0.8dNTP/ | | |
| | | | | | 30 min | | |
| Phu/Taq | 45 | 39.50 | 160 | Promega | Phu/2.5x/Taq/ | 40.45 | 4.83008627 |
| | | | | | TaqBuf/0.8dNTP/ | | |
| | | | | | 60 min | | |
| Phu/Taq | 45 | 36.16 | 160 | Promega | Phu/2.5x/Taq/ | | |
| | | | | | TaqBuf/0.8dNTP/ | | |
| | | | | | 60 min | | |
| Phu/Taq | 45 | 45.68 | 160 | Promega | Phu/2.5x/Taq/ | | |
| | | | | | TaqBuf/0.8dNTP/ | | |
| | | | | | 60 min | | |
| Phu/Taq | 46 | 42.81 | 160 | Promega | Phu/5x/Taq/ | | |
| | | | | | TaqBuf/0.8dNTP/ | | |
| | | | | | 60 min | | |
| T4/Amp | 16 | 47.44 | 160 | Promega | ThermoT4/1x/ | 35.09 | 9.8006664 |
| | | | | | Amp/2x/T4Buf/ | | |
| | | | | | 1dNTP/0.5NAD/ | | |
| | | | | | 30 min | | |
| T4/Amp | 16 | 28.33 | 160 | Promega | ThermoT4/1x/ | | |
| | | | | | Amp/2x/T4Buf/ | | |
| | | | | | 1dNTP/0.5NAD/ | | |
| | | | | | 30 min | | |
| T4/Amp | 16 | 41.60 | 160 | Promega | ThermoT4/1x/ | | |
| | | | | | Amp/2x/T4Buf/ | | |
| | | | | | 1dNTP/0.5NAD/ | | |
| | | | | | 30 min | | |
| T4/Amp | 16 | 24.55 | 160 | Promega | ThermoT4/1x/ | | |
| | | | | | Amp/2x/T4Buf/ | | |
| | | | | | 1dNTP/0.5NAD/ | | |
| | | | | | 30 min | | |
| T4/Amp | 16 | 43.86 | 160 | Promega | ThermoT4/1x/ | | |
| | | | | | Amp/2x/T4Buf/ | | |
| | | | | | 1dNTP/0.5NAD/ | | |
| | | | | | 30 min | | |
| T4/Amp | 16 | 36.82 | 160 | Promega | ThermoT4/1x/ | | |
| | | | | | Amp/2x/T4Buf/ | | |
| | | | | | 1dNTP/0.5NAD/ | | |
| | | | | | 30 min | | |
| T4/Amp | 16 | 23.06 | 160 | Promega | ThermoT4/1x/ | | |
| | | | | | Amp/2x/T4Buf/ | | |
| | | | | | 1dNTP/0.5NAD/ | | |
| | | | | | 30 min | | |
| T4/Amp | 18 | 34.2 | 160 | Promega | ThermoT4/1x/ | | |
| | | | | | Amp/2x/T4Buf/ | | |
| | | | | | 1dNTP/0.5NAD/ | | |
| | | | | | 60 min | | |
| T4/Amp | 20 | 33.24 | 160 | Promega | ThermoT4/1x/ | 35.73 | 3.13177266 |
| | | | | | Amp/2x/T4Buf/ | | |
| | | | | | 1dNTP/0.5NAD/ | | |
| | | | | | PEG | | |
| T4/Amp | 20 | 40.28 | 160 | Promega | ThermoT4/1x/ | | |
| | | | | | Amp/2x/T4Buf/ | | |
| | | | | | 1dNTP/0.5NAD/ | | |
| | | | | | PEG | | |
| T4/Amp | 20 | 33.02 | 160 | Promega | ThermoT4/1x/ | | |
| | | | | | Amp/2x/T4Buf/ | | |
| | | | | | 1dNTP/0.5NAD/ | | |
| | | | | | PEG | | |
| T4/Amp | 20 | 34.51 | 160 | Promega | ThermoT4/1x/ | | |
| | | | | | Amp/2x/T4Buf/ | | |
| | | | | | 1dNTP/0.5NAD/ | | |
| | | | | | PEG | | |
| T4/Amp | 20 | 37.60 | 160 | Promega | ThermoT4/1x/ | | |
| | | | | | Amp/2x/T4Buf/ | | |
| | | | | | 1dNTP/0.5NAD/ | | |
| | | | | | PEG | | |
| T4/Amp | 21 | 36.10 | 160 | Promega | ThermoT4/1x/ | 36.07 | 5.15506547 |
| | | | | | Amp/4x/T4Buf/ | | |
| | | | | | 1dNTP/0.5NAD | | |
| T4/Amp | 21 | 41.21 | 160 | Promega | ThermoT4/1x/ | | |
| | | | | | Amp/4x/T4Buf/ | | |
| | | | | | 1dNTP/0.5NAD | | |
| T4/Amp | 21 | 30.90 | 160 | Promega | ThermoT4/1x/ | | |
| | | | | | Amp/4x/T4Buf/ | | |
| | | | | | 1dNTP/0.5NAD | | |
| T4/Amp | 57 | 18.07 | 160 | Promega | ThermoT4/1x/ | | |
| | | | | | Amp/2x/T4Buf/ | | |
| | | | | | 1dNTP/PreCRMix | | |
| T4/Amp | 58 | 15.81 | 160 | Promega | ThermoT4/3x/ | | |
| | | | | | Amp/6x/T4Buf/ | | |
| | | | | | 1dNTP/PreCRMix | | |

TABLE 3
Customized SMRT-adapter seqences in IDT compatible format.

| Barcode | | |
|---|---|---|
| Name | Sequence | Barcode Sequene |
| SMRT- | /5Phos/CTG TCT CTT ATA CAC ATC | AGATGTGTATAAGAGACAG |
| A_bc- | TAT CTC TCT CTT TTC CTC CTC CTC | |
| none | CGT TGT TGT TGT TGA GAG AGA | |
| | TAG ATG TGT ATA AGA GAC AG | |
| SMRT- | /5Phos/CTG TCT CTT ATA CAC ATC | CGGAAGAAAGATGTGTATAAGAGACA |
| A_bc001 | TTT CTT CCG ATC TCT CTC TTT TCC | G |
| | TCC TCC TCC GTT GTT GTT GTT | |
| | GAG AGA GAT CGG AAG AAA GAT | |
| | GTG TAT AAG AGA CAG | |
| SMRT- | /5Phos/CTG TCT CTT ATA CAC ATC | GTGTGGAAAGATGTGTATAAGAGACAG |
| A_bc003 | TTT CCA CAC ATC TCT CTC TTT TCC | |
| | TCC TCC TCC GTT GTT GTT GTT | |
| | GAG AGA GAT GTG TGG AAA GAT | |
| | GTG TAT AAG AGA CAG | |
| SMRT- | /5Phos/CTG TCT CTT ATA CAC ATC | TGCGACAAAGATGTGTATAAGAGACAG |
| A_bc006 | TTT GTC GCA ATC TCT CTC TTT TCC | |
| | TCC TCC TCC GTT GTT GTT GTT | |
| | GAG AGA GAT TGC GAC AAA GAT | |
| | GTG TAT AAG AGA CAG | |
| SMRT- | /5Phos/CTG TCT CTT ATA CAC ATC | GCAGCTAAAGATGTGTATAAGAGACAG |
| A_bc010 | TTT AGC TGC ATC TCT CTC TTT TCC | |
| | TCC TCC TCC GTT GTT GTT GTT | |
| | GAG AGA GAT GCA GCT AAA GAT | |
| | GTG TAT AAG AGA CAG | |
| SMRT- | /5Phos/CTG TCT CTT ATA CAC ATC | CCTTAGGAAGATGTGTATAAGAGACAG |
| A_bc011 | TTC CTA AGG ATC TCT CTC TTT TCC | |
| | TCC TCC TCC GTT GTT GTT GTT | |
| | GAG AGA GAT CCT TAG GAA GAT | |
| | GTG TAT AAG AGA CAG | |
| SMRT- | /5Phos/CTG TCT CTT ATA CAC ATC | ACAACGGAAGATGTGTATAAGAGACA |
| A_bc012 | TTC CGT TGT ATC TCT CTC TTT TCC | G |
| | TCC TCC TCC GTT GTT GTT GTT | |
| | GAG AGA GAT ACA ACG GAA GAT | |
| | GTG TAT AAG AGA CAG | |
| SMRT- | /5Phos/CTG TCT CTT ATA CAC ATC | CGATTCGAAGATGTGTATAAGAGACAG |
| A_bc013 | TTC GAA TCG ATC TCT CTC TTT TCC | |
| | TCC TCC TCC GTT GTT GTT GTT | |
| | GAG AGA GAT CGA TTC GAA GAT | |
| | GTG TAT AAG AGA CAG | |
| SMRT- | /5Phos/CTG TCT CTT ATA CAC ATC | CACAGTGAAGATGTGTATAAGAGACAG |
| A_bc014 | TTC ACT GTG ATC TCT CTC TTT TCC | |
| | TCC TCC TCC GTT GTT GTT GTT | |
| | GAG AGA GAT CAC AGT GAA GAT | |
| | GTG TAT AAG AGA CAG | |
| SMRT- | /5Phos/CTG TCT CTT ATA CAC ATC | ATCCTGCAAGATGTGTATAAGAGACAG |
| A_bc015 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | ACGCCATAAGATGTGTATAAGAGACAG |
| A_bc016 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | AGTCGGTAAGATGTGTATAAGAGACAG |
| A_bc017 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | GGCTTGTAAGATGTGTATAAGAGACAG |
| A_bc018 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | TTGGTCAGAGATGTGTATAAGAGACAG |
| A_bc019 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | TAGAGAGGAGATGTGTATAAGAGACAG |
| A_bc020 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | GTTACAGGAGATGTGTATAAGAGACAG |
| A_bc021 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | TTATGCGGAGATGTGTATAAGAGACAG |
| A_bc022 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | TCCACTTGAGATGTGTATAAGAGACAG |
| A_bc023 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | GAATGCACAGATGTGTATAAGAGACAG |
| A_bc024 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | ATGAAGCCAGATGTGTATAAGAGACAG |
| A_bc025 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | GTAGTTCCAGATGTGTATAAGAGACAG |
| A_bc026 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | CTAACGTCAGATGTGTATAAGAGACAG |
| A_bc027 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | AGACACTCAGATGTGTATAAGAGACAG |
| A_bc028 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | CCTTCTTCAGATGTGTATAAGAGACAG |
| A_bc029 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |
| SMRT- | /5Phos/CTG TCT CTT ATA CAC ATC | GAGGTGTTAGATGTGTATAAGAGACAG |
| A_bc030 | TTG CAG GAT ATC TCT CTC TTT TCC | |
| | TCC TCC TCC GTT GTT GTT GTT | |
| | GAG AGA GAT ATC CTG CAA GAT | |
| | GTG TAT AAG AGA CAG | |

### Example 2: Methods

### Cell Lines and Cell Culture

OS152 osteosarcoma cells were routinely tested for authenticity and mycoplasma via CellCheck 9 Plus (IDEXX BioAnalytics). Cells were cultured in standard 1×DMEM (Gibco) supplemented with 10% Bovine Growth Serum (HyClone) and 1% 100×Penicillin-Streptomycin-Glutamine (Corning). E14 mouse embryonic stem cells (mESC E14) were a gift from Elphege Nora (UCSF) and were routinely tested for mycoplasma via PCR (NEBNext® Q5 2×Master Mix). Feeder-free cultures were maintained on 0.2% gelatin, in KnockOut DMEM 1×(Gibco) supplemented with 10% Fetal Bovine Serum (Phoenix Scientific), 1% 100×GlutaMAX (Gibco), 1% 100×MEM Non-Essential Amino Acids (Gibco), 0.128 mM 2-mercaptoethanol (BioRad), and purified 1×Leukemia Inhibitory Factor (gifted by Barbara Panning, UCSF). Cultures were passaged at least twice before use.

### Human Subjects

De-identified primary tumor and metastatic lymph node tissue used to generate PDX models were donated by a patient who provided written informed consent under UCSF IRB protocol 11-05226.

### Assembly of Hairpin Adaptor Loaded Tn5 Transposomes and Assays for Transposase Activity

### Annealing Adaptors

HPLC-purified uniquely barcoded (Hamming distance ≥4) hairpin oligonucleotides were purchased from IDT (Coralville, IA) and normalized to 100 μM in RNase-free water. Adaptors were diluted 20 to 20 μM in 1×Annealing Buffer (10 mM Tris-HCl pH 7.5 and 100 mM NaCl), annealed via thermocycler (95° C. 5 minutes, 25° C. 30 minutes, 4° C. hold), and rapidly cooled to −20° C. for long-term storage.

Loading Tn5 Transposases with SMRT-Tag Adaptors

Purified triple mutant Tn5R27S, E54K, L372P enzyme (Tn5) was obtained from the QB3 MacroLab (UC Berkeley). Frozen aliquots of stock Tn5 enzyme (3.9 mg/mL) suspended in Storage Buffer (50 mM Tris-HCl pH 7.5, 800 mM NaCl, 0.2 mM EDTA, 2 mM DTT, 10% glycerol) were thawed at 4° C., diluted in Tn5 Dilution Buffer (50 mM Tris-HCl pH 7.5, 200 mM NaCl, 0.1 mM EDTA, 2 mM DTT, and 50% glycerol) to ˜1 mg/mL Tn5 (18.9 μM monomer) by rotational mixing at 4° C. for 3.5 h until fully homogenized. Tn5 was loaded with hairpin adaptors by gentle mixing of 1.02×volumes of 1 mg/mL Tn5 with 1×volume of 20 μM annealed adaptors using a wide-bore pipette, followed by incubation at 23° C. with continuous agitation at 350 rpm for 55 minutes. Loaded Tn5 (9.4 μM monomer) supplemented with glycerol to a final concentration of 50% can be stored at −20° C. for up to 6 months.

### Confirming Tn5 Loading

Effective adaptor loading was confirmed by blue native PAGE gel-electrophoresis. Briefly, 1-2 μL of loaded Tn5 stock (9.4 μM monomer) diluted in Native Gel Loading Buffer (Invitrogen) was loaded per well on a NativePAGE 4-16% Bis-Tris Gel (Invitrogen) and run at 150V for 1 hour at 4° C., followed by 180V for 15 min. Gels were stained with 1×SYBR Gold Solution (Invitrogen) in 1×TAE, followed by 1×Coomassie Blue (Invitrogen) for 1 hour at room temperature, and imaged on an Odyssey XF imaging system (LI-COR, software version 1.1.0.61).

### Assessing Tunability of Fragment Lengths

Tagmentation optimization was carried out using serially diluted hairpin-loaded Tn5 stock (9.4 μM monomer) in RNase-free water. Diluted transposomes were incubated with 160 ng of human gDNA (Promega) while varying buffers, temperatures, and incubation times. Reactions were terminated with 0.2% SDS (final concentration 0.04%). Analytical electrophoresis was performed on a 0.4-0.6% 1×-TAE-agarose gel with 2-3 hour run time at 60-80V to resolve bands. Gels were stained with 1× SYBR Gold and imaged on an Odyssey XF imaging system.

### SMRT-Tag of Genomic DNA

### Preparation of SMRT-Tag Libraries

Purified high molecular weight gDNA (HG002, HG003, and HG004; Coriell

Institute) was normalized to 40-50 160 ng per sample as input for library preparation, which included tagmentation, gap repair, exonuclease cleanup and validation steps. Tagmentation reactions were prepared by diluting each sample up to 9 μL in 1×Tagmentation Mix (10 mM TAPS-NaOH pH 8.5, 5 mM MgCl2, and 10% DMF) and adding 1 μL of barcoded Tn5 (varying dilutions from stock). Reactions were incubated at 55° C. for 30 minutes and terminated by adding 0.2% SDS (final concentration 0.04%) prior to room temperature incubation for 5 minutes, 2× SPRI cleanup, and elution in 12 μL of 1× elution buffer (EB, 10 mM 5 Tris-HCl pH 8.5). Tagmented samples were gap repaired at 37° C. for 1 hour in Repair Mix (2U Phusion-HF, 80U Taq DNA Ligase, 1×Taq DNA Ligase Reaction Buffer, and 0.8 mM dNTPs [New England Biolabs, NEB]). Samples were cleaned up using 2×SPRI beads and eluted in 12 μL of 1×EB. For exo digestion, reactions were incubated in ExoDigest Mix (100U NEB Exonuclease III per 160 ng, 1×NEBuffer 2) at 37° C. for 1 hour, followed by 2×SPRI cleanup and elution in 12 μL of 1×EB. Libraries prepared for method optimization were multiplexed and pooled at equimolar concentrations measured by Qubit 1×High Sensitivity DNA Assay (Thermo Fisher Scientific).

### Titration of Transposome Concentrations and Input Amounts at Varying Temperatures

To characterize the tunability of SMRT-Tag, tagmentation reactions were carried out essentially as described using serially diluted hairpin-loaded Tn5 stock (9.4 μM monomer) in RNase-free water. Diluted transposomes (0.05, 0.50, and 5 pmol monomer) were combined with 40, 200, and 1,000 ng of HG003 gDNA (Coriell Institute) and incubated at 37° C. or 55° C. for 30 minutes. Gap repair, exo cleanup, library validation, and multiplexing were performed as above.

### SMRT-Tag Library Quality Control

To assess repair efficiency (i.e., the extent to which tagmented DNA is converted to sequenceable library molecules) 1 μL of eluted library before and after treatment with ExoDigest mix was measured by Qubit 1× High Sensitivity DNA Assay. To validate library quality, 1 μL of eluted library was analyzed via Qubit 1×High Sensitivity DNA and Agilent 2100 Bioanalyzer High Sensitivity DNA Assays to measure sample concentration and size distribution, respectively.

### Assaying Barcode Hopping Via Pooled Gap Repair

To assess whether gap repair affected sample barcoding, SMRT-Tag libraries were prepared as described using barcoded hairpin-loaded Tn5, but samples were pooled after tagmentation into a single gap repair reaction. After gap repair, the pooled sample was treated with ExoDigest mix as described to produce a single pooled library.

### Optional Size Selection of SMRT-Tag Libraries

For a subset of libraries, size selection using 35% (v/v) AMPure PB beads diluted in 1×EB was performed to enrich for molecules >5-kb (HMW). 3.1×volumes AMPure PB beads were added to a library, incubated at room temperature for 15 minutes and washed twice with 80% ethanol for 1 minute. The size selected HMW fraction was eluted in 15μL of 1×EB. Additionally, for some libraries, 0.25×AMPure PB cleanup of the sCLpernatant was used to recover the low molecular weight fraction (LMW, <5-kb), which was then eluted in 15 μL of 1×EB.

### Sequencing SMRT-Tag Libraries

SMRT-Tag libraries were sequenced on a PacBio Sequel II using 8M SMRTcells with or without multiplexing. For each SMRTcell, movies were collected for 30 hours, with a 2-hour pre-extension time and a 4-hour immobilization time. Both 2.1 and 2.2 polymerases were used, with polymerase choice dependent on average library size (e.g., HMW fractions were sequenced with 2.2 polymerase while 2.1 polymerase was used for LMW fractions and libraries without size selection).

### SAMOSA-Tag of Cell Lines

### Nuclei Isolation

1-2 million OS152 or mESC E14 cells were harvested by centrifugation (300×g, 4° C., 10 minutes), washed in cold 1× PBS, and resuspended in 1 mL cold Nuclear Lysis Buffer (20 mM HEPES, 10 mM KCl, 1 mM MgCl2, 0.1% Triton X-100, 20% Glycerol, 1×Protease Inhibitor [Roche]) by gentle mixing with a wide-bore pipette tip. The suspension was incubated on ice for 5 minutes, then nuclei were pelleted (600×g, 4° C., 10 minutes), washed with Buffer M (15 mM Tris-HCl pH 8.0, 15 mM NaCl, 60 mM KCl, 0.5 mM Spermidine), and counted on a Countess III cell counter (Thermo Fisher Scientific).

### In Situ SAMOSA Footprinting

Permeabilized nuclei were pelleted (600×g, 4° C., 10 minutes) and resuspended in 400 μL Buffer M supplemented with 1 mM S-adenosyl-methionine (SAM, New England Biolabs) and 200 μL was reserved as an unmethylated control. Nonspecific adenine methyltransferase EcoGII (250U, 10 μL of 25,000 U/mL stock, New England Biolabs) was added to the reaction and incubated at 37° C. for 30 minutes with 300 rpm shaking every 2 minutes. SAM was replenished to 1.16 mM after 15 minutes in the methylation reaction and unmethylated control.

### Tagmentation of Footprinted Nuclei

Methylated nuclei and unmethylated controls were pelleted by centrifugation (600×g, 10 minutes) and gently resuspended in 250 μL 1×Omni-ATAC Buffer (10 mM Tris-HCl pH 7.5, 5 mM MgCl2, 0.33×PBS, 10% DMF, 0.01% Digitonin [Thermo Fisher Scientific], 0.1% Tween-20). The nuclei suspension was then filtered through a 40 μm cell strainer (Scienceware FlowMi), and dissociation of aggregates was verified by counting and visualization on a Countess III cell counter. Both methylated and unmethylated reactions were split into 10,000-50,000 nuclei aliquots and, based on the desired library size and cell type, 9.4-18.8 pmol of uniquely barcoded Tn5 was added per reaction. Tagmentation reaction volumes were brought up to 50 μL in 1× Omni-ATAC Buffer, then incubated at 55° C. for 45-60 minutes.

### Tagmentation Termination and DNA Purification

To terminate tagmentation, reactions were first treated with 10 μL of 10 mg/mL RNase A (Thermo Fisher) at 37° C. for 15 minutes with 300 rpm shaking. Termination Lysis Buffer (2.5 μL of 20 mg/mL Proteinase K [Ambion], 2.5 μL of 10% SDS and 2.5 μL of 0.5M EDTA) prepared at room temperature was added to the reaction, followed by incubation at 60° C. with 1000 rpm continuous shaking for at least 1 hour and up to 2 hours for improved lysis. To extract tagmented fragments, 2×SPRI beads were added, mixed until homogenous, and incubated at 23° C. for 30 minutes with mixing at 350 rpm every 3 minutes to keep beads dispersed. Beads were pelleted via magnet, washed twice in 80% ethanol for 1 minute, then eluted in 20 μL of 1× EB at 37° C. for 15 minutes with interval mixing at 350 rpm every 3 minutes to maximize sample recovery. An additional 0.6×SPRI cleanup was used to enrich for fragments >500 bp. Samples were stored at 4° C. overnight, or up to two weeks at −20° C.

### Preparation of SAMOSA-Tag Libraries

Purified, tagmented DNA extracted from methylated nuclei or unmethylated controls was normalized up to 160 ng per sample as input for SAMOSA-Tag library preparation. For both OS152 and mESC E14 cells, a total of 8 methylated replicates along with unmethylated controls, each tagmented with a different set of barcoded hairpin adaptors, were processed in subsequent steps, including gap repair, exonuclease cleanup and library validation. For gap repair, tagmented samples were incubated in Repair Mix (2U Phusion-HF, 80U Taq DNA Ligase, 1×Taq DNA Ligase Reaction Buffer, 0.8 mM dNTP mix) at 37° C. for 1 hour, followed by 2×SPRI cleanup and elution in 12 μL of 1×EB. For exonuclease cleanup, reactions were incubated in ExoDigest Mix (100U Exonuclease III per 160 ng, 1× NEBuffer 2) at 37° C. for 1 hour, followed by 2×SPRI cleanup and elution in 12 μL of 1×EB. Repair efficiency and library quality were assessed as for SMRT-Tag.

### Ex Situ SAMOSA-Tag

Permeabilized mESC E14 nuclei were subjected to SAMOSA footprinting as above. After the methylation reaction, 10 μL of RNaseA (10 mg/mL) was added and incubated at 37° C. for 15 minutes. Then, 2.65 μL of 10% SDS and 2.65 μL of 20 mg/mL Proteinase K (Thermo Scientific) were added, and the solution was incubated at 65° C. for 3 hours. For DNA extraction, an equal volume of phenol: chloroform: isoamyl Alcohol (25:24:1, v/v) was added and vigorously mixed by shaking. Samples were centrifuged at maximum speed (16,000×g) for 2 minutes at room temperature. The aqueous phase was removed and 0.1× volume of 3M NaOAc, 1 μL of GlycoBlue coprecipitant (Invitrogen), and 3× volumes of cold 100% ethanol were added, mixed by inversion, and incubated overnight at −80° C. Samples were centrifuged at maximum speed for 30 minutes at 4° C., followed by a wash with 500 μL 70% ethanol and spun at maximum speed for 2 minutes at 4° C. The resulting pellet was air dried and resuspended in 40 μL of 1×EB. Sample concentrations were measured via Qubit High Sensitivity DNA Assay and DNA quality was checked on the Agilent 2200 TapeStation system. 100 ng 5 of purified SAMOSA gDNA was used for library preparation. Tagmentation was performed with a normalized amount of Tn5 (0.046 pmol monomer), followed by gap repair, exonuclease cleanup and library validation.

### Sequencing SAMOSA-Tag Libraries

SAMOSA-Tag libraries were multiplexed and sequenced on PacBio Sequel II 8M SMRTcells using 2.1 or 2.2 polymerase chemistry depending on the sample. For each SMRTcell, movies were collected for 30 hours with a 2-hour pre-extension time and a 4-hour immobilization time.

### SAMOSA-Tag of Prostate Cancer Patient Derived Xenografts (PDX)

### Prostate Cancer PDX Generation and Characterization

Patient derived xenograft (PDX) models were generated as previously

described³⁸. Briefly, 3-5 mm tumor fragments were isolated from a primary prostate (Gleason 9) tumor and synchronous metastatic lymph node from the same patient. This patient initially presented with high-risk prostate cancer (pre-treatment PSA 19.1 ng/ml, Gleason 4+5, T3aN1M0) with bilateral external pelvic lymph nodes 6-9 mm metastases on PSMA PET scan. Samples were obtained during robotic prostatectomy and pelvic lymph node dissection. Tumor fragments were taken immediately after prostatic devascularization during surgery to minimize cell death while preserving the integrity of the tumor microenvironment, placed in 10 mL of RPMI 1640 medium for short transport to the lab from the operating room, and implanted subcutaneously into the flank of NSG mice to establish PDX lines. PDX tumors were cryopreserved for future experiments after three passages in NSG mice. To ensure that PDXs faithfully capture the heterogeneity of prostate cancer, tumor sections were subjected to histopathological comparison after each passage. To confirm the passaged PDXs maintained the integrity of the original PDX, growth patterns were examined. Passage 10 PDXs were processed via SAMOSA-Tag.

### PDX Sample Collection and Processing

On the day of collection, tumors were surgically explanted from PDX mice, aiming to minimize residual mouse tissue, and immediately placed into sterile collection buffer (RPMI-1640) on ice. For each sample, the tumor mass was manually cut to aid dissociation using surgical blades (Fisher Scientific). Samples were placed intomdigestion buffer (amount per sample: 5 mL of F-12K [Fisher Scientific]; 5 mL of DMEM [Fisher Scientific]; 10 μL DNAseI [Worthington Biochemical]; 10 mg of Liberase-TL [Sigma-Aldrich]; 65 mg of Collagenase Type III [Worthington Biochemical]; 100 μL of 100×Penicillin-Streptomycin [Thermo Fisher Scientific]; 40 μl of 0.25 mg/mL. Amphotericin B [Fisher Scientific]) and shaken at 750 rpm, 37° C. for 1 hour until clumps were visibly dissociated. The resulting single-cell suspensions were spun at 4° C. for 5 minutes at 800×g and the pellets resuspended in cold 1 mL PBS (Sigma-Aldrich). Cell suspensions were strained through a Falcon 70 μm cell strainer (Corning) using a wide-bore P1000 filter tip. Samples were washed twice in 1×PBS and pelleted via centrifugation at 4° C. for 5 minutes at 800×g. The resulting pellet was resuspended in 1 mL Cell Staining Buffer (Biolegend). Cell counts by hemocytometer were ˜8-12.5×10⁶ cells/mL.

### Antibody Staining and FACS Enrichment of Live, Human Cells

For blocking, 20 μL of Human TruStain FcX (BioLegend) was added to each sample and incubated for 10 minutes at 4° C. in the dark. 1 μg of PE anti-mouse H-2 Antibody (BioLegend, Cat. 125505) was added per 8-12.5×10⁶ cells and incubated for 25 minutes at 4° C. in the dark. Cells were washed twice in Cell Staining Buffer and pelleted at 4° C., 350×g. Cells were then incubated with 1 μL SYTOX Red Dead Cell Stain (Thermo Fisher Scientific) for 15 minutes at 4° C. in the dark. Cells were kept foil-covered on ice until sorting. To remove contaminant mouse and dead human cells, PDX-derived cells were sorted using a BD FACS Aria II running FACS DIVA software (BD Biosciences) at the UCSF Center for Advanced Technology. Visualization and analysis of FACS data was performed in FlowJo (v10.8.2, BD Biosciences). Cell singlets were selected by gating on forward scatter. Live human cells were selected as PE negative and APC negative, calibrated against single-stain controls, and collected into a 15 ml conical tube containing 1 mL of 1×PBS. Collection tubes were rinsed with 500 μL of 1×PBS to maximize recovery. Cell counts via hemocytometer were between 1.20-1.75M cells per PDX sample.

### SAMOSA-Tag of PDX Cells

Sorted cells were placed on ice and immediately processed via in situ SAMOSA-

Tag as described for OS152 and mESC E14 cells, with spin speed reduced from 600×g to 400×g. Due to significant cell loss during preparation, only two unmethylated controls were generated for the primary PDX, and one unmethylated control for the metastasis. Resulting SAMOSA-Tag libraries were assayed for quality as described above. Primary and metastasis PDX libraries were separately pooled and sequenced each on 1 SMRTcell 8M using 2.1 polymerase chemistry, and the same sequencing parameters as for OS152 and mESC E14 in situ SAMOSA-Tag libraries.

### Ligation-Based Library Preparation

Low Input gDNA Libraries

Conventional SMRTbell libraries were prepared from high molecular weight (HMW) HG002 gDNA (Coriell Institute) using the PacBio SMRTbell Express Template Prep Kit 2.0 protocol (TPK2.0) according to the manufacturer's instructions. To assess the efficiency of the enzymatic ligation step, 40 ng of sheared gDNA wasused as input. Briefly, the TPK2.0 protocol consists of removal of single stranded overhangs, DNA damage (PreCR) repair, end-repair, A-tailing, barcoded SMRTbell adapter ligation, and exo digestion followed by 1× AMPure PB bead cleanup. Final sample concentration was measured via Qubit High Sensitivity DNA Assay. Across replicates, insufficient library was obtained to proceed with sequencing. DNA extraction and preparation of high-input TPK2.0 libraries sequenced at low OPLC Bulk gDNA was extracted from mESC E14 cells via phenol: chloroform: isoamyl alcohol extraction as described for ex situ SAMOSA-Tag. Sample concentration was measured by Qubit High Sensitivity DNA Assay. Approximately 2.5 μg purified DNA was fragmented to 6-8 kb using a g-TUBE (PN: 520079, Covaris) with an Eppendorf 5424 rotor spun at 7,000 rpm for 6 passes. Sheared DNA was used as input for the TPK2.0 protocol as above. The resulting library was assayed via Qubit 1×High Sensitivity DNA Assay and Agilent 2100 Bioanalyzer High Sensitivity DNA Assay to determine concentration and size. An aliquot of the library was loaded at 44.6 pM on a SMRTCell 8M and sequenced on a PacBio Sequel II for 30 hours with a 2-hour preextension time. This confirmed that high-input TPK2.0 libraries can be sequenced at low OPLC.

### Estimating Reaction Efficiency

Multiple measures of reaction efficiency were calculated. Tagmentation, gap repair, and exonuclease stepwise efficiencies were determined by dividing the output mass of a given step in nanograms by the input mass in nanograms for that same step. The term “repair efficiency” was used to describe the efficiency of the exonuclease cleanup step, as a proxy for effectiveness of gap repair and conversion of hairpin-tagmented DNA into sequenceable library. Overall reaction efficiency was either estimated by comparing the final amount of library versus input, or, for libraries where per-step efficiencies were calculated, by multiplying the three stepwise efficiencies together.

### Data Preprocessing

For all experimental data, HiFi reads were generated from raw subreads using ccs (v.6.4.0, Pacific Biosciences) with the additional flag—hifi-kinetics to annotate reads with kinetic information. Lima (v.2.6.0, Pacific Biosciences) with fla—ccs was used to demultiplex runs into sample-specific BAM files, and samples sequenced across multiple cells were merged using pbmerge (v1.0.0, Pacific Biosciences). Reads were aligned using pbmm2 (v.1.9.0, Pacific Biosciences) to the relevant reference genome. SMRT-Tag reads were aligned to the hs37d5 GRCh37 reference genome for variant analyses, and the hg38 reference genome for all other analyses. OS152 SAMOSA-Tag reads were aligned to the hg38 reference genome. mESC E14 in situ and ex situ SAMOSA-Tag reads were aligned to the GRCm38 reference genome. Primary and metastasis PDX SAMOSA-Tag reads were aligned to a joint hg38/GRCm39 reference genome and only reads uniquely aligning to hg38 retained for downstream analyses. For all reads, read quality was ascertained from the ccs estimates, and empiric per-read quality score (Q-score) was calculated as −log10 (1−(n_{matches}/(n_{matches}+n_{mismatches}+n_{del}+nins)) or the maximal theoretical quality score if the read contained no sequence variation.

### SNV-Based Analysis of SMRT-Tag Semultiplexing

The hs37d5 GRCh37 reference genome³⁹, GIAB v4.2.1 benchmark⁴⁰ VCF and BED files for HG002, HG003, and HG004, and GIAB v3.0 GRCh37 genome stratifications²⁵ were accessed as follows:

trace.ncbi.nlm.nih.gov/giab/ftp/release/references/GRCh37/hs37d5.fa.gz.

ncbi.nlm.nih.gov/giab/ftp/release/AshkenazimTrio/HG002_NA24385_son/NISTv 4.2.1/GRCh37.

ncbi.nlm.nih.gov/giab/ftp/release/AshkenazimTrio/HG003_NA24149_father/NIS Tv4.2.1/GRCh37.

ncbi.nlm.nih.gov/giab/ftp/release/AshkenazimTrio/HG004_NA24143_mother/NI STv4.2.1/GRCh37.

ncbi.nlm.nih.gov/giab/ftp/release/genome-stratifications/v3.0/v3.0-stratifications-GRCh37.tar.gz

Private SNVs for each individual were obtained using bcftools (v1.15.1) and regions for variant calling and evaluation comprising the union of the benchmark BED files were generated using bedtools (v2.3.0).

Demultiplexed HG002, HG003, and HG004 SMRT-Tag reads were aligned to hs37d5 using the minimap2 aligner (v2.15) implemented in pbmm2 (v1.9.0) and per-base coverage was tabulated using mosdepth (v0.3.3).

Given low depth of coverage, we naively called SNVs within regions defined in the GIAB benchmark BED files supported by at least 2 reads and with minimum mapping quality of 15 using samtools mpileup (v1.15.1) and a custom script.

For each of HG002, HG003, and HG004, naïve SNV calls were intersected with private benchmark SNVs in regions labeled ‘not difficult’ in the GIAB v3.0 genome stratification and covered by at least 2 SMRT-Tag reads using bedtools (v2.30.0), samtools (v1.15.1), and bcftools (v1.15.1).

### HG002 Small Variant (SNV and Indel) Calling and Benchmarking

In addition to the hs37d5 GRCh37 reference genome, GIAB v4.2.1 benchmark VCF and BED files for HG002, and GIAB GRCh37 v3.0 genome stratifications used in the genotype demultiplexing analysis, we downloaded publicly available HG002 PacBio Sequel II HiFi reads (SRX5527202), which were generated with ˜11 kb size selection and Sequel II chemistry 0.9 and SMRTLink 6.1 pre-release, and are available aligned to the same reference genome via GIAB.

Pbmm2 was used for alignment of HG002 SMRT-Tag CCS reads to hs37d5 as before. Similarly, median total coverage for SMRT-Tag and GIAB PacBio reads was determined using mosdepth. CCS reads were subsampled to 3-, 5-, 10-, and 15-fold depths using samtools (v1.15.1) based on mosdepth median coverage.

Small variants (SNVs and indels) were called using DeepVariant (v1.4.0). Variants were then compared called from SMRT-Tag and HG002 PacBio Sequel II HiFi data against GIAB/NIST v4.2.1 benchmarks2 using hap.py (v0.3.12) and GIAB v3.0 GRCh37 genome stratifications.

### Structural Variant Calling and Benchmarking

HG002 SMRT-Tag and GIAB Sequel II data were pre-processed as described above for small variant detection. Benchmark NIST Tier 1 SV calls for HG002 (v0.6) and tandem repeats for hg19/hs37d5 were obtained from:

ncbi.nlm.nih.gov/ReferenceSamples/giab/release/AshkenazimTrio/HG002_NA24 385_son/NIST_SV_v0.6/HG002_SVs_Tier1_v0.6.bed

ncbi.nlm.nih.gov/ReferenceSamples/giab/release/AshkenazimTrio/HG002_NA24 385_son/NIST_SV_v0.6/HG002_SVs_Tier1_v0.6.vcf.gz

hgdownload.soe.ucsc.edu/goldenPath/hg19/bigZips/hg19.trf.bed.gz.

Reads were subsampled as described above for small variant analysis. Structural variants were called using pbsv (v2.8.0; github.com/PacificBiosciences/pbsv).

VCF files output by pbsv were compressed and indexed using samtools. Variants were then benchmarked against the NIST v0.6 Tier 1 structural variant calls for HG002 using Truvari (v3.3.0)⁵⁰.

### Predicting CpG Methylation in Single Molecule Reads

HiFi reads produced using 2.1 and 2.2. polymerase chemistries were demultiplexed with lima (v.2.6.0) to remove barcode sequences. Primrose (v.1.3.0, Pacific Biosciences; now Jasmine) was used to predict m⁵dC methylation status at CpG dinucleotides. Methylation probabilities encoded using the BAM tags ML and 5 MM were parsed to continuous values for downstream single-molecule methylation predictions. Per-CpG methylation was estimated using tools available at github.com/PacificBiosciences/pb-CpG-tools.

### Predicting Micleosome Footprints in SAMOSA-Tag Data

SAMOSA-Tag data were preprocessed as above and analyzed using a computational pipeline for detecting m⁶dA methylation in HiFi reads³¹. In brief, per-read kinetics of polymerase base addition were extracted, and a series of neural networks trained on kinetic measurements from methylated and unmethylated controls were used to predict the probability of m⁶dA methylation at all adenines on the forward and reverse strands. Methylation probabilities were binarized into accessibility calls using a two-state hidden Markov model. Accessibility information was encoded for each read as a 0/1 modification probability using the BAM tags MM and ML for visualization with a modified version of IGV.

### Comparing ATAC-Seq and SAMOSA-Tag

Total SAMOSA accessibility and normalized ATAC-seq signal were aggregated at ATAC-seq peaks identified in the OS152 cell line. Values were log-transformed and Pearson's r was calculated as a measure of correlation.

### U2OS and LNCaP CTCF ChIP-Seq Processing

Processed BED files from published ChIP-seq in U2OS cells³⁴ (GEO accession GSE87831) and the metastatic prostate adenocarcinoma cell line LNCaP51 (ENCODE accession ENCFF275GDH) were lifted over from reference hg19 to hg38 and then analyzed as previously described42 to obtain predicted binding sites.

### Insertion Preference Analyses at TSS and CTCF Sites

Read-ends from SAMOSA-Tag data were extracted from BAM files and tabulated in a 5-kb window surrounding annotated GENCODEV28 (hg38) or GENCODEM25 (GRCm38) transcriptional start sites (TSSs) or ChIP-seq backed CTCF motifs. For visualization, all metaplots were smoothed with a running mean of 100 nucleotides. FRITSS/FRICBS was calculated as the fraction of read ends falling within the 5-kb window.

CTCF CpG and Accessibility Analyses m⁶dA accessibility signal around predicted CTCF sites was extracted from pickle files storing serialized data and Leiden clustered as described³¹. In addition to filtering out clusters that together accounted for less than 10% of data, a cluster of completely unmethylated fibers were manually filtered out. Compared against analyzed fibers surrounding CTCF sites, this cluster accounted for 3,627 fibers, or 11.5% of all CTCF-motif containing fibers in OS152 SAMOSA-Tag, and 245 fibers or 1.5% in PDX SAMOSA-Tag. For CpG analyses, custom Python scripts were used to convert CpG methylation to similar format as medA accessibility and extracted CpG methylation per molecule centered at CTCF sites. Data were then converted into text files for visualization in ggplot2.

### Classifying Fibers by CpG Content and CpG Methylation

Fibers were binned by CpG content and CpG methylation to define four classes: high CpG content/methylation (i.e., >0.5 average primrose score on a fiber; >10 CpGs per kilobase), low CpG content/methylation (vice-versa), as well as high/low and low/high bins.

### Fiber Type Clustering

Single-molecule accessibility autocorrelations were calculated and Leiden clustering was performed as described previously31. In addition to filtering out clusters that together comprised less than 10% of all fibers, unmethylated/lowly methylated fibers were also manually filtered out, which fell out of the Leiden clustering analysis and together accounted for 317,768 fibers (12.5% of all clustered fibers) in OS152 SAMOSA-Tag data.

### Fiber Type Enrichment

Fisher's exact tests to determine fiber type enrichment were performed as previously reported³¹. Briefly, to examine enrichment of fiber type A stratified by feature B, a 2×2 contingency table was constructed by counting fibers that fell into four groups: A∩B, A∩B′, A′∩B, and A′∩B′. The table was used as input for a one sided Fisher's exact test and resulting p-values were corrected for multiple testing using Storey's q-value.

### Prostate-Specific Epigenome Stratification

Normal prostate tissue-specific chromHMM annotations in BED format were

previously reported41 (NGDC accession OMIX237-64-02) and were lifted over from reference hg19 to hg38.

### Differential Fiber Usage Calculation

Differential fiber usage per domain was determined using a logistic regression

framework. First, coverage of epigenomic domains by different fiber types in each replicate was calculated as described³¹. To determine differential usage for fiber type A in domain B, coverage was aggregated by whether individual fibers were of type A and mapped to domain B. Counts for these two categories—domain A∩fiber B vs. (domain A∩fiber B)′ were determined for each replicate, and then normalized across replicates using a median of medians approach to account for library depth. Normalized counts per replicate were used as weights for a logistic regression model with the domain/fiber status as the response variable and case status of the library (primary vs. metastasis) as the predictor. The glm function in R (v.4.2.1) was used to fit the model and the coefficient of case status was used as an estimate of log fold change (Δ) in metastasis vs. primary. This regression was repeated for every observed domain and fiber combination (7 fiber types, and 17 domain annotations), and the associated fold change p-values were corrected for multiple testing using Storey's q-value52. The threshold for significance was set at q≤0.05.

### Experimental Design Considerations for PacBio Sequencing

The PacBio single-molecule sequencing (SMS) platform is fundamentally different from the Illumina and Oxford Nanopore instruments. There are several technical considerations particular to PacBio SMS 5 that motivated our experimental design for developing and optimizing SMRT-Tag and SAMOSA-Tag. Leveraging the potential of PacBio sequencing (namely, direct detection of DNA modifications), requires libraries be made without PCR. This leads to a critical limitation, as DNA is lost at every step of library preparation. Importantly, this includes steps required for loading the PacBio sequencer—specifically, polymerase binding and loading on flow cells (SMRTCells). PacBio SMS performance is influenced by several properties: library fragment length distribution, presence of DNA damage, batch-to-batch SMRTCell and polymerase characteristics, and perhaps most importantly, the on-plate loading concentration (OPLC) of libraries. Maximizing the P1 productivity (fraction of zero-mode waveguides sequencing one and only one molecule) and CCS yield (and thus, minimizing cost-per base) of a PacBio flow cell requires a high per-run OPLC. The only ways to maximize OPLC are by (i) minimizing DNA loss during clean-up steps and (ii) pooling barcoded libraries together when possible. We provide salient technical details including OPLC for all SMRT-Tag and SAMOSA-Tag libraries sequenced in this study. While achieving high OPLC to minimize cost-per-base was the primary focus of most experiments presented in this paper, as a valuable reference point an experiment was included where a single library from 40 ng of human gDNA was tagmented and sequenced on a single SMRTCell (FIGS. 2A-2G). This serves to illustrate the capability of SMRT-Tag for maximizing coverage of low-input samples.

### Comparison of Input DNA Requirements for SMRT-Tag, SAMOSA-Tag, and Other Methods

SMRT-Tag and SAMOSA-Tag input reduction relative to other methods was estimated based on the following:

The standard ligation-based PacBio Template Prep Kit 2.0 recommends minimum input of 5 μg DNA, whereas the SMRTbell Prep Kit 3.0 (released in mid-2022) recommends 1-5 μg (˜170,000-800,000 human cells). Taking 40 ng (˜7,000 human cells) as a conservative lower bound for SMRT-Tag, the input required relative to ligation-based methods is 0.8-4%, representing reduction of 96-99.2%.

The input amounts reported in the publications describing single-molecule chromatin profiling methods are: SAMOSA4,37/Fiber-seq5 (2 μg), DiMeLo-seq8 (6-30 μg), SMAC-seq6 (6 μg), nanoNOMe7 (2-3 μg), and MeSMLR-seq12 (quantity not reported, but minimum quoted for the ONT Ligation Sequencing Kit is 1 μg). SAMOSA-Tag experiments used 30,000-50,000 nuclei (˜180-300 ng DNA). Noting that direct comparison is challenging given that the substrate for SAMOSA-Tag is chromatin and not purified DNA, the input required relative to other chromatin profiling methods is 0.6-9%, representing reduction of 91-99.4%.

Accordingly, it was conservatively estimated that SMRT-Tag requires 1-5% as much DNA as ligation-based library preparation (equating to reduction by 95-99%) and SAMOSA-Tag requires 1-10% of the input reported for comparable methods (corresponding to reduction by 90-99%). Therefore, SMRT-Tag and SAMOSA-Tag reduce the magnitude of input required by approximately 1 or 2 orders (i.e., 10-fold or 100-fold).

### Molecule Length and Molarity

In preparing a PacBio library of a given mass, the number of molecules is inversely proportional to the fragment length. Given mass m in nanograms and length L, the number of picomoles of DNA can be estimated as, e.g., m×10³/(660×N) where 660 pg/pmol is the average molecular weight of a base pair. Therefore, tagmenting gDNA into very long fragments may yield a library below the on plate loading concentration (OPLC) lower bound of 20-40 pM (i.e., 2.3-4.6 fmol in a 115 μuL volume) for Sequel II SMRTCells. On the other hand, if input DNA is not limiting, it may be reasonable to target longer fragments. Based on the mean library conversion efficiency of ˜20% and the relationship between mass and length of DNA, the input required for a particular library size can be readily estimated. For example, to achieve an OPLC of 37 PM (volume: 115 μL) for libraries with median lengths of 2.3, 10, and 100 kb, the starting material required is approximately 35, 150, and 1,500 ng, respectively. Considerations related to length and molar quantity are not unique to PacBio sequencing. For the Oxford Nanopore Rapid sequencing kit (Cat. No. SQK-RAD114), which uses a transposase-based approach to reduce input requirement to 50-100 ng, multiplexing is often required to reduce per-sample cost.

Input DNA quality

PacBio's sequencing-by-synthesis chemistry relies on processive polymerization on a native, circular template. High-quality DNA is therefore required for PacBio HiFi or circular consensus sequencing (CCS). Ideal input is high molecular weight (HMW) DNA. There are several approaches for assessing input quality. Automated (e.g., Agilent Femto Pulse) or manual (e.g., BioRad CHEF-DR II) pulsed field gel electrophoresis systems are the gold²⁵ standard but can be cumbersome. Alternatively, 10-25 ng DNA loaded on a 0.4-0.6% TAE/agarose gel run at low voltage (60-80V) for 2-3 hours and stained with 1×SYBR gold for 15 minutes can provide an estimate of sample degradation, which would appear as a smear <10 kb. Finally, gDNA Screen Tape (Agilent) can be used to quickly assess DNA quality, though results can be variable. For reference, control gDNA used in this study without PreCR repair (as is standard for PacBio TPK2.0) had a DNA integrity number (DIN) of 9.7. In our hands, samples that were degraded and did not yield successful libraries had DIN <9.2. DNA can be purified using standard approaches such as phenol: chloroform: isoamyl alcohol extraction or commercially available products including Promega Wizard, New England BioLabs Monarch, and Qiagen MagAttract kits, which all produced gDNA with DIN >9.5 that could be successfully converted to SMRT-Tag libraries in our hands. Based on our experience, we suggest a minimum DIN of 9.5.

### Tagmentation Conditions

### Determining Conditions for an Application of Interest

The key parameter for Tn5-based PacBio library preparation is transposome concentration, which must be determined empirically for a given batch of Tn5 complexed with hairpin adaptors and for a given application. Note that input DNA mass and quality are also important considerations, but these may be constrained to a degree by the amount of material available, etc. In our hands, performing pilot experiments using a dilution series of transposome and/or input DNA obtained from a source comparable to the intended application are conducted for optimizing tagmentation. Analyzing libraries obtained from pilot studies via gel electrophoresis or on an instrument such as TapeStation, BioAnalyzer, or Femto Pulse (Agilent) is suggested. Multiplexing and sequencing libraries at low depth (e.g., FIGS. 9A-9C) can confirm that molecules in the expected length range are captured. The effect of transposome concentration, input DNA mass, and reaction temperature are discussed below.

### Transposome Concentration

Loading of Tn5 transposomes onto DNA can be approximated as a Poisson process (i.e., the number of Tn5 complexes per DNA fragment varies according to the amount of Tn5), and the exact position of each complex on single molecules is essentially random. The size of the resulting fragments, which represent the interstitial region between adjacent transposition sites, is thus the difference between adjacent realizations of a uniform random variable U(1, molecule length) and can be approximated by an exponential distribution. Therefore, under concentrations used for tagmentation, Tn5 has a tendence to generate short fragments.

The triple-mutant Tn5 enzyme used here permits transposome concentration-

dependent control of fragment lengths, which was confirmed initially based on analytical gel electrophoresis of tagmented gDNA (FIG. 1B). To better characterize the relationship between transposome concentration and fragment length, SMRT-Tag was performed on inputs ranging 40-1,000 ng and Tn5 monomer amounts of 0.005-5 pmol (at least two orders of magnitude for each parameter; FIGS. 9A-9C). Libraries were multiplexed and sequenced to low coverage, confirming the inverse relationship between Tn5 and DNA amounts on length. For example, 200 ng gDNA tagmented with the equivalent of 0.05 pmol Tn5 monomer at 55°° C. generated libraries of mean length ˜3-5 kb, whereas the same amount of DNA tagmented with 5 pmol Tn5 at 55° C. yielded molecules with ˜500 bp average length (FIGS. 9A-9C).

Given these observations, a simple procedure for calibrating the amount of hairpin-loaded Tn5 is proposed herein to generate a library of a specific mean size: First, using a fixed amount of gDNA (such as the 160 ng experiments in this study), carry out tagmentation with a dilution series (e.g., 1:16, 1:64, 1:128, etc.) of hairpin-loaded Tn5 stock (9.4 μM monomer) coupled with analytical electrophoresis or shallow multiplex sequencing to estimate the relationship between Tn5 quantity and library size distribution. Then, for a target library size (e.g., 3-5 kb), the amount of Tn5 can be normalized per mass gDNA (n pmol Tn5/m ng gDNA) to produce a ratio that is approximately scalable to a range of input quantities. As an example, for the transposomes assembled for this study, our experiments using 160 ng gDNA suggested that Tn5 monomer range from 0.073-0.146 pmol could consistently generate libraries with mean lengths of 2-5 kb. This yielded a Tn5 monomer: gDNA ratio of 4.6×10⁻⁴-9.3×10⁻⁴ (pmol:ng). Scaled to 40 ng gDNA, this gave a Tn5 amount of 0.018-0.037 pmol, which generated the expected library distributions of 2-5 kb (FIG. 9B).

This relationship was roughly observed to hold across the batches of barcoded hairpin-loaded Tn5 that were prepared in this study. Further, based on the particulars of the input material and assay, pilot experiments titrating different reaction conditions are the best way to guide parameter selection. For example, the amount of transposome required for in situ SAMOSA-Tag (wherein the transposition reaction occurs in intact nuclei) was much higher and determined based on reported concentrations used for ATAC-seq.

### Input DNA Mass

Tn5 tagmentation has a wide theoretical input range with lower bound on the picogram scale (i.e., single cells). Taking into consideration the mass/molar quantity tradeoff and minimum OPLC of 20-40 pM for PacBio sequencing noted above, the lowest amount of gDNA attempted to make libraries from in this study was 40 ng. In experiments that were performed to guide parameter selection (FIGS. 9A-9C), up to 1,000 ng of DNA was tagmented.

Though future modification of the protocol may enable use of large input amounts, it is considered that ˜250 ng to be a soft upper limit for tagmentation-based PacBio library preparation. Input DNA quality (see above) is an additional consideration that may affect the mass required for conversion to library molecules—i.e., for a low-quality sample, more input material would be required to generate sufficient sequenceable templates after exonuclease digestion.

### Reaction Temperature

Most library preparation protocols use Tn5 at 55° C., the temperature optimal for enzyme activity. However, Tn5 retains activity at lower temperatures. Both the conventionally used double-mutant and/or the triple-mutant enzymes used here have been shown in this study (FIGS. 1B, 9A-9C) and others⁵⁴ to favor generation of longer fragments at 37° C. Note that in contrast to the gel-based analysis of tagmented DNA in FIG. 1B, libraries generated under a variety of reaction conditions were multiplexed for sequencing in the analysis presented in FIGS. 9A-9C. Wide variation in length between libraries affected estimation of loading and sequencing characteristics, which may have obscured some temperature-dependent differences. Here, carrying out tagmentation at 55° C. was sufficient for generating libraries of mean lengths in the 1-7 kb range; however, in applications targeting much longer fragment lengths, it may be reasonable to lower the reaction temperature to 37° C. For example, in the context of SAMOSA-Tag, several ATAC-seq protocols use a lower temperature for tagmentation (37° C.) to better preserve native chromatin structure.

### Other Considerations

In this study, the effect of crowding agents (e.g., polyethylene glycol) on tagmentation efficiency and library characteristics was not directly tested. However, prior work suggests that modulating the type and concentration of crowding agents may help tune input quantity and library size⁵⁵.

### Size Selection

Bead-based cleanup can be optionally performed to shift the distribution of fragment sizes in the library at the cost of losing a portion of molecules. It is important to note that SMRT-Tag and SAMOSA-Tag libraries can generally be sequenced without size selection using polymerase 2.1/3.1 (see below). Given that Tn5 tagmentation is a Poisson process as described above, there can be a preponderance of short (<700 bp) fragments. These may be overlooked in fluorescence-based quantification assays despite constituting a significant fraction of the library. In cases where high concentrations of Tn5 are used or where preliminary quality control analyses suggest a large population of short fragments, depleting these molecules can improve loading efficiency by aligning the length distribution to the preference of polymerases 2.1/3.1 vs 2.2/3.2. Herein, depleting <700 bp or <3 kb fragments reduced the fraction of short reads in libraries sequenced with polymerase 2.2 and permitted more accurate estimation of mean fragment length during the sequencing loading reaction. The ‘double-sided’ cleanup wherein short and long fragments are sequenced separately is adapted from an older version of PacBio's Iso-Seq protocol in which short fragments depleted from the library are recovered and sequenced to maximize use of input DNA. This is not required for SMRT-Tag or SAMOSA-Tag but may be a consideration if starting material is limiting.

### Choice of PacBio Polymerase

Manufacturer recommendations suggest that libraries with mean fragment length <3kb should be sequenced with polymerase 2.1/3.1, whereas polymerases 2.2/3.2 are better suited for libraries with mean fragment length >3kb. This is based in part on general characteristics of the enzymes/sequencing chemistry—i.e., 2.2/3.2 polymerase is highly processive and produces longer reads but is generally less tolerant to poor estimation of mean library size during the loading process. In general, was found that libraries with mean lengths as high as ˜6 kb can be adequately sequenced with polymerase 2.1.

In Situ vs. Ex Situ SAMOSA-Tag

Both in situ (tagmentation occurs following EcoGII methylation in intact nuclei) and ex situ (DNA is purified from EcoGII methylated nuclei and then subjected to tagmentation) versions of the SAMOSA-Tag approach. Ex situ SAMOSA-Tag is essentially SMRT-Tag carried out using SAMOSA DNA as input, highlighting the flexibility of Tn5-based library preparation. Depending on the anticipated application, one approach may be preferred over the other. In situ tagmentation has the benefit of avoiding DNA extraction and attendant losses and preferentially samples open chromatin regions evinced by transposition adjacent to barrier elements (FIG. 3C) and ATAC-seq-like coverage profile (FIG. 24). This could be ideal in input and sequencing depth-limited settings where the primary biological interest is gene regulatory regions. On the other hand, ex situ SAMOSA-Tag delivers more uniform coverage as suggested by abrogation of the barrier effect (FIG. 3C) and may be better suited for applications requiring even genome sampling such as analysis of heterochromatic regions and integrated whole genome assembly and epigenome profiling.

### REFERENCES

1. Logsdon, G. A., Vollger, M. R. & Eichler, E. E. Long-read human genome sequencing and its applications. Nat. Rev. Genet. 21, 597-614 (2020).
2. Aganezov, S. et al. A complete reference genome improves analysis of human genetic variation. Science 376, eab13533 (2022).
3. Vollger, M. R. et al. Segmental duplications and their variation in a complete human genome. Science 376, eabj6965 (2022).
4. Abdulhay, N. J. et al. Massively multiplex single-molecule oligonucleosome footprinting. Elife 9, (2020).
5. Stergachis, A. B., Debo, B. M., Haugen, E., Churchman, L. S. & Stamatoyannopoulos, J. A. Single-molecule regulatory architectures captured by chromatin fiber sequencing. Science 368, 1449-1454 (2020).
6. Shipony, Z. et al. Long-range single-molecule mapping of chromatin accessibility in eukaryotes. Nat. Methods 17, 319-327 (2020).
7. Lee, I. et al. Simultaneous profiling of chromatin accessibility and methylation on human cell lines with nanopore sequencing. Nat. Methods 17, 1191-1199 (2020).
8. Altemose, N. et al. DiMeLo-seq: a long-read, single-molecule method for mapping protein-DNA interactions genome wide. Nat. Methods 19, 711-723 (2022).
9. Au, K. F. et al. Characterization of the human ESC transcriptome by hybrid sequencing. Proc. Natl. Acad. Sci. U. S. A. 110, E4821-30 (2013).
10. Sharon, D., Tilgner, H., Grubert, F. & Snyder, M. A single-molecule long-read survey of the human transcriptome. Nat. Biotechnol. 31, 1009-1014 (2013).
11. Abdulhay, N. J. et al. Nucleosome density shapes kilobase-scale regulation by a mammalian chromatin remodeler. Nat. Struct. Mol. Biol. (2023) doi: 10.1038/s41594-023-01093-6.

12. Wang, Y. et al. Single-molecule long-read sequencing reveals the chromatin basis of gene expression. Genome Res. 29, 1329-1342 (2019).

13. Quail, M. A. et al. A tale of three next generation sequencing platforms: comparison of Ion Torrent, Pacific Biosciences and Illumina MiSeq sequencers. BMC Genomics 13, 341 (2012).
14. Adey, A. et al. Rapid, low-input, low-bias construction of shotgun fragment libraries by high-density in vitro transposition. Genome Biol. 11, R119 (2010).
15. Adey, A. & Shendure, J. Ultra-low-input, tagmentation-based whole-genome bisulfite se1quencing. Genome Res. 22, 1139-1143 (2012).
16. Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat. Methods 10, 1213-1218 (2013).
17. Schmidl, C., Rendeiro, A. F., Sheffield, N. C. & Bock, C. ChIPmentation: fast, robust, low-input ChIP-seq for histones and transcription factors. Nat. Methods 12, 963-965 (2015).
18. Chen, C. et al. Single-cell whole-genome analyses by Linear Amplification via Transposon Insertion (LIANTI). Science 356, 189-194 (2017).
19. Minussi, D. C. et al. Breast tumours maintain a reservoir of subclonal diversity during expansion. Nature 592, 302-308 (2021).
20. Payne, A. C. et al. In situ genome sequencing resolves DNA sequence and structure in intact biological samples. Science 371, eaay3446 (2021).
21. Cusanovich, D. A. et al. Epigenetics. Multiplex single-cell profiling of chromatin accessibility by combinatorial cellular indexing. Science 348, 910-914 (2015).
22. Cao, J. et al. Joint profiling of chromatin accessibility and gene expression in thousands of single cells. Science 361, 1380-1385 (2018).
23. Yin, Y. et al. High-throughput single-cell sequencing with linear amplification. Mol. Cell 76, 676-690.e10 (2019).

124. Eid, J. et al. Real-time DNA sequencing from single polymerase molecules. Science 323, 133-138 (2009).
25. Hennig, B. P. et al. Large-s low-cost NGS library preparation using a robust Tn5 purification and tagmentation protocol. G3: Genes, Genomes, Genetics 8, 79-89 (2018).
26. Reznikoff, W. S. Tn5 as a model for understanding DNA transposition. Mol. Microbiol. 47, 1199-1206 (2003).
27. Zook, J. M. et al. Extensive sequencing of seven human genomes to characterize benchmark reference materials. Scientific data vol. 3 160025 (2016).
28. Krusche, P. et al. Best practices for benchmarking germline small-variant calls in human genomes. Nat. Biotechnol. 37, 555-560 (2019).
29. Flusberg, B. A. et al. Direct detection of DNA methylation during single-molecule, real-time sequencing. Nat. Methods 7, 461-465 (2010).
30. Simpson, J. T. et al. Detecting DNA cytosine methylation using nanopore sequencing. Nat. Methods 14, 407-410 (2017).
31. Grandi, F. C., Modi, H., Kampman, L. & Corces, M. R. Chromatin accessibility profiling by ATAC-seq. Nat. Protoc. 17, 1518-1552 (2022).
32. Sayles, L. C. et al. Genome-Informed Targeted Therapy for Osteosarcoma. Cancer Discov. 9, 46-63 (2019).
33. Vitak, S. A. et al. Sequencing thousands of single-cell genomes with combinatorial indexing. Nat. Methods 14, 302-308 (2017).
34. Ibarra, A., Benner, C., Tyagi, S., Cool, J. & Hetzer, M. W. Nucleoporin-mediated regulation of cell identity genes. Genes Dev. 30, 2253-2258 (2016).
35. Traag, V. A., Waltman, L. & van Eck, N. J. From Louvain to Leiden: guaranteeing well-connected communities. Sci. Rep. 9, 5233 (2019).
36. Wang, H. et al. Widespread plasticity in CTCF occupancy linked to DNA methylation. Genome Res. 22, 1680-1688 (2012).
37. Abdulhay, N. J. et al. Single-fiber nucleosome density shapes the regulatory output of a mammalian chromatin remodeling enzyme. bioRxiv 2021.12.10.472156 (2021) doi: 10.1101/2021.12.10.472156.
38. Nguyen, H. G. et al. Development of a stress response therapy targeting aggressive prostate cancer. Sci. Transl. Med. 10, (2018).
39. Alpsoy, A. et al. BRD9 Is a Critical Regulator of Androgen Receptor Signaling and Prostate Cancer Progression. Cancer Res. 81, 820-833 (2021).
40. Shan, Z. et al. CTCF regulates the FoxO signaling pathway to affect the progression of prostate cancer. J. Cell. Mol. Med. 23, 3130-3139 (2019).
41. Wang, T. et al. Integrative epigenome map of the normal human prostate provides insights into prostate cancer predisposition. Front. Cell Dev. Biol. 9, 723676 (2021).
42. Xiao, L. et al. Targeting SWI/SNF ATPases in enhancer-addicted prostate cancer. Nature 601, 434-439 (2022).
43. Ramani, V. et al. Massively multiplex single-cell Hi-C. Nat. Methods 14, 263-266 (2017).
44. Liu, M. H. et al. Single-strand mismatch and damage patterns revealed by single-molecule DNA sequencing. bioRxiv (2023) doi: 10.1101/2023.02.19.526140.

45. Bruinsma, S. et al. Bead-linked transposomes enable a normalization-free workflow for NGS library preparation. BMC Genomics 19, 722 (2018).
46. Meers, M. P., Bryson, T. D., Henikoff, J. G. & Henikoff, S. Improved CUT&RUN chromatin profiling tools. Elife 8, (2019).
47. Gilpatrick, T. et al. Targeted nanopore sequencing with Cas9-guided adapter ligation. Nat. Biotechnol. 38, 433-438 (2020).
48. Emiliani, F. E., Hsu, I. & McKenna, A. Circuit-seq: Circular reconstruction of cut in vitro transposed plasmids using Nanopore sequencing. bioRxiv (2022) doi: 10.1101/2022.01.25.477550.
49. Al'Khafaji, A. M. et al. High-throughput RNA isoform sequencing using programmable cDNA concatenation. bioRxiv 2021.10.01.462818 (2021) doi: 10.1101/2021.10.01.462818.
50. English, A. C., Menon, V. K., Gibbs, R. A., Metcalf, G. A. & Sedlazeck, F. J. Truvari: refined structural variant comparison preserves allelic diversity. Genome Biol. 23, 271 (2022).
51. ENCODE Project Consortium. An integrated encyclopedia of DNA elements in the human genome. Nature 489, 57-74 (2012).
52. Storey, J. D. & Tibshirani, R. Statistical significance for genomewide studies. Proc. Natl. Acad. Sci. U. S. A. 100, 9440-9445 (2003).
53. Yu, H.-B., Johnson, R., Kunarso, G. & Stanton, L. W. Coassembly of REST and its cofactors at sites of gene repression in embryonic stem cells. Genome Res. 21, 1284-1293 (2011).
54. Vonesch, S. C. et al. Fast and inexpensive whole-genome sequencing library preparation from intact yeast cells. G3 (Bethesda) 11, 1-12 (2021).
55. Picelli, S. et al. Tn5 transposase and tagmentation procedures for massively scaled sequencing projects. Genome Res. 24, 2033-2040 (2014).

### OTHER EMBODIMENTS

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed:

1. A method of genome and epigenome sequencing, comprising:
isolating DNA sequences, obtaining one or more cells or nuclei from a sample;
conducting a tagmentation reaction with a hyperactive transposase on the isolated DNA sequences cells or nuclei to produce a plurality of nucleic acid libraries;
repairing gaps in nucleic libraries;
fractionating the nucleic acid libraries; and,
sequencing the nucleic acid libraries.

2. The method of claim 1, wherein the isolated DNA sequence concentration is in a range from about 10 ng to about 100 ng.

3. (canceled)

4. (canceled)

5. (canceled)

6. The method of claim 1, wherein the isolated DNA sequence concentration about 35 ng to about 60 ng.

7. The method of claim 1, wherein the isolated DNA sequence concentration is about 40 ng.

8. The method of claim 1, wherein a plurality of cells or nuclei are subjected to the tagmentation reaction.

9. The method of claim 8, wherein a single cell or nucleus is subjected to the tagmentation reaction.

10. The method of claim 1, wherein the hyperactive transposase controls fragment size based on concentration of the isolated DNA sequences.

11. The method of claim 10, wherein the hyperactive transposase comprises hairpin oligonucleotides to generate long fragments.

12. The method of claim 1, wherein long fragments generated comprise up to about 150,000 base pairs.

13. The method of claim 12, wherein a generated fragment comprises about 100 base pairs to about 150,000.

14. The method of claim 1, wherein the hyperactive transposase is prokaryotic, eukaryotic or proteases.

15. The method of claim 1, wherein the prokaryotic hyperactive transposases comprise Tn5, Tn5 mutants, Tn5 derivatives, Tn7, Tn10, phages or combinations thereof.

16. The method of claim 15, wherein a Tn5 mutant comprises one or more mutations.

17. The method of claim 16, wherein the Tn5 mutant comprises an R27S, an E54K, an L372P substitution or combinations thereof.

18. The method of claim 15, wherein a Tn5 derivative is linked to an epitope comprising protein A, nanobodies, biotin, streptavidin, protein G, FK-binding protein, beads or combinations thereof.

19. The method of claim 15, wherein the protease transposases comprise casposases, Cas9 or combinations thereof, and the eukaryotic transposases comprise retrotransposons (class I transposons), class II transposons or miniature inverted-repeat transposable elements (MITEs, or class III transposons).

20. (canceled)

21. The method of claim 19, wherein the eukaryotic transposases comprise Sleeping Beauty transposon system (SBTS), piggyBac (PB) transposons, Hermes transposons or combinations thereof.

22. The method of claim 1, wherein the sequencing is a high-throughput sequencing reaction.

23. The method of claim 22, wherein the sequencing is a single molecule sequencing (SMS) method.

24. The method of claim 1, wherein a ratio of transposase: DNA is from about 1×10⁻⁵ to 1×10⁻³ picomoles of per ng of DNA.

25. The method of claim 19 , wherein a ratio of transposase: DNA is from about 5×10⁻⁴ to 10×10⁻³ picomoles of per ng of DNA.

26. The method of claim 1, wherein the tagmentation reaction is conducted at a temperature between 15° C. to about 75° C.

27. The method of claim 1, wherein the tagmentation reaction is conducted at a temperature of about 55° C.

28. The method of claim 1, wherein the libraries comprise one or more multiplexed nucleic acid sequences.

29. The method of claim 1, wherein each transposon further comprises a unique barcode.

30. The method of claim 1, wherein the sample is a biological sample.

31. The method of claim 1, wherein the method does not comprise the step of amplification of the libraries.

32. A nucleic acid sequencing assay comprising:
modifying one or more cells or cell nuclei in situ;
tagmenting the cells or cell nuclei with a hairpin-loaded hyperactive transposon;
extracting DNA from the cell nuclei;
conducting gap repair of the extracted DNA; and, sequencing of the DNA.

33. The method of claim 32, wherein the modification comprises methylation, acetylation, phosphorylation, ubiquitination, sumoylation or combinations thereof.

34. The method of claim 33, wherein the modification comprises methylation.

35. The method of claim 32, wherein the cells or cell nuclei are simultaneously subjected to nucleolytic cleavage and DNA modification.

36. The method of claim 32, wherein the cells or cell nuclei are subjected to nucleolytic cleavage after DNA modification.

37. The method of claim 36, wherein the nucleolytic cleavage is conducted by a nuclease.

38. The method of claim 37, wherein the nuclease is a micrococcal nuclease (MNase).

39. The method of claim 32, wherein the one or more cells or cell nuclei comprise from about 500 cells or cell nuclei to about 200,000 cells or cell nuclei.

40. (canceled)

41. The method of claim 32, wherein the one or more cells or cell nuclei comprises from about 1000 cells or cell nuclei to about 100,000 cells or cell nuclei.

42. The method of claim 32, wherein the one or more cells or cell nuclei comprise a single nucleus.

43. The method of claim 32, wherein the hyperactive transposase controls fragment size based on concentration of the isolated DNA sequences.

44. The method of claim 32, wherein the hyperactive transposase comprises hairpin oligonucleotides to generate long fragments.

45. (canceled)

46. The method of claim 44, wherein a generated fragment comprises about 100 base pairs to about 150,000.

47. The method of claim 32, wherein the hyperactive transposase is prokaryotic, eukaryotic or proteases.

48. The method of claim 47, wherein the prokaryotic hyperactive transposases comprise Tn5, Tn5 mutants, Tn5 derivatives, Tn7, Tn10, phages or combinations thereof.

49. The method of claim 48, wherein a Tn5 mutant comprises one or more mutations, comprising an R27S, an E54K, an L372P substitution or combinations thereof.

50. (canceled)

51. The method of claim 48, wherein a Tn5 derivative is linked to an epitope comprising protein A, nanobodies, biotin, streptavidin, protein G, FK-binding protein, beads or combinations thereof.

52. The method of claim 48, wherein the protease transposases comprise casposases, Cas9 or combinations thereof.

53. The method of claim 48, wherein the eukaryotic transposases comprise retrotransposons (class I transposons), class II transposons or miniature inverted-repeat transposable elements (MITEs, or class III transposons).

54. The method of claim 53, wherein the eukaryotic transposases comprise Sleeping Beauty transposon system (SBTS), piggyBac (PB) transposons, Hermes transposons or combinations thereof.

55. The method of claim 32, wherein the sequencing is a high-throughput sequencing reaction or a single molecule sequencing (SMS) method.

56. (canceled)

57. The method of any one of claims 52-56, wherein the ratio of transposase: DNA is from about 1×10⁻⁵ to 1×10⁻³ picomoles of per ng of DNA.

58. The method of any one of claims 52-56, wherein the ratio of transposase: DNA is from about 5×10⁻⁴ to 1×10⁻³ picomoles of per ng of DNA.

59. The method of claim 32, wherein the tagmentation reaction is conducted at a temperature between 15° C. to about 75° C.

60. The method of claim 32, wherein the tagmentation reaction is conducted at a temperature of about 55° C.

61. The method of claim 32, wherein the libraries comprise one or more multiplexed nucleic acid sequences.

62. The method of claim 32, wherein each transposon further comprises a unique barcode.

63. The method of claim 32, wherein the sample is a biological sample.

64. The method of any one of claims 32, wherein the method does not comprise the step of amplification of the libraries.

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)
